SEQUENCE LISTING (12 sequences)

SEQ ID NO: 1 - DNA, 21 bp, Homo sapiens
aggcgtgaagagcaagggaaa

SEQ ID NO: 2 - DNA, 22 bp, Homo sapiens
ccccatcttcactcagagactg

SEQ ID NO: 3 - DNA, 19 bp, Homo sapiens
ctacgggacagcgtgcagc

SEQ ID NO: 4 - DNA, 23 bp, Homo sapiens
cagcatgattggtcctcagttcc

SEQ ID NO: 5 - DNA, 22 bp, Homo sapiens
gctgacaaaggccaggaacagc

SEQ ID NO: 6 - DNA, 20 bp, Homo sapiens
acctcccccagcacctcagc

SEQ ID NO: 7 - DNA, 21 bp, Homo sapiens
gcaagcctgttggagactcag

SEQ ID NO: 8 - DNA, 19 bp, Homo sapiens
ctgtcccgtagcaccttct

SEQ ID NO: 9 - DNA, 20 bp, Homo sapiens
gacctggactgaagttcgca

SEQ ID NO: 10 - DNA, 21 bp, Homo sapiens
acttgcttgctaaatcaggga

SEQ ID NO: 11 - DNA, 20 bp, Homo sapiens
ggcctccaaggagtaagacc

SEQ ID NO: 12 - DNA, 20 bp, Homo sapiens
aggggtctacatggcaactg

(19) United States
(12) Patent Application Publication
(10) Pub. No.: US 2015/0275177 A1
(43) Pub. Date: Oct. 1, 2015

(54) Methods For Generating Pluripotent Stem Cell-Derived Brown Fat Cells

(71) Applicant: BioTime, Inc.,, Alameda, CA (US)

(72) Inventors: Michael West, Mill Valley, CA (US); Hal Sternberg, Berkeley, CA (US)

(21) Appl. No.: 14/554,019

(22) Filed: Nov. 25, 2014

Related U.S. Application Data
(60) Provisional application No. 61/908,621, filed on Nov. 25, 2013.
(60) Provisional application No. 62/020,343, filed on Jul. 2, 2014.

(51) Int. Cl. C12N 5/077

(52) U.S. Cl. CPC C12N 5/0653; C12N 2502/03; C12N 2501/155; C12N 2501/385

(57) ABSTRACT

Aspects of the present invention include methods and compositions related to the production and use of pluripotent stem cell-derived clonal embryonic progenitor cell types useful in the generation of cellular components of brown adipocyte tissue for research and therapy relating to applications in obesity, diabetes, and cardiovascular disease.

### CROSS-REFERENCE TO RELATED APPLICATIONS

This application claims priority to U.S. Provisional Application No. 61/908,621, filed on Nov. 25, 2013 and U.S. Provisional Application No. 62/020,343, filed on Jul. 2, 2014, the entire contents of both of which are hereby incorporated by reference.

### FIELD OF THE INVENTION

The invention relates to the field of stem cell biology.

### BACKGROUND OF THE INVENTION

Advances in stem cell technology, such as the isolation and propagation in vitro of primordial stem cells, including embryonic stem cells (“ES” cells including human ES cells (“hES” cells)) and related primordial stem cells including but not limited to, iPS, EG, EC, ICM, epiblast, or ED cells (including said cells from the human species), constitute an important new area of medical research and therapeutic product development. Many of these primordial stem cells are naturally telomerase positive in the undifferentiated state, thereby allowing the cells to be expanded extensively and subsequently genetically modified and clonally expanded after said genetic modification prior to differentiation. Telomere length in many of the primordial cells lines is comparable to that observed in sperm DNA (approximately 10-18 kb TRF length) through in part the expression of the catalytic component of telomerase (TERT). Therefore, while differentiated progeny of the primordial stem cells are typically mortal due to the repression of TERT expression and telomere length shortens with cell doubling, their long initial telomere lengths provide the cells with a long replicative capacity compared to fetal or adult-derived cells.

Human ES cells have a demonstrated potential to be propagated in the undifferentiated state and then to be subsequently induced to differentiate into any and all of the cell types in the human body, including complex tissues. The pluripotency of hES cells has led to the suggestion that many diseases resulting from dysfunction of cells may be amenable to treatment by the administration of hES-derived cells of various differentiated types (Thomson et al., Science 282:1145-1147 (1998)), and the long proliferative lifespan of hES-derived progenitor lines has allowed the clonal expansion and initial characterization of hES cell-derived embryonic progenitor cell lines (West et al, Regen Med (2008) 3(3), 287-308).

Nuclear transfer studies have demonstrated that it is possible to transform a somatic differentiated cell back to a primordial stem cell state such as that of embryonic stem (“ES”) cells (Cibelli et al., Nature Biotech 16:642-646 (1998)) or embryo-derived (“ED”) cells. Technologies to reprogram somatic cells back to a totipotent ES cell state, such as by the transfer of the genome of the somatic cell to an enucleated oocyte and the subsequent culture of the reconstructed embryo to yield ES cells, often referred to as somatic cell nuclear transfer (“SCNT”) or through analytical reprogramming technology wherein somatic cells are reprogrammed using transcriptional regulators (see PCT application Ser. No. PCT/US2006/030632 filed on Aug. 3, 2006 and titled “Improved Methods of Reprogramming Animal Somatic Cells”) have been described. These methods offer potential strategies to transplant primordial-derived somatic cells with a nuclear genotype of the patient (Lanza et al., Nature Medicine 5:975-977 (1999)).

In addition to SCNT and analytical reprogramming technologies, other techniques exist to address the problem of transplant rejection, including the use of gynogenesis and androgenesis (see U.S. application No. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US00/29551, filed Oct. 27, 2000). In the case of a type of gynogenesis designated parthenogenesis, pluripotent stem cells may be manufactured without antigens foreign to the gamete donor and therefore useful in manufacturing cells that can be transplanted without rejection into the gamete donor. In addition, parthenogenic stem cell lines can be assembled into a bank of cell lines homozygous in the HLA region (or corresponding MHC region of nonhuman animals) to reduce the complexity of a stem cell bank in regard to HLA haplotypes.

Cell lines or a bank of said cell lines can be produced that are hemizygous in the region of the chromatin containing the HLA genes (or corresponding MHC region of nonhuman animals; see PCT application Ser. No. PCT/US2006/040985 filed Oct. 20, 2006 entitled “Totipotent, Nearly Totipotent or Pluripotent Mammalian Cells Homozygous or Hemizygous for One or More Histocompatibility Antigen Genes”). A bank of hemizygous cell lines provides the advantage of not only reducing the complexity inherent in the normal mammalian MHC gene pool, but it also reduces the gene dosage of the antigens to reduce the expression of said antigens without eliminating their expression entirely, thus avoiding stimulation of a natural killer response.

In addition to reprogramming by SCNT or analytical reprogramming technologies such as iPS cell generation, the pluripotent stem cells may be genetically modified to reduce immunogenicity through the modulation of expression of certain genes such as the knockout of HLA genes, one of both alleles of beta 2 microglobulin (B2M), increased expression of HLA-G or HLA-H, or CTLA4-Ig and PD-L1 (Z. Rong et al, An Effective Approach to Prevent Immune Rejection of Human ESC-Derived Allografts, Cell Stem Cell, 14: 121-130 (2014) incorporated herein by reference, as well as other modifications known in the art and subsequently used to generate differentiated cells for research and therapeutic applications. Such genetically-modified promoridial stem cells designed to produce cells with reduced immunogenicity are designated “universal donor cells” herein.

In regard to differentiating primordial stem cells into desired cell types, the potential to isolate human pluripotent stem cell-derived clonal embryonic progenitor cell lines provides a means to propagate novel highly purified cell lineages with a prenatal pattern of gene expression useful for regenerating tissues. Such cell types have important applications in research, and for the manufacture of cell-based therapies (see PCT application Ser. No. PCT/US2006/013519 filed on Apr. 11, 2006 and entitled “Novel Uses of Cells With Prenatal Patterns of Gene Expression”; U.S. patent application Ser. No. 11/604,047 filed on Nov. 21, 2006 and entitled “Methods to Accelerate the Isolation of Novel Cell Strains from Pluripotent Stem Cells and Cells Obtained Thereby”; and U.S. patent application Ser. No. 12/504,630 filed on Jul. 16, 2009 and entitled “Methods to Accelerate the Isolation of Novel Cell Strains from Pluripotent Stem Cells and Cells Obtained Thereby”); U.S. patent application Ser. No. 14/048,910 entitled “Differentiated Progeny of Clonal Progenitor Cell Lines,” incorporated herein by reference. Clonal, oligoclonal, and pooled populations of clonal and oligoclonal embryonic progenitors capable of forming embryonic cutaneous adipocyte progenitor cells (ECAPCs) expressing EYA4, wherein said progenitor cells are capable of differentiating into certain cellular components of brown adipose tissue (BAT) have also been disclosed (see WO2011/150105 entitled “Improved Methods of Screening Embryonic Progenitor Cell Lines,”) as well as (U.S. patent application Ser. No. 13/683,241, entitled “Methods of Screening Embryonic Progenitor Cell Lines”).

Despite of the advances described above, there remains a need to improve methods for screening pluripotent stem cell-derived cells for potential of differentiation into desired cell types, including the cellular components of BAT. There also remains a need for means to effectively differentiate pluripotent stem cells into site-specific progenitor and terminally differentiated cell types. Moreover, there is a growing need for improved methods for generating progenitor cell types from pluripotent stem cells that display and maintain a uniform differentiated state and exhibit site-specific differences in gene expression. Adipocytes are an example of a cell type with important site-specific differences in gene expression, with diverse types of adipocytes within the human body each having unique roles in maintaining physiological homeostasis. While adipocytes in general provide a physiological function of storing energy for future metabolic needs, a specialized type of adipose tissue called brown adipose tissue (BAT) regulates energy expenditure or thermogenesis. BAT cells are progressively lost during the development and aging of humans, consequently increasing the risk of disorders where BAT cells play a critical role (such as in regulating fat metabolism in the body, blood pressure, blood glucose regulation, pancreatic beta cell numbers in the pancreas, and HDL and LDL lipoprotein and triglyceride metabolism) in the populations with less BAT. Thus, a need exists for generating purified adipocyte progenitors capable of differentiating into site-specific adipocytes of diverse tissue types, including BAT cells.

Surprisingly, the methods of the present invention demonstrate that distinct pluripotent stem cell-derived clonal embryonic progenitor cell lines can be isolated which when cultured and expanded in the undifferentiated state do not express high levels of adipocyte markers and do not express detectable levels of markers of BAT adipocytes such as the gene UCP1 or the adipokine ADIPOQ, but nevertheless, under certain conditions disclosed herein, are capable of differentiating into either: 1) UCP1-expressing brown adipose tissue (BAT) cells that express low to undetectable adipokines such as C19orf80 (also known betatrophin or ANGPTL8, encoded in humans by the C19orf80 gene), and adiponectin (also known as AdipoQ or GBP-28, encoded in humans by the ADIPOQ gene) or 2) clonal embryonic progenitors capable of making adipocytes that express abundant mRNA for C19orf80 and adiponectin, but low levels of UCP1. In addition, surprisingly, the methods of the present invention demonstrate that the pluripotent stem cell-derived clonal embryonic progenitor cell line ESI EP004 NP 110SM (also referred to as NP 110SM) which can be cultured and expanded in a relatively undifferentiated state that does not express pluripotency markers or express high levels of adipocyte markers and does not express detectable levels of markers of BAT adipocytes such as C19orf80, adiponectin or UCP1, nevertheless, when differentiated using the methods of the present invention, is capable of simultaneously expressing levels of UCP1, C19orf80, and ADIPOQ at levels comparable or higher to cultured fetal-tissue derived BAT cells.

There is a need for methods that permit the directed differentiation of pluripotent stem cells into particular progenitor cell types capable of making the cellular components of brown fat that can be effectively and reproducibly dosed in cell therapy regimens that result in the engraftment of viable and functional BAT cells useful in the treatment of the symptoms of adiposity, Type I and Type II diabetes, hypertension, and diseases associated with endothelial cell dysfunction including coronary disease syndromes where many of these disorders occur simultaneously in a patient (such as metabolic syndrome X and related disorders as described herein). Moreover, there is a need for progenitor cell types and terminally differentiated cell types with expression of physiologically-beneficial genes including, but not limited to, UCP1, C19orf80 and ADIPOQ, and formulating said cells such that they may be stably engrafted subcutaneously and may deliver such adipokines and beneficial factors systemically to increase insulin sensitivity, decrease total body fat, decrease symptoms of Type I and Type II diabetes, favorably impact the course of coronary disease, and treat metabolic syndrome X. Lastly, there exists a need for a biocompatible matrix that facilitates the differentiation of embryonic progenitors into adipocytes, to promote the permanentengraftment of said cells in suitable sites in the body, and limit the undesired migration of said brown fat cellular components sites when injected in vivo.

Various embodiments of the invention described infra meet these needs and other needs in the field.

### SUMMARY

The present invention provides compounds, compositions, kits, reagents and methods useful for the differentiation and use of human embryonic progenitor cell types.

In one embodiment, the invention provides methods of generating novel pluripotent stem cell-derived cellular components of brown adipose tissue, compositions comprising the same, and methods of using the same.

In further embodiments the invention provides isolated clonal progenitor cell lines that give rise to diverse types of brown adipose cells. The isolated clonal progenitor cell lines may give rise to brown adipose cells in vitro. The isolated clonal progenitor cell lines may give rise to brown adipose cells in vivo.

In certain embodiments the invention provides an isolated pluripotent stem cell-derived clonal progenitor cell line capable of differentiating into a cellular component of BAT, wherein said differentiated cell, derived from a relatively undifferentiated progenitor cell, expresses one or more markers chosen from FABP4, C19orf80, ADIPQ, UCP1, PCK1, NNAT, THRSP, CEBPA, or CIDEA after being differentiated as described herein, but unlike fetal or adult-derived BAT cells, said pluripotent stem cell-derived clonal progenitor cell line does not express the gene COX7A1 when cultured and differentiated in vitro prior to in vivo administration. The isolated clonal progenitor cell line may give rise to brown adipose cells in vitro. The isolated clonal progenitor cell line may give rise to brown adipose cells in vivo.

In other embodiments the invention provides an isolated clonal progenitor cell line differentiated such that it expresses one or more markers chosen from FABP4, C19orf80, ADIPOQ, and low to undetectable levels of UCP1. The isolated clonal progenitor cell line may give rise to one type of brown adipose cells in vitro. The isolated clonal progenitor cell line may also give rise to brown adipose cells in vivo when formulated and transplanted as described herein.

In yet other embodiments the invention provides an isolated clonal progenitor cell line capable of differentiating into adipocytes that express FABP4, and UCP1, but do not express or express at low levels C19orf80 or ADIPOQ. The isolated clonal progenitor cell line may give rise to brown adipose cells in vitro distinct from those expressing ADIPOQ and C19orf80. The isolated clonal progenitor cell line may give rise to brown adipose cells in vivo when formulated and transplanted as described herein. The clonal progenitor cell lines capable of differentiating into adipocytes that express the markers FABP4, C19orf80, and ADIPOQ, and low to undetectable levels of UCP1 may be formulated in a mixture with clonal progenitor cell lines capable of differentiating into adipocytes that express FABP4, and UCP1, but do not express C19orf80, or ADIPOQ to restore healthy levels of adipokines and to generate weight loss in patients afflicted with obesity, hypertension, Type I or Type II diabetes, and coronary disease.

In other embodiments the invention provides isolated clonal progenitor cell lines expressing C19orf80. The isolated clonal progenitor cell line may give rise to brown adipose cells in vitro. The isolated clonal progenitor cell line may give rise to brown adipose cells in vivo.

In further embodiments the invention provides an isolated clonal progenitor cell line expressing UCP1. The isolated clonal progenitor cell line may give rise to brown adipose cells in vitro. The isolated clonal progenitor cell line may give rise to brown adipose cells in vivo.

In still other embodiments the invention provides a combined formulation of isolated clonal progenitor cell lines expressing C19orf80 and UCP1. The combination of isolated clonal progenitor cell lines may give rise to brown adipose cells in vitro. The isolated clonal progenitor cell lines may also give rise to brown adipose cells in vivo.

In other embodiments, the invention provides methods of maximizing the expression of desired genes in said brown fat cells, compositions regarding the same and methods of using the same.

In other embodiments the invention provides a method of obtaining embryonic cellular progenitors of BAT wherein the clonal progenitor cells when cultured in a relatively undifferentiated progenitor state synchronized in quiescence as described herein express one or more gene expression markers chosen from DLK1, HOXA5, SLC7A14, NTNG1, HEPH, PGM5, IL13RA2, SLC1A3, and SBSN but unlike fetal or adult-derived BAT progenitors do not express COX7A1, the method comprising contacting the clonal progenitor cell line with one or more TGFβ family members, optionally also contacting the clonal progenitor cell line with a PPARγ agonist, thereby obtaining a cell expressing one or more markers chosen from FABP4, C19orf80, ADIPQ, UCP1, PCK1, NNAT, THRSP, CEBPA, CIDEA, but not expressing COX7A1. Suitable TGFβ family members include members of the BMP family, such as BMP4, BMP6. In some embodiments the TGFβ family member may be TGFβ3. The clonal progenitor cell line may be grown on or encased in a hydrogel, e.g. a hydrogel comprising thiolated hyaluronate, thiolated gelatin and/or both thiolated hyaluronate and thiolated gelatin.

In still other embodiments the invention provides a method of obtaining embryonic cellular progenitors of BAT wherein the clonal progenitor cells when cultured in a relatively undifferentiated progenitor cell synchronized into quiescence as described herein express one or more of gene expression markers chosen from POSTN, KRT34, MKX, HAND2, TBX15, HOXA10, PLXDC2, DHRS9, NNAT, and HOXD11, but do not express COX7A1, DLK1, EYA4, SLC7A14, or NTNG1, the method comprising contacting the clonal progenitor cell line with one or more TGFβ family members, optionally also contacting the clonal progenitor cell line with a PPARγ agonist, thereby obtaining a cell expressing one or more markers chosen from FABP4, C19orf80, ADIPQ, UCP1, PCK1, NNAT, THRSP, CEBPA, CIDEA, but not expressing COX7A1. Suitable TGFβ family members include members of the BMP family, such as BMP4, BMP6. A nonlimiting example of a suitable PPARγ agonist is rosiglitazone. In some embodiments the TGFβ family member may be TGFβ3. The clonal progenitor cell line may be grown on or encased in a hydrogel, e.g. a hydrogel comprising thiolated hyaluronate, thiolated gelatin and/or both thiolated hyaluronate and thiolated gelatin.

In still other embodiments the invention provides a method of obtaining embryonic cellular progenitors of BAT wherein the clonal progenitor cells when cultured in a relatively undifferentiated progenitor cell synchronized into quiescence as described herein express one or more of gene expression markers chosen from TAC1, SCARA5, EYA4, or TBX1), but do not express HOXA10 or IL13RA2, the method comprising contacting the clonal progenitor cell line with one or more TGFβ family members, optionally also contacting the clonal progenitor cell line with a PPARγ agonist, thereby obtaining a cell expressing one or more markers chosen from FABP4, C19orf80, ADIPQ, UCP1, PCK1, NNAT, THRSP, CEBPA, CIDEA, but not expressing COX7A1. Suitable TGFβ family members include members of the BMP family, such as BMP4, BMP6. A nonlimiting example of a suitable PPARγ agonist is rosiglitazone. In some embodiments the TGFβ family member may be TGFβ3. The clonal progenitor cell line may be grown on or encased in a hydrogel, e.g. a hydrogel comprising thiolated hyaluronate, thiolated gelatin and/or both thiolated hyaluronate and thiolated gelatin.

In yet other embodiments the invention provides a method of obtaining a cell expressing a plurality of markers chosen from FABP4, C19ORF80, ADIPQ, UCP1, PCK1, NNAT, THRSP, CEBPA, and CIDEA, the method comprising contacting one of the three progenitor cell types described above (wherein the cells when cultured in a relatively undifferentiated progenitor state synchronized in quiescence as described herein express gene expression markers chosen from: 1) DLK1, HOXA5, SLC7A14, NTNG1, HEPH, PGM5, IL13RA2, SLC1A3, and SBSN but unlike fetal or adult-derived BAT progenitors do not express COX7A1, or 2) express one or more markers chosen from POSTN, KRT34, MKX, HAND2, TBX15, HOXA10, NNAT, and HOXD11, but do not express COX7A1, DLK1, EYA4, SLC7A14, or NTNG1, or 3) express one or more markers chosen from TAC1, SCARA5, EYA4, or TBX1), but do not express HOXA10 or IL13RA2) with one or more TGFβ family members, optionally also contacting the clonal progenitor cell line with a PPARγ agonist, thereby obtaining a cell expressing one or more markers chosen from FABP4, C19orf80, ADIPQ, UCP1, PCK1, NNAT, THRSP, CEBPA, CIDEA, but still not expressing COX7A1. Suitable TGFβ family members include members of the BMP family, such as BMP4, BMP6. A nonlimiting example of a PPARγ agonist is rosiglitazone. In some embodiments the TGFβ family member may be TGFβ3. The clonal progenitor cell line may be grown on or encased in a hydrogel, e.g. a hydrogel comprising thiolated hyaluronate, thiolated gelatin and/or both thiolated hyaluronate and thiolated gelatin.

In further embodiments the invention provides a method of obtaining a cell expressing UCP1 comprising contacting a clonal progenitor cell line with one or more TGFβ family members, optionally also contacting the clonal progenitor cell line with a PPARγ agonist, thereby obtaining a cell expressing UCP1. Suitable TGFβ family members include members of the BMP family, such as BMP4, BMP6. In some embodiments the TGFβ family member may be TGFβ3. The clonal progenitor cell line may be grown on or encased in a hydrogel, e.g. a hydrogel comprising thiolated hyaluronate, thiolated gelatin and/or both thiolated hyaluronate and thiolated gelatin.

In further embodiments the invention provides a method of obtaining a cell expressing C19orf80 comprising contacting a clonal progenitor cell line with one or more TGFβ family members thereby obtaining a cell expressing C19orf80. Suitable TGFβ family members include members of the BMP family, such as BMP4, BMP6, or BMP7. The clonal progenitor cell line may be grown on or encased in a hydrogel, e.g. a hydrogel comprising thiolated hyaluronate, thiolated gelatin and/or both thiolated hyaluronate and thiolated gelatin.

In still other embodiments the invention provides a method of obtaining a cell expressing one or more gene expression markers chosen from FABP4, C19orf80, ADIPOQ, or UCP1, comprising contacting a clonal progenitor cell line disclosed herein with a thiolated hyaluronate and thiolated gelatin-based hydrogel supplemented with 100 ng/ml BMP7, and 1.0 μM Rosiglitazone for 14 days wherein the cells are incubated for a period of time at lower than physiological temperature such as 28 C.

In still other embodiments the invention provides a method of obtaining a cell expressing one or more gene expression markers chosen from FABP4, C19orf80, ADIPOQ, or UCP1, comprising contacting a clonal progenitor cell line disclosed herein with a thiolated hyaluronate and thiolated gelatin-based hydrogel supplemented with 10 ng/ml BMP4, 1.0 μM rosiglitazone, 2.0 nM triiodothyronine (T3), and for the last 4 hours prior to use, 10 μM CL316243.

In yet other embodiments the invention provides a method of obtaining a cell expressing one or more gene expression markers chosen from FABP4, C19orf80, ADIPOQ, or UCP1, comprising contacting a clonal progenitor cell line disclosed herein with a thiolated hyaluronate and thiolated gelatin-based hydrogel supplemented with 100 ng/ml BMP7, and 5.0 μM Rosiglitazone for 14 days wherein the cells are incubated at a physiological temperature.

In yet other embodiments the invention provides a method of obtaining a cell expressing one or more gene expression markers chosen from FABP4, C19orf80, ADIPOQ, or UCP1, comprising contacting a clonal progenitor cell line disclosed herein with a thiolated hyaluronate and thiolated gelatin-based hydrogel supplemented with 1-50 ng/ml BMP4, and 1.0-5.0 μM Rosiglitazone for 14 days wherein the cells are incubated at a physiological temperature. In yet other embodiments the invention provides a method of obtaining a cell expressing one or more gene expression markers chosen from FABP4, C19orf80, ADIPOQ, or UCP1, comprising contacting a clonal progenitor cell line disclosed herein with a thiolated hyaluronate and thiolated gelatin-based hydrogel supplemented with 100 ng/ml BMP7, and 1.0-5.0 μM Rosiglitazone for 14 days wherein the cells are incubated for a final four hours in the presence of an added β3-specific adrenoceptor agonist (10 μM CL316243).

In other embodiments the invention provides a method of treating metabolic and vascular disease in a subject comprising administering to the subject one or more of the cells described infra. The metabolic or vascular disease may include Type I or Type II diabetes, syndrome X, obesity, hypertension, and atherosclerosis. The cells may be administered to the subject in a formulation comprised of cells in suspension in a physiologically-compatible salt solution or preferably in a matrix, most preferably a collagen and hyaluronic acid-based hydrogel as described infra.

Still other embodiments of the invention include kits and reagents comprising cells described herein and reagents useful for obtaining and/or growing the cells described herein.

### BRIEF DESCRIPTION OF THE DRAWINGS

FIG. 1. Is a graph showing microarray RFU values for the expression of FABP4 in selected FABP4+ samples exposed to one of the three adipocyte differentiation conditions tested. (RFU values>160RFU being considered positive).

FIG. 2. Is a graph showing microarray RFU values for the expression of C19ORF80 (also known as lipasin or betatrophin) in selected FABP4+ samples exposed to one of the adipocyte differentiation conditions tested. (RFU values>160RFU being considered positive).

FIG. 3. Is a graph showing microarray RFU values for the expression of ADIPOQ in selected FABP4+ samples exposed to one of the adipocyte differentiation conditions tested. (RFU values>160RFU being considered positive).

FIG. 4. Is a graph showing microarray RFU values for the expression of UCP1 in selected FABP4+ samples exposed to one of the adipocyte differentiation conditions tested. (RFU values>160RFU being considered positive).

FIG. 5. Is a graph showing microarray RFU values for the expression of PCK1 in selected FABP4+ samples exposed to one of the adipocyte differentiation conditions tested. (RFU values>160RFU being considered positive).

FIG. 6. Is a graph showing microarray RFU values for the expression of NNAT in selected FABP4+ samples exposed to one of the adipocyte differentiation conditions tested. (RFU values>160RFU being considered positive).

FIG. 7. Is a graph showing microarray RFU values for the expression of THRSP in selected FABP4+ samples exposed to one of the adipocyte differentiation conditions tested. (RFU values>160RFU being considered positive).

FIG. 8. Is a graph showing microarray RFU values for the expression of CEBPA in selected FABP4+ samples exposed to one of the adipocyte differentiation conditions tested. (RFU values>160RFU being considered positive).

FIG. 9. Is a graph showing microarray RFU values for the expression of CIDEA in selected FABP4+ samples exposed to one of the adipocyte differentiation conditions tested. (RFU values>160RFU being considered positive).

FIG. 10. Is a graph showing microarray RFU values for the expression of COX7A1 in selected FABP4+ samples exposed to one of the adipocyte differentiation conditions tested. (RFU values>160RFU being considered positive).

FIG. 11. Shows qRT-PCR data for the expression levels of the gene FABP4 in the clonal embryonic progenitor cell lines designated RAD20.5 and C4ELS5.1 in adipocyte differentiation conditions described herein.

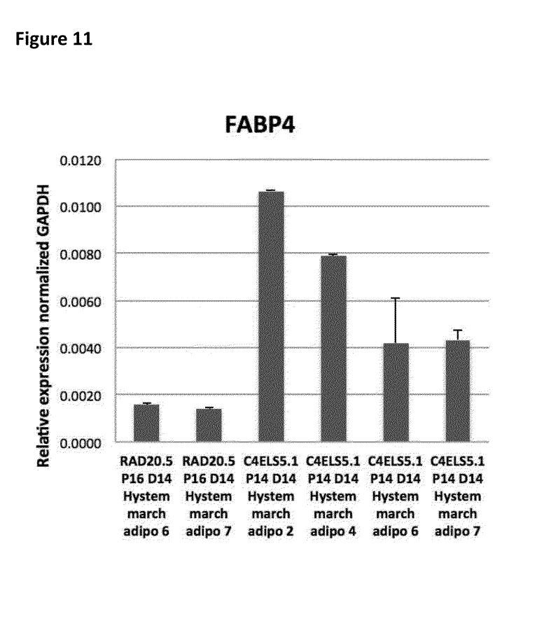

FIG. 12. Shows qRT-PCR data for the expression levels of the gene UCP1 in the clonal embryonic progenitor cell lines designated RAD20.5 and C4ELS5.1 in adipocyte differentiation conditions described herein.

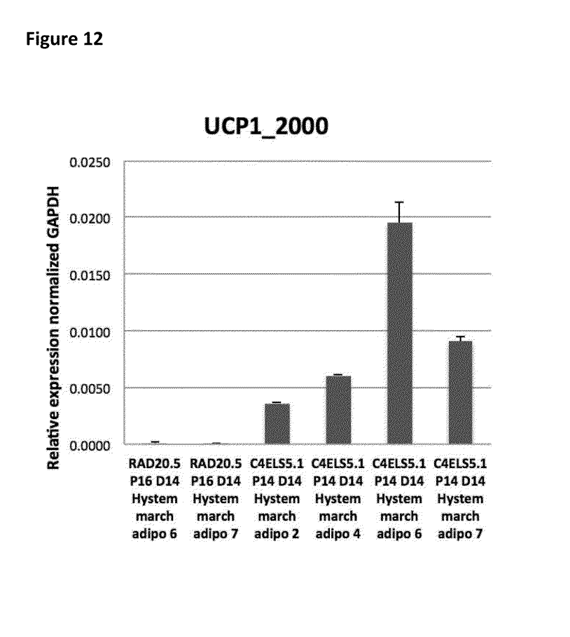

FIG. 13. Shows Illumina microarray-based data for the expression levels of the genes EYA4, MKX, FABP4, ELOVL3, UCP1, and ADIPOQ in the clonal embryonic progenitor cell lines designated C4ELS5.1 and E85 in the relatively undifferentiated progenitor state and then after 14 days of adipocyte differentiation conditions described herein designated March adipo 2 Adipocyte Differentiation Condition, March adipo 4 Adipocyte Differentiation Condition, March adipo 6 Adipocyte Differentiation Condition, and March adipo 7 Adipocyte Differentiation Conditions.

FIG. 14. Shows the expression of HOXA5 and IL13RA2 in clonal embryonic progenitor cell lines cultured in the relatively undifferentiated progenitor state after five days of quiescence where said clonal embryonic progenitors were derived under conditions to generate cells with a pattern of gene expression comparable to NP110SM and to be capable of differentiation into BAT cells simultaneously expressing UCP1, C19orf80, and ADIPOQ at levels comparable to isolated fetal-derived brown adipose tissue (fBAT) cells.

FIG. 15. Shows RFU values for the expression levels of the genes UCP1, C19orf80, ADIPOQ, and NTNG1 when fBAT cells and NP110SM cells are cultured for five days in reduced growth factors to induce quiescence in the progenitor state (labeled Ctrl) and when the cells are differentiated for 14 days in HyStem beads supplemented in 50 ng/ml BMP4 (lanes 2 and 6 respectively), 14 days supplemented with 5.0 μM rosiglitazone (lanes 3 and 7 respectively), 50 ng/ml BMP4 and 5.0 μM rosiglitazone (lanes 4 and 8 respectively), and when NP110SM is differentiated for 14 days supplemented with 50 ng/ml BMP4 and 5.0 μM rosiglitazone with the additional of 10 mM CL316243 for the four hours prior to harvesting RNA (lane 9).

FIG. 16. Shows qPCR results for the expression of the genes UCP1 and ADIPOQ in the novel EYA4+, ZIC2+ cell line C4ELSR2 in control conditions where the RNA was isolated from the cells in the quiescent progenitor state, and in diverse BAT cell differentiation conditions described herein, including Adipogenesis Protocol 3.

### DEFINITIONS AND ABBREVIATIONS

### Definitions

The term “adipose-derived SVF” refers to stromal vascular fraction cells from adipose tissue sources. Generally, these are liposuction material that is centrifuged to separate a pellet of cellular material (SVF) from less dense adipocytes. The adipose-derived SVF may also refer to such pelleted or otherwise liposuction-derived cells that are resuspended in liquid to be used in combination with the cells of the present invention as described herein.

The term “adult stem cells” refers to stem cells obtained from tissue originating from a mammal in stages of development after embryonic development is complete (in humans, this would refer to tissues of greater than eight weeks of gestational development). The tissue derived from the mammal may include tissue derived from a fetal or from an adult mammal and may include mesenchymal stem cells, neuronal stem cells, and bone marrow-derived stem cells.

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application No. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006).

The term “blastomere/morula cells” refers to blastomere or morula cells in a mammalian embryo, a mammalian in vitro fertilized egg, or blastomere or morula cells cultured in vitro with or without additional cells including differentiated derivatives of those cells.

For purposes of this disclosure, unless otherwise specified, the term “brown adipose cell” or “brown adipocyte” or “cellular component of brown adipose tissue (BAT)” refers to any cell that expresses adipocyte markers in conjunction with one or more of the genes UCP1, ADIPOQ or C19orf80 (also known as ANGPTL8 or LOC55908 [accession number NM_{—}018687.3, identified on Illumina gene expression microarrays as probe ID 1430689], encoding for the protein lipasin, also known as betatrophin). The term includes mature cells present in fetal or adult brown adipose tissue that express COX7A1, while the cells of the present invention do not express the mature marker COX7A1 but otherwise are functional brown adipose cells and are desirable for therapeutic use compared to fetal or adult-derived brown adipose cells due to a higher level of expression of neurite outgrowth promoting factors such as Netrin G1 expression (promoting innvervation of the tissue by the sympathetic nervous system) in the BAT cells produced from embryonic progenitor cells. The term also includes cells that are partially differentiated into brown adipocytes that express highest levels of Netrin G1 to promote said innvervation.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “clonal” refers to a population of cells obtained the expansion of a single cell into a population of cells all derived from that original single cells and not containing other cells.

The term “colony in situ differentiation” refers to the differentiation of colonies of cells (e.g., hES, hEG, hiPS, hEC or hED) in situ without removing or disaggregating the colonies from the culture vessel in which the colonies were propagated as undifferentiated stem cell lines. Colony in situ differentiation does not utilize the intermediate step of forming embryoid bodies, though embryoid body formation or other aggregation techniques such as the use of spinner culture may nevertheless follow a period of colony in situ differentiation.

The term “differentiated cells” when used in reference to cells made by methods of this invention from pluripotent stem cells refer to cells having reduced potential to differentiate all somatic cell types when compared to the parent pluripotent stem cells. By way of non-limiting example, human pluripotent stem cells such as hES cells are less differentiated than the hES-derived clonal embryonic progenitor cells of the present invention, which in turn are less differentiated than the in vitro produced brown fat progenitors of the present invention, which are less differentiated than fetal or adult-derived brown fat cells in that fetal or adult-derived brown fat cells that express COX7A1, a marker of cells in fetal or later stages of differentiation, and wherein the cells of the present invention do not yet express COX7A1. The differentiated cells of this invention comprise cells that may differentiate further (i.e., they may not be terminally differentiated).

The term “direct differentiation” refers to process of differentiating: blastomere cells, morula cells, ICM cells, ED cells, or somatic cells reprogrammed to an undifferentiated state (such as in the process of making iPS cells but before such cells have been purified in an undifferentiated state) directly without the intermediate state of propagating isolated undifferentiated stem cells such as hES cells as undifferentiated cell lines. A nonlimiting example of direct differentiation would be the culture of an intact human blastocyst into culture and the derivation of ED cells without the generation of a human ES cell line as was described (Bongso et al, 1994. Human Reproduction 9:2110).

The term “embryoid bodies” is a term of art synonymous with “aggregate bodies”, referring to aggregates of differentiated and undifferentiated cells that appear when pluripotent stem cells overgrow in monolayer cultures, or are maintained in suspension cultures. Embryoid bodies are a mixture of different cell types, typically from several germ layers, distinguishable by morphological criteria and cell markers detectable by immunocytochemistry. The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The blastocysts, blasotmeres, morulae and the like may be obtained from an in vitro fertilized egg. The ES cells may be derived from the in vitro fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

“Feeder cells” or “feeders” are terms used to describe cells of one type that are co-cultured with cells of another type, to provide an environment in which the cells of the second type can grow. Certain types of pluripotent stem cells can be supported by primary mouse embryonic fibroblasts, immortalized mouse embryonic fibroblasts, embryonic avian fibroblasts, or human fibroblast-like cells differentiated from hES cell. Pluripotent stem cell populations are said to be “essentially free” of feeder cells if the cells have been grown through at least one round after splitting in which fresh feeder cells are not added to support the growth of the cells.

A “growth environment” is an environment in which cells of interest will proliferate, differentiate, or mature in vitro. Features of the environment include the medium in which the cells are cultured, any growth factors or differentiation-inducing factors that may be present, and a supporting structure (such as a substrate on a solid surface) if present.

A cell is said to be “genetically altered”, “transfected”, or “genetically transformed” when a polynucleotide has been transferred into the cell by any suitable means of artificial manipulation, or where the cell is a progeny of the originally altered cell that has inherited the polynucleotide. The polynucleotide will often comprise a transcribable sequence encoding a protein of interest, which enables the cell to express the protein at an elevated level. The genetic alteration is said to be “inheritable” if progeny of the altered cell have the same alteration.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell lines (see, e.g., U.S. Pat. Nos. 7,582,479; 7,217,569; 6,887,706; 6,602,711; 6,280,718; and 5,843,780 to Thomson). The hED cells may be derived from preimplantation embryos produced by the in vitro fertilization of an egg cell with sperm or DNA, nuclear transfer, or chromatin transfer, an egg cell induced to form a parthenote through parthenogenesis, or analytical reprogramming technology.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application No. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US/00/29551, filed Oct. 27, 2000).

The term “human embryonic stem cells” (hES cells) refers to human ES cells which are lines of pluripotent stem cells generated from preimplantation human embryos, such as those discarded in the routine production of blastocysts in IVF procedures.

The term “human iPS cells” refers to cells with properties similar to hES cells, including the ability to form at least one cell type from all three germ layers (mesoderm, ectoderm and endoderm) when transplanted into immunocompromised mice wherein said iPS cells are derived from cells of varied somatic cell lineages following exposure to de-differentiation factors, for example hES cell-specific transcription factor combinations: KLF4, SOX2, MYC, and OCT4 or SOX2, OCT4, NANOG, and LIN28. Any convenient combination of de-differentiation factors may be used to produce iPS cells. Said iPS cells may be produced by the expression of these genes through vectors such as retroviral, lentiviral or adenoviral vectors as is known in the art, or through the introduction of the factors as proteins, e.g., by permeabilization or other technologies. For descriptions of such exemplary methods see: PCT application number PCT/US2006/030632, filed on Aug. 3, 2006; U.S. application Ser. No. 11/989,988; PCT Application PCT/US2000/018063, filed on Jun. 30, 2000; U.S. Application Ser. No. 09,736,268 filed on Dec. 15, 2000; U.S. Application Ser. No. 10/831,599, filed Apr. 23, 2004; and U.S. Patent Publication 20020142397 (application Ser. No. 10/015,824, entitled “Methods for Altering Cell Fate”); U.S. Patent Publication 20050014258 (application Ser. No. 10/910,156, entitled “Methods for Altering Cell Fate”); U.S. Patent Publication 20030046722 (application Ser. No. 10/032,191, entitled “Methods for cloning mammals using reprogrammed donor chromatin or donor cells”); and U.S. Patent Publication 20060212952 (application Ser. No. 11/439,788, entitled “Methods for cloning mammals using reprogrammed donor chromatin or donor cells”).

It will be appreciated that embryonic stem cells (such as hES cells), embryonic stem-cell like cells (such as iPS cells) and other pluripotent stem cells as well as progenitor cells derived from the cell types described infra may all be used according to the methods of the invention.

The term “ICM cells” refers to the cells of the inner cell mass of a mammalian embryo or the cells of the inner cell mass cultured in vitro with or without the surrounding trophectodermal cells. The ICM cells may be derived from an in vitro fertilized egg.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to mammalian cells capable of differentiating into more than one differentiated cell type of any of the three primary germ layers endoderm, mesoderm, and ectoderm including neural crest. Such cells include hES cells, blastomere/morula cells and their derived hED cells, hiPS cells, hEG cells, hEC cells. Pluripotent stem cells may be genetically modified or not genetically modified. By way on nonlimiting example, genetically modified cells may include markers such as fluorescent proteins to facilitate their identification when mixed with other cell types, or modifications of genes relating to immune surveillance to allow the cells to be tolerated allogeneically without rejection.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “primordial stem cells” which in this invention is used synonymously with “pluripotent stem cells” refers collectively to cells capable of differentiating into cells of all three primary germ layers: endoderm, mesoderm, and ectoderm, as well as neural crest. Human primordial stem cells therefore express stage-specific embryonic antigens (SSEA) SSEA3 and SSEA4, and markers detectable using antibodies designated Tra-1-60 and Tra-1-81. (Thomson et al., Science 282:1145, 1998) Therefore, examples of primordial stem cells would include but not be limited by human or non-human mammalian ES cells or cell lines, blastomere/morula cells and their derived ED cells, iPS, and EG cells, or the corresponding cells derived from parthenogenetic, gynogenetic, or nuclear transfer-derived embryos.

The term “universal donor cells” refers to cells derived from primordial stem cells that have been genetically modified to reduce immunogenicity through the modulation of expression of certain genes such as the knockout of one of both alleles of beta 2 microglobulin (B2M), knockout of HAL genes, or increased expression of HLA-G or HLA-H, or CTLA4-Ig and PD-L1, as well as other modifications enclosed herein or known in the art and subsequently used to generate differentiated cells for research and therapeutic applications wherein said cells have reduced immunogenicity

“Subject” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” “patient” or “animal” refers to a male. In some embodiments, the term “subject,” “patient” or “animal” refers to a female.

The terms “treat,” “treated,” or “treating” as used herein can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tissue regeneration” refers to at least partial regeneration, replacement, restoration, or regrowth of a tissue, organ, or other body structure, or portion thereof, following loss, damage, or degeneration, where said tissue regeneration but for the methods described in the present invention would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

“Adiponectin” or “ADIPOQ”, also known as AdipoQ, GBP-28, or apM1, is a protein that in humans is encoded by the ADIPOQ gene. Adiponectin modulates a number of metabolic processes, including glucose regulation and fatty acid oxidation. Adiponectin is secreted from adipose tissue into the bloodstream, where levels of the hormone are inversely correlated with body fat percentage, type II diabetes, and coronary disease. (Yamamoto et al, “Circulating adiponectin levels and risk of type 2 diabetes in the Japanese”, Nutr Diabetes. 2014 Aug. 18; 4:e130).

“FABP4 (fatty acid binding protein 4)”, also known as aP2 or AFABP, is a carrier protein for fatty acids that is primarily expressed in adipocytes and macrophages and encoded by the FABP4 gene in humans. Fatty acid binding proteins are a family of small, highly conserved, cytoplasmic proteins that bind long-chain fatty acids and other hydrophobic ligands. It is thought that FABPs roles include fatty acid uptake, transport, and metabolism. (Thumser et al, “Fatty acid binding proteins: tissue-specific functions in health and disease”, Curr Opin Clin Nutr Metab Care. 2014 March; 17(2):124-9).

“Lipasin”, also known as betatrophin or ANGPTL8, is a protein that in humans is encoded by the C19orf80 gene also known as LOC55908 (accession number NM_{—}018687.3, identified on Illumina gene expression microarrays as probe ID 1430689). C19orf80 is a putative peptide hormone that was found to increase the rate at which pancreatic beta cells undergo cell division in mice (Yi et al, Betatrophin: a hormone that controls pancreatic β cell proliferation, Cell. 2013 May 9; 153(4):747-58). Injection of mice with betatrophin cDNA resulted in lowered blood sugar levels, presumably due to action at the pancreatic islet cells. (Yi et al, Betatrophin: a hormone that controls pancreatic β cell proliferation, Cell. 2013 May 9; 153(4):747-58).

“UCP1 (uncoupling protein 1)”, also known as thermogenin or SLC25A7, is an uncoupling protein found in the mitochondria of brown adipose tissue and encoded by the UCP1 gene in humans. UCP1 is involved in heat generation heat by non-shivering thermogenesis (Golozoubova et al, Only UCP1 can mediate adaptive nonshivering thermogenesis in the cold, FASEB J. 2001, Sep. 15 (11):2048-50). UCP-1 uncouples oxidative phosphorylation from electron transport, yielding heat instead of ATP, as occurs in mitochondria without UCP-1 (Fedorenko et al, “Mechanism of fatty-acid dependent UCP1 uncoupling in brown fat mitochondria” Cell 2012 Oct. 12; 151(2)400-13). UCP1 is activated in brown fat cells by a signaling cascade initiated by release of norepinephrine by the sympathetic nervous system onto the Beta-3 adrenergic receptor on the plasma membrane (Cannon et al, “Brown adipose tissue function and physiological significance” Physiol Rev. 2004, 84, 277-359).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Abbreviations

BAT—Brown Adipose Tissue
BMP—Bone Morphogenetic Protein
cGMP—Current Good Manufacturing Processes
CNS—Central Nervous System
CT—Computed Tomography
CTLA4-Ig—Cytotoxic T lymphocyte antigen 4-immunoglobulin fusion protein
DMEM—Dulbecco's modified Eagle's medium
DMSO—Dimethyl sulphoxide
DPBS—Dulbecco's Phosphate Buffered Saline
EC—Embryonal carcinoma
EC Cells—Embryonal carcinoma cells; hEC cells are human embryonal carcinoma cells
ECAPCs—Embryonic Cutaneous Adipocyte Progenitor Cells
ECM—Extracellular Matrix
ED Cells—Embryo-derived cells; hED cells are human ED cells
EDTA—Ethylenediamine tetraacetic acid
EG Cells—Embryonic germ cells; hEG cells are human EG cells
ELBM—Embryonic Limb Bud Mesenchyme
ES Cells—Embryonic stem cells; hES cells are human ES cells. ES cells, including hES cells for the purposes of this invention may be in a naïve state corresponding to ICM cells of the human blastocyst, or the primed state corresponding to flattened epiblast cells (sometimes referred to as “ES-like” cells).
FACS—Fluorescence activated cell sorting
fBAT—fetal-derived brown adipose tissue
FBS—Fetal bovine serum
FDG—F18-fluorodeoxyglucose
GMP—Good Manufacturing Practices
H&E—Hematoxylin & Eosin
hED Cells—Human embryo-derived cells
hEG Cells—Human embryonic germ cells are stem cells derived from the primordial germ cells of fetal tissue.
hEP Cells—Human embryonic progenitor cells
hiPS Cells—Human induced pluripotent stem cells are cells with properties similar to hES cells obtained from somatic cells after exposure to hES-specific transcription factors such as SOX2, KLF4, OCT4, MYC, or the genes, RNAs, or proteins encoded by NANOG, LIN28, OCT4, and SOX2.
HSE—Human skin equivalents are mixtures of cells and biological or synthetic matrices manufactured for testing purposes or for therapeutic application in promoting wound repair.
ICM—Inner cell mass of the mammalian blastocyst-stage embryo.
iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2.
ITS—Insulin, transferrin, selenium
IVF—In vitro fertilization
MEM—Minimal essential medium
MSCs—Mesenchymal Stem Cells
NHACs—Normal human articular chondrocytes
NT—Nuclear Transfer
PBS—Phosphate buffered saline
PCR—Polymerase Chain Reaction
PD-L1—Programmed death ligand-1
PEGDA—Polyethylene glycol diacrylate
PET—Positron Emission Tomography
PNS—Peripheral Nervous System
qRT-PCR—quantitative real-time polymerase chain reaction
RFU—Relative Fluorescence Units
SCNT—Somatic Cell Nuclear Transfer
SFM—Serum-Free Medium
SVF—Stromal Vascular Fraction
TRF—Terminal Restriction Fragment; products of the digestion of telomeres following digestion with restriction endonucleases
WAT cells—White adipose tissue cells

### DETAILED DESCRIPTION

This invention solves the problem of generating large populations of highly purified cellular components of human brown adipose tissue by showing how to efficiently differentiate them from pluripotent stem cells.

Pluripotent stem (pPS) cells such as human induced pluripotent stem cells (hiPS) cells, human embryonic stem (hES) cells, and human parthenogenetic pluripotent stem cells (hPPS) cells and other cells with the potential of pluripotency, can be differentiated into normal functional cellular components of BAT on an industrial scale by first initiating general differentiation under certain defined conditions described herein, and then expanding clonal embryonic progenitor cells or pooled clonal embryonic progenitor cells, or oligoclonal embryonic progenitor cells that can simply be expanded in cell culture as adherent cells in traditional cell culture vessels or attached to beads in a slurry, cryopreserved, and expanded again, and then differentiated using the techniques described herein to generate cellular components of BAT useful for research and therapy. A strategy has been developed that helps optimize the combination of factors that are useful in the above-mentioned manufacturing technology. The techniques of this invention are oriented at producing a population of highly-enriched cells capable of differentiating into ADIPOQ, C19orf80, and UCP1-expressing BAT cells.

The development of methods to efficiently produce BAT cells from hES cells is important, because hES cells can be caused to proliferate indefinitely and can be genetically modified to allow the introduction of genetic modifications that allow the generation of off-the-shelf allogeneic cells that can then yield industrial scale manufacture of the desired therapeutically-useful differentiated cell type providing a means is known to manufacture said differentiated cell type with requisite standards of purity and identity. Accordingly, this invention provides a system that can be used to generate unbounded quantities of BAT cells.

The disclosure that follows provides a full description of how to make the BAT cells of this invention. It provides extensive illustrations of how these cells can be used in research and pharmaceutical development. The disclosure also provides pharmaceutical compositions, devices, and treatment methods for the use of pluripotent stem cell-derived BAT cells for regeneration and remodeling of BAT to restore youthful fat, lipoprotein, and glucose metabolism.

There is a growing need for improved methods of generating progenitor cell types from ES and iPS cells that display and maintain a uniform differentiated state, and exhibit site-specific homeobox gene expression. Adipocytes are an example of cell types with important site-specific differences in gene expression. The diverse types of adipocytes within the developed human body each have unique roles in maintaining physiological homeostasis. For example, subcutaneous fat differs in numerous aspects from visceral fat. In the case of subcutaneous fat, there are varieties of site-specific adipocytes that also differ from one another. While adipocytes in general provide a physiological function in storing energy for future metabolic needs, a specialized type of adipose tissue called brown adipose tissue (BAT) or simply “brown fat”, commonly restricted to the dorsal aspect of mammals such as between the scapulae in young mammals, or in the superclaviclar or cervical or region, differs in several respects from the white adipocytes in subcutaneous fat elsewhere in the body. Metabolically active BAT has been reported to be detectable in adult humans as assayed by PET/CT using as an imaging agent the glucose analog F18-fluorodeoxyglucose (FDG-PET/CT) (van der Lans et al, “Cold-activated brown adipose tissue in human adults: methodological issues” Am J Physiol Regul Integr Comp Physiol, 2014, Jul. 15; 307(2)R103-13)).

In the last 20 years, BAT has been discovered to function as a thermogenic as well as an organ that regulates energy, lipid, and lipoprotein metabolism. Brown fat cells are highly innervated by the sympathetic nervous system (SNS) and BAT thermogenesis is almost exclusively under SNS innervation control (Cannon et al, “Brown adipose tissue function and physiological significance” Physiol Rev. 2004, 84, 277-359). BAT can further function as an endocrine organ generating critical adipokines such as adiponectin (also known as AdipoQ, GBP-28 or aPM1, encoded in humans by the ADIPOQ gene,), and C19orf80 (also known betatrophin or ANGPTL8, encoded in humans by the C19orf80 gene) (Shehzad et al, “Adiponectin: regulation of its production and its role in human diseases” Hormone, 2012, Jan.-Mar., 11(1):8-20). It appears that the mitochondrial membrane protein UCP1 (uncoupling protein 1, also known as thermogenin, encoded in humans by the gene UCP1), expressed in certain cells resident in brown fat, is critical in the uncoupling of oxidative phosphorylation leading to thermogenesis by BAT (Fedorenko et al, “Mechanism of fatty-acid dependent UCP1 uncoupling in brown fat mitochondria” Cell 2012 Oct. 12; 151(2)400-13). Furthermore, there are two distinct types of brown fat cells, commonly designated as “brown” fat cells and “beige” fat cells. Brown and beige fat cells are reported to have different embryological origins wherein the brown fat cells are reported to be derived from MYF5+ progenitors also capable of skeletal muscle differentiation (Seale P, Bjork B, Yang W, et al., PRDM16 controls a brown fat/skeletal muscle switch. Nature (2008); 454:961-968), and beige fat cells are reported to be derived from MYF5-progenitors (Wu et al, “Beige adipocytes are a distinct type of thermogenic fat cell in mouse and human” Cell, Jul. 20, 2012, 150(2)366-376).

As used in the present invention, all cells of the present invention expressing UCP1 and one or more of ADIPOQ or C19orf80 are designated as “brown” fat cells. While small molecule drugs such as the thiazolidinedione class of compounds (rosiglitazone, also known as Avandia) have shown usefulness as antidiabetic agents, such compounds can often have serious side effects. Therefore, the concept of brown fat cell transplantation as a therapeutic regimen has emerged. Reports suggest that the loss of brown or beige fat cells may correlate with obesity, cardiovascular disease, hypertension, and type II diabetes and restoration of these cells by transplantation can reverse obesity and type II diabetes in nonhuman animal studies. There remains, however, a need for a method for the manufacture brown fat cellular components that express UCP1 and certain adipokines expressed by brown fat tissue such as adiponectin and C19orf80 on an industrial scale suitable for transplantation in humans for the treatment of these large and growing health problems.

Techniques such as the clonal propagation of human embryonic progenitor (hEP) cell lines may facilitate the derivation of purified and scalable cell lines corresponding to regional anlagen of diverse tissue types for use in research and therapy. In addition, the standardization of research around such defined and scalable progenitors may improve the reproducibility of differentiation studies from laboratory-to-laboratory.

The present invention teaches methods and compositions for the manufacture of specific cellular components of BAT tissue, including: 1) UCP1-expressing brown adipocytes that express low to undetectable levels of the adipokines adiponectin and betatrophin; 2) adiponectin+, betatrophin+ adipocytes that express low or no levels of UCP1; 3) UCP1-expressing brown adipocytes that express ADIPOQ and C19orf80 at levels comparable to fBAT cells, and 4) vascular endothelial cells expressing ITLN1 or ITLN2; and combinations of these three cell types with collagen and hyaluronic acid-based hydrogels with or without added cells from autologous adipose-derived SVF.

### Sources of Stem Cells

This invention can be practiced using stem cells of various types. Amongst the stem cells suitable for use in this invention are pluripotent stem cells derived from interchangeable sources all of which will perform as described herein. The pluripotent stem cells may be cells formed after activation of an oocyte, such as a blastocyst, or somatic cells reprogrammed by analytical reprogramming technology. Non-limiting examples are primary cultures or established lines of embryonic stem cells or embryonic germ cells, as exemplified below.

The techniques of this invention can also be implemented directly with primary embryonic or fetal tissue, deriving chondrocytes directly from primary cells that have the potential to give rise to chondrocytes without first establishing an undifferentiated cell line.

### Pluripotent Stem Cells

Mammalian pluripotent stem cells are capable of differentiating into more than one differentiated cell type of any of the three primary germ layers endoderm, mesoderm, and ectoderm including neural crest. For the purposes of the present invention, such cells include human induced pluripotent stem cells, human parthenogenetic stem cells derived from a parthenegenetically-activated oocyte (i.e. an egg cell activated without fertilization by a sperm cell), human embryonic stem cells, human embryonic germ cells derived from fetal genital ridges, and some human embryonal carcinoma cells. Pluripotent stem cells may be genetically modified or not genetically modified. By way on nonlimiting example, genetically modified cells may include markers such as fluorescent proteins to facilitate their identification when mixed with other cell types, or modifications of genes relating to immune surveillance to allow the cells to be tolerated allogeneically without rejection.

Embryonic stem cells can be isolated from blastocysts of members of the primate species (U.S. Pat. No. 5,843,780; Thomson et al., Proc. Natl. Acad. Sci. USA 92:7844, 1995) as well as from morula-staged embryos, and epiblast of the embryonic disc. Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000).

Briefly, excess human preimplantation embryos generated in the routine course of IVF procedures can be used, or one-cell human embryos can be expanded to the blastocyst stage (Bongso et al., Hum Reprod 4:706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 minutes three times in DMEM, and exposed to a 1:5 dilution of Guinea pig complement (Gibco) for 3 minutes (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mitotically-inactivated mouse, human, or avian fibroblast feeder layers.

After 9 to 15 days, the inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on feeder cells in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (.about.200 U/mL; Gibco) or by selection of individual colonies by micropipette. Clump sizes of about 50 to 100 cells are optimal.

### Reprogramming of Somatic Cells

Preparation of Reprogramming Medium:

Most media developed for human Embryonic Stem Cell (hESC) culture do not support RNA reprogramming, as the inclusion of cytokines from the TGFb superfamily can be inhibitory to reprogramming. Use of Pluriton Reprogramming Medium (Stemgent) is supportive of RNA reprogramming. Furthermore, conditioning the Pluriton Medium with reprogramming qualified human newborn foreskin fibroblasts (NUFFs-RQ from Global Stem) can increase the reprogramming efficiency. One week prior to initiating reprogramming, plate 2.5 million NUFFs per T175 tissue culture treated flask in DMEM with 10% Fetal Calf Serum (DMEM 10% FCS) and culture overnight in a 5% CO₂ normoxic incubator. The following day, replace the medium, rinse once with PBS and remove, and then overlay 25 ml of the Pluriton Medium on the irradiated NUFFs. Collect the medium and replenish each day with 25 ml of fresh Pluriton Medium for 5 days. Store the daily fractions at 4 C, pool and then filter through a 0.5μ low adherence filter to remove cellular debris. The conditioned Pluriton Medium can then be used or stored frozen until use.

Preparation of Human Fibroblasts for Reprogramming.

Human dermal fibroblasts derived from patient biopsy samples can readily be reprogrammed into iPS cells with RNA. Prior to initiating reprogramming plate, 2.5 million dermal fibroblasts per T175 tissue culture treated flask in DMEM with 10% Fetal Calf Serum (DMEM 10% FCS) and culture overnight in a 5% CO₂ normoxic (21% O₂) incubator. Allow the culture to grow to 80% confluence and then dissociate from the plate into a single-cell solution with 0.05% Trypsin EDTA solution. Inactivate the trypsin with DMEM 10% FCS, centrifuge, aspirate and re-suspend in DMEM 10% FCS medium at a density 25,000 cells per ml. Re-plate 50,000 target fibroblasts onto one well of a 6-well plate pre-coated with Matrigel (Corning) and culture overnight in a 5% CO₂ hypoxic (3-5% O₂) incubator overnight.

Transition of Human Fibroblasts to Reprogramming Medium:

Human dermal fibroblasts transfected with a cocktail of microRNAs are more receptive to subsequent reprogramming with mRNA encoding Oct4. Remove the DMEM 10% FCS medium, rinse once with PBS and then replace with 2 ml per well of conditioned Pluriton Medium supplemented with 300 ng/ml of recombinant B18R protein (eBioscience). Replace the plate in a 5% CO₂ hypoxic (3-5% O₂) incubator for at least 2 hours for the medium to equilibrate.

Transfection of Human Fibroblasts with RNAs:

Transfecting human dermal fibroblasts once with a cocktail of microRNAs prior to subsequent daily transfection with a cocktail of Oct4, Sox2, Klf4, c-Myc and Lin28 (OSKML) synthetic mRNA's can improve overall RNA reprogramming efficiency. To prepare the microRNA transfection complex, in two separate tubes add 3.5 μl of microRNA cocktail (Stemgent) to 21.5 μl of Stemfect buffer and 4 μl of Stemfect transfection reagent to 21 μl of Stemfect buffer. Combine the two and let stand for 15 minutes at room temperature. Add the 50 μl of microRNA transfection complex in a drop wise manner to one well of human fibroblasts in 2 ml of conditioned Pluriton Reprogramming Medium containing B18R. Swirl to mix and replace the plate in a 5% CO₂ hypoxic (3-5% O₂) incubator for overnight transfection. The following day, aspirate and replace with 2 ml of Reprogramming Medium containing B18R and proceed to transfect with the mRNA cocktail.

To prepare the mRNA transfection complex add 10 μl of the mRNA reprogramming cocktail (Stemgent) containing 1 μg of total mRNA from the (OSKML at a 3:1:1:1:1:1 ratio) to 15 μl of Stemfect buffer. In a separate tube, add 4 μl of Stemfect to 21 μl of Stemfect buffer. Combine the two and let stand for 15 minutes at room temperature. Add the 50 μl of mRNA transfection complex in a drop wise manner to one well of human fibroblasts in 2 ml of conditioned Pluriton Reprogramming Medium containing B18R. Swirl to mix and replace the plate in a 5% CO₂ hypoxic (3-5% O₂) incubator for overnight transfection.

Isolation of RNA-Reprogrammed iPS Cell Lines from Human Fibroblasts:

Repeat this daily transfection of mRNA for an additional 7 to 9 transfections. Stop once primary iPS colonies are obvious and change the medium to a defined, feeder-free medium such as E8 (LifeTechnologies). Primary colonies can be manually picked and passaged onto a feeder-free culture system such as E8 on Matrigel within 24-48 hours of the last transfection to expand clonal, stable RNA-reprogrammed iPS cell lines free of any viral or DNA contaminants. Further expansion and characterization can continue as for any human ES or IPS cell line to demonstrate karyotypic stability and retention of pluripotency.

### Propagation of Human Pluripotent Stem Cells in an Undifferentiated State

Human pluripotent stem cells such as iPS cells or hES cells can be propagated continuously in culture, using culture conditions that promote proliferation without promoting differentiation. Exemplary serum-containing ES medium is made with 80% DMEM (such as Knockout DMEM, Gibco), 20% of either defined fetal bovine serum (FBS, Hyclone) or serum replacement (WO 98/30679), 1% non-essential amino acids, 1 mM L-glutamine, and 0.1 mM beta-mercaptoethanol. Just before use, human bFGF is added to 4 ng/mL (WO 99/20741, Geron Corp.).

Traditionally, ES cells are cultured on a layer of feeder cells, typically fibroblasts derived from embryonic or fetal tissue. Said feeder cells can be of human, avian, or murine origin. In the case of murine, mouse embryos are harvested from a CF1 mouse at 13 days of pregnancy, transferred to 2.0 mL trypsin/EDTA, finely minced, and incubated 5.0 minutes at 37 degrees C. 10% FBS is added, debris is allowed to settle, and the cells are transferred to a tissue culture vessel with 90% DMEM, 10% FBS, and 2 mM glutamine. To prepare a feeder cell layer, cells are irradiated to inhibit proliferation but permit synthesis of factors that support ES cells (4000 rads gamma-irradiation). Culture plates are coated with 0.5% gelatin overnight, plated with 375,000 irradiated mouse embryonic fibroblasts per well, and used 5 hours to 4 days after plating. The medium is replaced with fresh hES medium just before seeding the human pluripotent stem cells.

Human pluripotent stem cells can also be maintained in an undifferentiated state even without feeder cells. The environment for feeder-free cultures includes a suitable culture substrate, particularly an extracellular matrix such as Matrigel, HyStem, or laminin. The pluripotent stem cells are plated at >15,000 cells/cm² (optimally 90,000 cells/cm² to 170,000 cells/cm²). Typically, enzymatic digestion is halted before cells become completely dispersed (approximately 5 min with collagenase IV). Clumps of about 10 to 2,000 cells are then plated directly onto the substrate without further dispersal. Alternatively, the cells can be harvested without enzymes before the plate reaches confluence by incubating the culture vessel about 5 min in a solution of 0.5 mM EDTA in PBS or mechanically aspirating colonies containing relatively undifferentiated cells with a small cytoplasmic to nuclear area ratio. After washing from the culture vessel, the cells are plated into a new culture without further dispersal. Feeder-free cultures are supported by a nutrient medium containing factors that support proliferation of the cells without differentiation. Such factors may be introduced into the medium by conditioning the medium with cells secreting such factors, such as irradiated (about 4,000 rad) primary mouse embryonic fibroblasts, telomerized mouse fibroblasts, or fibroblast-like cells derived from pluripotent stem cells. Medium can be conditioned by plating the feeders at a density of about 5-6×10⁴ cells/cm² in a serum free medium such as KO DMEM supplemented with 20% serum replacement and 4.0 ng/mL bFGF. Medium that has been conditioned for 1-2 days is supplemented with further bFGF, and used to support pluripotent stem cell culture for 1-2 days. Alternatively or in addition, other factors can be added that help support proliferation without differentiation, such as ligands for the FGF-2 or FGF-4 receptor, ligands for c-kit (such as stem cell factor), ligands for receptors associated with gp130, insulin, transferrin, lipids, cholesterol, nucleosides, pyruvate, and a reducing agent such as beta-mercaptoethanol. Features of the feeder-free culture method are further discussed in International Patent Publication WO 01/51616; and Xu et al., Nat. Biotechnol. 19:971, 2001.

Relatively undifferentiated pluripotent stem cells are desired and under the microscope they appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present on human embryonal carcinoma (hEC) cells. Differentiation of pluripotent stem cells in vitro results in the loss of SSEA-4, Tra-1-60, and Tra-1-81 expression, and increased expression of SSEA-1, which is also found on undifferentiated hEG cells.

Parental Cell Lines of the Invention and Characterization and Differentiation of the Same

Throughout the present invention, data is presented for specific human ES cell-derived clonal embryonic progenitor cell lines such as those designated herein as: E3, E72, E75, C4ELS5.1, C4ELSR2, and NP110SM. Compositions, methods, and uses described herein for the cells of the present invention apply to these identical cell lines at different passage levels, as well as pluripotent stem cell-derived clonal, pooled clonal, and oligoclonal embryonic progenitors with the same patterns of gene expression described herein, including said progenitor cell lines manufactured in clinical grade GMP-compatible manufacturing conditions.

The pluripotent stem cell-derived clonal embryonic progenitor cell line NP110SM was derived from the pluripotent stem cell line Envy (Costa et al, The hESC line Envy expresses high levels of GFP in all differentiated progeny, Nat Methods 2(4):259-260 (2005) and was expanded by serial trypsinization and passaging in standard cell culture vessels coated with gelatin as described herein to maintain the cells in a relatively undifferentiated progenitor state. The culture medium for said expansion was PromoCell Smooth Muscle Cell Medium 2 (Cat. No. 97064) or alternatively (MCDB131 medium) and growth supplement (Cat. No. 39267) obtained from PromoCell GmbH (Heidelberg, Germany). The supplement levels in the expansion medium are: 5% fetal calf serum, 0.5 ng/ml EGF, 2.0 ng/ml basic FGF, and 5.0 μg/ml insulin. The cells were then expanded in standard cell culture vessels coated with gelatin, and induced into quiescence by changing the media from the above-described expansion medium, to the same medium with 10% of the normal growth supplements provided by the supplier for five days. Therefore, the quiescence medium was PromoCell Smooth Muscle Cell Medium 2 (Cat. No. 97064) or alternatively (MCDB131 medium) and growth supplement (Cat. No. 39267) obtained from PromoCell GmbH (Heidelberg, Germany) at 10% normal concentrations recommended by the supplier, or 0.5% fetal calf serum, 0.05 ng/ml EGF, 0.2 ng/ml basic FGF, and 0.5 μg/ml insulin. When RNA was extracted from these NP110SM cells at passage 10 and induced into quiescence for 5 days a condition sometimes referred to as “control” or “Ctrl” herein, the cells displayed the following gene expression markers: DLK1, HOXA5, SLC7A14, NTNG1, HEPH, PGM5, IL13RA2, SLC1A3, and SBSN but unlike fetal or adult-derived BAT progenitors do not express COX7A1, or one or more markers chosen from POSTN, KRT34, MKX, HAND2, TBX15, HOXA10, PLXDC2, DHRS9, NNAT, and HOXD11.

The NP110SM cell line when differentiated for 14-21 days in conditions described herein to induce differentiation of the cells into BAT cells, induces the expression of general adipocyte markers such as FABP4 (accession number NM_{—}001442.1, Illumina Probe ID 150373), and CD36 (accession number NM_{—}000072.2, Illumina Probe ID 3310538), as well as BAT-specific markers such as UCP1, LIPASIN, and ADIPOQ at levels comparable or greater than cultured fBAT cells differentiated in the same conditions. The highest viability as measured by RNA yields and levels of the BAT markers observed in the presence of 50 ng/ml of BMP4, 5 μm rosiglitazone, and embedded in HyStem beads.

It is critical for BAT cells intended to be functionally engrafted in vivo is that the cells will recruit innervation by the sympathetic nervous system. The line NP110SM in the relatively undifferentiated progenitor state expressed abundant transcript for Netrin G1, also known as Axon Guidance Molecule. Netrin G1 (NTNG1) belongs to a conserved family of proteins that act as axon guidance cues during vertebrate nervous system development (Nakashiba et al., 2000 (PubMed 10964959). NP110SM expressed the transcript for the molecule at levels much higher than cultured fBAT cells demonstrating the value of clonal embryonic stem cell lines as a means of not only generating potent BAT cells that simultaneously express levels of UCP1, LIPASIN, and ADIPOQ transcript comparable to fBAT cells, but also express levels of transcript for NTNG1 that is higher than fBAT cells, and unlike fBAT cells, the line NP110SM does not express COX7A1 when cultured as progenitors or differentiated up to 21 days in vitro, a marker of cells differentiated at least to fetal or adult stages of development, thereby showing that NP110SM cells have novel properties useful for BAT cell research and cell-based therapy for diseases associated with a loss of BAT cells such as adiposity, hypertension, Type I and Type II diabetes, lipodystrophies, and coronary disease.

The cell line C4ELS5.1 at passage 12 was expanded by serial trypsinization and passaging in standard cell culture vessels coated with gelatin as described herein to maintain the cells in a relatively undifferentiated progenitor state. The cells were then induced into quiescence by changing the media from the propagation medium (EpiLife LSGS medium supplemented with growth factors as per manufacturer's conditions), to a medium with 10% of the normal levels of said growth factors, for five days to induce quiescence. When RNA was extracted from these P12 C4ELS5.1 cells induced into quiescence, the cells displayed the following gene expression markers: TAC1 (accession number NM_{—}013996.1, Illumina ID 6860594), EBF2 (accession number NM_{—}022659.2, Illumina ID 1030482), SCARA5 (accession number NM_{—}173833.4, Illumina ID 1030477), EYA4 (accession number NM_{—}004100.3, Illumina ID 1260180), and TBX1 (accession number NM_{—}005992.1, Illumina probe ID 4880730). The cells did not express HOXA10 (accession number NM_{—}153715.2, Illumina ID 3290427), ZIC2 (accession number NM_{—}007129.2, Illumina Probe ID 510368), or MKX (accession number NM_{—}173576.1, Illumina ID 6620017) when propagated in the relatively undifferentiated progenitor state.

The cell line C4ELSR2 at passage 12 was expanded by serial trypsinization and passaging in standard cell culture vessels coated with gelatin as described herein to maintain the cells in a relatively undifferentiated progenitor state. The cells were then induced into quiescence by changing the media from the propagation medium (EpiLife LSGS medium supplemented with growth factors as per manufacturer's conditions), to a medium with 10% of the normal levels of said growth factors, for five days to induce quiescence. When RNA was extracted from these P12 C4ELSR2 cells induced into quiescence, the cells displayed the following gene expression markers: EBF2 (accession number NM_{—}022659.2, Illumina ID 1030482), EYA4 (accession number NM_{—}004100.3, Illumina ID 1260180), but unlike the line C4ELS5.1, the line C4ELSR2 abundantly expressed the site-specific marker ZIC2 (accession number NM_{—}007129.2, Illumina Probe ID 510368). The C4ELSR2 cells did not express HOXA10 (accession number NM_{—}153715.2, Illumina ID 3290427), when propagated in the relatively undifferentiated progenitor state. The cells when cultured in BAT cell differentiation conditions described herein, express UCP1 and ADIPOQ, making the cells of interest to researchers in brown fat cell biology and potentially for cell-based therapy for metabolic diseases as described herein.

The cell line E72 at passage 11 was expanded by serial trypsinization and passaging in standard cell culture vessels coated with gelatin as described herein to maintain the cells in a relatively undifferentiated progenitor state. The cells were then expanded in standard cell culture vessels coated with gelatin, and induced into quiescence by changing the media from the propagation medium (DMEM supplemented with 20% FBS), to a medium with 10% of the normal levels of serum (in this case 2.0% FBS) for five days. When RNA was extracted from these E72 cells at passage 11 and induced into quiescence for 5 days, the cells displayed the following gene expression markers: HOXA10 (accession number NM_{—}153715.2, Illumina ID 3290427), POSTN (accession number NM_{—}006475.1, Illumina ID 510246), KRT34 (accession number NM_{—}021013.3, Illumina ID 3710168), MKX (accession number NM_{—}173576.1, Illumina ID 6620017), HAND2 (accession number NM_{—}021973.2, Illumina probe ID 4640563), the relatively rarely-expressed HOX gene HOXD11 (accession number NM_{—}021192.2, Illumina probe ID 5290142) implicated in forelimb development, and TBX15 (accession number NM_{—}152380.2, Illumina probe ID 6060113). The cells but did not express LHX8 (accession number NM_{—}001001933.1, Illumina ID 2900343), FOXF2 (accession number NM_{—}001452.1, Illumina ID 1660470), AJAP1 (accession number NM_{—}018836.3, Illumina ID 1300647), PLXDC2 (accession number NM_{—}032812.7, Illumina ID 5900497), ZIC2 (accession number NM_{—}007129.2, Illumina Probe ID 510368), or DLK1 (accession number NM_{—}003836.4, Illumina ID 6510259).

The line did not express relatively distal HOX genes such as HOXB7 (accession number NM_{—}004502.2, Illumina probe ID 2470328), and HOXC8 (accession number NM_{—}022658.3, Illumina probe ID 4640059) expressed by cultured MSCs from the iliac crest, or the HOX genes HOXC9, HOXC10, or HOXC11 expressed in hindlimb, but not forelimb bud mesenchyme. The expression of HOXA10 (a marker of forelimb and hindlimb bud mesenchyme, but the lack of many distal HOX genes such as HOXB7 or HOXC8, and the lack of expression of HOXC9, HOXC10, or HOXC11 provides evidence of the commitment of the cell line E72 or cells with the same gene expression markers of being forelimb bud mesenchyme.

When the cell line E72 at passage 11 was cultured for 21 days in HyStem with 10 ng/mL of TGFβ3 together with BMP4 (10 ng/mL), or alternatively, 21 days in HyStem with 10 ng/mL of TGFβ3 together with BMP2 (50 ng/mL), the differentiated cells expressed relatively high levels of markers of endochondral ossification including COL2A1, ALPL, IBSP, and osteopontin (SPP1), such expression of osteogenic markers being comparable to early passage differentiating cultured MSCs.

We observed that the culture of the line E72 at passage 11 in HyStem-C (BioTime, Inc. Alameda, Calif.) supplemented with 1.0 uM all-trans retinoic acid induced the expression of HOXB6, a marker of lateral plate mesenchyme. The lack of distal HOX gene expression such as HOXB7 or HOXC8, the expression of HOXA10 and HOXD11, and the inducibility of HOXB6 with retinoic acid provide evidence that the line E72 is mesodermal with potential to develop into forelimb bud mesenchyme. The line was unusual in that when differentiated in HyStem in the presence of BMP4 and TGFβ3 or BMP4 and TGFβ3 as described herein, the line expressed the markers normally associated with enamel, including enamelin (ENAM, accession number NM_{—}031889.1, Illumina probe ID 7160598) and amelogenin (AMELX, accession number NM_{—}001142.2, Illumina probe ID 5720730).

When the line E72 at passage 12 was cultured for 21 days in HyStem with 10 ng/mL of BMP4, the differentiated cells expressed relatively high levels of markers of adipocyte markers FABP4 and CD36. Such lateral plate mesoderm progenitors, especially those from the forelimb region, are useful in the production of brown fat cells and cells that expression C19orf80, a regulator of lipid metabolism and beta cell proliferation.

When differentiated in BMP4 and TGFβ3, the differentiated cells expressing osteogenic markers as well as markers of hard bone such as ENAM provide a useful and unique research model of osteogenesis and a scalable source of novel cells useful in the repair of bone (for conditions such as osteonecrosis, fractures, repair of bone following surgical resection of tumors, osteoporosis, and spinal vertebrae fusion). On the other hand, when differentiated in BMP4 only to yield C19orf80-expressing adipocytes, the differentiated cells provide for the scalable production of C19orf80-expressing brown fat cells useful in the regulation of lipids and beta cell proliferation, the latter being useful in the treatment of both type I and II diabetes. Levels of expression of markers of BAT cells such as betatrophin and adiponectin are further enhanced using the BAT cell differentiation conditions described herein. Other uses of these cells include drug screening to determine toxicity of drug compounds with respect to these progenitor cell lines. The cell lines may also be used to generate cDNA libraries to study gene expression in these progenitor cell types. The cDNA libraries may be used to compare the progenitor cell lines with their parental pluripotent stem cell or with a differentiated downstream derivative cell, i.e. a more differentiated cell type derived from the progenitor cell type. Additionally the cell lines may be used to generate antibodies against cell surface antigens expressed by the cell lines.

The cells may be formulated in hydrogels such as HyStem-C (BioTime, Inc. Alameda, Calif.) wherein the matrix is thiol-modified gelatin and thiolated hyaluronan crosslinked in vivo or in vitro with (polyethylene glycol diacrylate (PEGDA), or in alternative matrices or in solution without said matrices for research and therapeutic applications. For example the cells may be used in transplantation for the treatment of lipid disorders, such as for the treatment of hyperlipidemia or for the induction of beta cell proliferation as a therapeutic modality for type I or type II diabetes and formulated in HyStem-C (BioTime, Inc. Alameda, Calif.) and transplanted subcutaneously at dosages calculated to cause a therapeutically useful reduction in lipids or induction of beta cells and associated insulin.

The cell lines E75 and E163 at passage 11 and 12 respectively, displayed gene expression markers similar but slightly different from E72. The line E75 expressed the following genes: HOXA10 (accession number NM_{—}153715.2, Illumina ID 3290427) abundantly expressed in forelimb and hindlimb bud mesenchyme, POSTN (accession number NM_{—}006475.1, Illumina ID 510246), KRT34 (accession number NM_{—}021013.3, Illumina ID 3710168), MKX (accession number NM_{—}173576.1, Illumina ID 6620017), HAND2 (accession number NM_{—}021973.2, Illumina probe ID 4640563), the relatively rarely-expressed HOX gene HOXD11 (accession number NM_{—}021192.2, Illumina probe ID 5290142) implicated in forelimb development, and TBX15 (accession number NM_{—}152380.2, Illumina probe ID 6060113). Unlike the line E72 which did not express PLXDC2 (accession number NM_{—}032812.7, Illumina probe ID 5900497), the lines E75 and E163 did express PLXDC2. The line E75 did not express ZIC2 (accession number NM_{—}007129.2, Illumina Probe ID 510368), LHX8 (accession number NM_{—}001001933.1, Illumina ID 2900343), FOXF2 (accession number NM_{—}001452.1, Illumina ID 1660470), AJAP1 (accession number NM_{—}018836.3, Illumina ID 1300647), or DLK1 (accession number NM_{—}003836.4, Illumina ID 6510259), or the lateral plate mesoderm marker HOXB6 (Accession number NM_{—}018952.4, Illumina ID 6220189). In addition to not expressing HOXB6, the line did not express relatively distal HOX genes such as HOXB7 (accession number NM_{—}004502.2, Illumina probe ID 2470328), and HOXC8 (accession number NM_{—}022658.3, Illumina probe ID 4640059) expressed by cultured MSCs from the iliac crest, or the HOX genes HOXC9, HOXC10, or HOXC11 expressed in hindlimb, but not forelimb bud mesenchyme.

When the cell line E75 at passage 11 was cultured for 21 days in HyStem with 10 ng/mL of TGFβ3 together with BMP4 (10 ng/mL), or alternatively, 21 days in HyStem with 10 ng/mL of TGFβ3 together with BMP2 (50 ng/mL), the differentiated cells expressed relatively high levels of markers of endochondral ossification including COL2A1, ALPL, IBSP, and osteopontin (SPP1), such expression of osteogenic markers being comparable to early passage differentiating cultured MSCs. In addition, and the line was unusual in that when differentiated in HyStem in the presence of BMP4 and TGFβ3 or BMP4 and TGFβ3 as described herein, the line expressed the markers normally associated with enamel, including enamelin (ENAM, accession number NM_{—}031889.1, Illumina probe ID 7160598) and amelogenin (AMELX, accession number NM_{—}001142.2, Illumina probe ID). Unlike the line E72 described herein, the line E75 in differentiated for 21 days in the presence of HyStem and BMP4 and TGFβ3 as described herein, expressed the additional marker normally associated with enamel called ameloblastin (AMBN) (accession number NM_{—}016519.4, Illumina probe ID 6400438).

When the lines E75 and E163 at passages 11 and 12 respectively were cultured for 21 days in HyStem with 10 ng/mL of BMP4, the differentiated cells expressed relatively high levels of adipocyte markers FABP4 and CD36 and candidate BAT cell progenitor markers such as C19orf80. Such lateral plate mesoderm progenitors, especially those from the forelimb region, and especially when differentiated in the BAT cell differentiation conditions described herein, are useful in the production of brown fat cells and cells that express C19orf80, a regulator of lipid metabolism and beta cell proliferation.

The differentiated cells from the parental cells lines E75 and E163 provide useful and unique research models for osteogenesis or adipogenesis depending on the differentiation protocol used. The differentiated cells expressing osteogenic markers, as well as markers of hard bone such as are present in the enamel of teeth, provide a model of osteogenesis as well a scalable source of novel cells useful in the repair of bone. The cells may find use in treating conditions of osteonecrosis, fractures, repair of bone following surgical resection of tumors, osteoporosis, and spinal vertebrae fusion. The cells expressing the markers of adipocyte differentiation from the interscapular region of the back, provide a model of adipogenesis, in particular of C19orf80-secreting adipocytes, as well for the scalable production of brown fat cells and cells capable of secreting C19orf80 useful in the regulation of lipids and beta cell proliferation, the latter being useful in the treatment of both type I and II diabetes. Specific differentiation conditions may be used to obtain cells expressing adipocyte markers as opposed to cells expressing osteogenic markers.

The cells may be formulated in hydrogels such as HyStem-C (BioTime, Inc. Alameda, Calif.) wherein the matrix is thiol-modified gelatin and thiolated hyaluronan crosslinked in vivo or in vitro with (polyethylene glycol diacrylate (PEGDA), or in alternative matrices or in solution without said matrices for research and therapeutic applications. For example for transplantation of cells for the treatment of lipid disorders, such as for the treatment of hyperlipidemia or for the induction of beta cell proliferation as a therapeutic modality for type I or type II diabetes, the cells may be formulated in HyStem-C (BioTime, Inc. Alameda, Calif.) and transplanted subcutaneously at dosages calculated to cause a therapeutically useful reduction in lipids or induction of beta cells and associated insulin.

Parental and Progeny Cell Lines with Reduced Immunogenicity

In various embodiments of the present invention, several methodologies for reducing immunogenicity of the parental and progeny cell lines in order to reduce allogenic rejection could be used prior or after cell differentiation. Ideally, shielding the donor line from any immune rejection could give rise to a universal donor parental cell lines, reducing or eliminating the need for toxic immune rejection prevention drugs in the recipient.

Human leukocyte antigen (HLA) molecules are cell surface proteins present in most cells. They are highly polymorphic and used to present antigens to the immune system to fight disease and eliminate foreign bodies. Class I HLA are heterodimers composed of two main subunits, the macroglobulin (B2M) which is essential for cell surface expression, and the HLA class I heavy chain subunit HLA-A, B, C, E, F, or G. Class I HLA-A, B and C are the major histocompatibility determinants and need to be matched between donors. Class I HLA molecules are important determinants presenting foreign peptides to activate cytotoxic T cells to destroy foreign cells. HLA class II proteins (HLA-DP, DQ and DR) are used by antigen-presenting cells and stimulate the production of antibodies by B Cells to eliminate foreign antigens.

Several methodologies can be used to minimize or eliminate the modulation of the recipient's immune system in response to donor cells. The first one consists in matching donor HLA types with the recipient HLA-type. In one embodiment of the present invention, a series of parental cell lines consisting in a library of pure selected HLA-types representing all or a subset of all possible HLA variants could be used. This would require the generation of a large library of clonally derived cells with representing various HLA types, each clonal line matching a group of recipients with corresponding HLA type.

An alternative approach consists in the removal of HLA alleles, which are responsible for the presentation foreign body antigens to the immune system. Individual HLA alleles could be knocked out individually, or by knocking out the Beta-2 microglobulin (B2M) which is common to all HLA class I and necessary for cell surface expression (Riolobos, L., et al. HLA engineering of human pluripotent stem cells. Mol Ther 21, 1232-1241 (2013). HLA class II molecules can be suppressed by knocking out the essential transcription factor gene RFXANK necessary for their expression (DeSandro, A. M., Nagarajan, U. M. and Boss, J. M. Associations and interactions between bare lymphocyte syndrome factors. Mol Cell Biol 20, 6587-6599 (2000). To avoid the potential destruction of class I-negative cells by Natural Killer (NK) cells, donor cells can be engineered to expressed a non-polymorphic HLA molecule (HLA-E) which has been demonstrated to inhibit NK cell activation (Lee, N., et al. HLA-E is a major ligand for the natural killer inhibitory receptor CD94/NKG2A. Proc. Natl. Acad Sci USA 95, 5199-5204 (1998).

Several techniques well-known in the art can be used for knocking out/editing various HLA allele and would be evident for people skilled in the art. They may include various DNA vectors, naked or encapsulated in a carrier virus or liposomes used to specifically replace or edit various genes by homologous recombination. Alternatively, other expression knock out approaches such as siRNA, anti-sense oligonucleotides could be used alone or in combination with other knock out approaches.

Another methodology, which could be employed to reduce immunogenicity of donor cells consists in engineering the cells to overexpressed HLA-G. HLA-G is expressed by placental cells during pregnancy and confers immunosuppressive properties. HLA-G-modified cells and methods are described in the PCT/US2013/05757 patent application.

Other immune modulation strategies could also be employed in various embodiments of the present invention in order to maximize long-term engraftment of the donor cells in an allogenic recipient. Methodologies that aim at repressing the T-cell activation have been used successfully by Rong et al (An effective approach to prevent immune rejection of human ESC-derived allografts. Cell Stem Cell 14, 121-132 (2008)) to reduce rejection in a mouse model of human allogenic transplantation. Their approach consists in over expressing the Cytotoxic T-Lymphocyte Antigen 4 (CTLA4) and the Programmed Cell Death Ligand 1 (PD-L1), which are known to inhibit T-Cell activation.

In certain embodiments of the present invention when long term engraftment is important to provide a long term clinical benefit, donor cells which could evade or repress immune rejection would be a distinct advantage. Moreover, in a preferred embodiment of the present invention, a universal parental pluripotent stem cell line, such as a human iPS cell or ES cell line engineered by the methods described herein alleviate the need to develop multiple progeny lines matching various genetic background of different recipient, or the recourse to toxic immune suppression drugs.

Progenitor Cells that can Give Rise to Brown Fat Cells

In various embodiments described infra the invention provides progenitor cells, e.g. isolated progenitor cell lines that give rise to brown fat cell types. In some embodiments the progenitor cells are capable of differentiation into cells that express one or more markers expressed by various brown fat cells. Exemplary markers expressed by any particular brown fat cell types of the present invention include one or more of the following: FABP4, C19orf80, ADIPOQ, UCP1, PCK1, NNAT, THRSP, CEBPA, CIDEA. In fully mature cells following transplantation into humans corresponding to in vivo-derived brown fat cells from adult or fetal-sources, COX7A1 is expressed. However, COX7A1 is not expressed in the progenitor cells of the present invention or in the brown fat cellular components derived from said progenitors in vitro prior to transplantation in vivo, reflecting that the brown fat cells of the present invention are in a primitive state of differentiation corresponding to the embryonic as opposed to fetal or adult stages of differentiation and have not previously been described in the art.

In some embodiments the invention provides an isolated cell line expressing C19orf80. In some embodiments the invention provides an isolated progenitor cell line expressing UCP1. In some embodiments the invention provides a combined formulation of cells expressing C19orf80 and UCP1.

The isolated progenitor cell line, e.g. the isolated progenitor cell line that gives rise to brown fat cells may be the in vitro differentiated progeny of a pluripotent stem cell. The brown fat cells can be obtained by differentiating the isolated progenitor cell line under suitable culture conditions described infra. Accordingly, brown fat cells of the invention may have essentially the same genome as the parental cell from which it was derived. The parental cell may be the progenitor cell line described infra, or a pluripotent precursor the progenitor cell described infra. Examples of pluripotent precursors of the progenitor cells described infra include ES cells such as hES cells, iPS such as human iPS cells and the like. Thus in some embodiments the brown fat cells of the invention may have a genome that is about 95%, 96%, 97%, 98%, 99% identical to its pluripotent parental cell or cell line. In some embodiments of the invention the brown fat cells of the invention will have a genome that is greater than 90%, greater than 93%, greater than 94%, greater than 95%, greater than 96%, greater than 97%, greater than 98%, greater than 99% identical to its pluripotent parental cell or cell line.

### Progenitor Cell Lines

Progenitor cells and progenitor cell lines are used interchangeably herein and refer to cultures of cells that can be propagated for at least 5 passages, but nevertheless are mortal and eventually senesce due to telomere shortening.

Certain embodiments of the invention provide progenitor cell lines, methods of making progenitor cell lines and methods of using progenitor cell lines. Progenitor cell lines may, in some embodiments, be the progeny, such as the in vitro progeny, of an embryonic stem cell(s) (e.g. an ES cell(s) such as a hES cell(s)) or an iPS cell(s). The ES cell or iPS cell(s) may be obtained from a mammal, such as a primate. In one embodiment the ES or iPS cell(s) is of human origin. The progenitor cell(s) may be obtained from an established ES cell line available from cell bank, such as WiCell or BioTime, Inc. The progenitor cell may be obtained from ES cell line generated without destroying an embryo or an in vitro fertilized egg (Chung et al. Cell Stem Cell (2008) 2:113).

Progenitor cells may include clonal or oligo-clonal progenitor cell lines. Progenitor cells may have the ability to replicate in culture through multiple passages. In some embodiments of the invention the progenitor cells may be passaged about 1-100 times, about 5-90 times, about 10-80 times, about 20-70 times, about 30-60 times, about 40-50 times. In some embodiments the progenitor cells may be passaged about 5 times, about 10 times, about 11 times, about 12 times, about 13 times, about 14 times, about 15 times, about 16 times, about 17 times, about 18 times, about 19 times, about 20 times, about 21 times, about 22 times, about 23 times, about 24 times, about 25 times.

In certain embodiments the invention provides progenitor cell lines that have the ability to differentiate into cells found in an animal body, such as a human. Differentiation may be induced for example, by altering the culture conditions in which the progenitor cells are typically maintained. For example, growth factors, cytokines, mitogens or the like may be added or removed from the culture media.

In some embodiments the progenitor cells are multipotent cells. In some embodiments the progenitor cells are not pluripotent cells. In some embodiments the progenitor cells are not mesenchymal stem cells (MSC). In some embodiments the progenitor cells do not express one or more markers found on a mesenchymal stem cell. In some embodiments the progenitor cells express one or more markers found on an MSC at level that is lower than the expression level found on an MSC. In some embodiments of the invention the progenitor cells do not express CD74. In some embodiments of the invention the progenitor cells express CD74 at level that is lower than the level found on an MSC. In some embodiments the progenitor cell lines express one or more genes expressed by a chondrocyte or a chondrocyte precursor.

In certain embodiments the invention provides a progenitor cell chosen from the cell lines designated C4ELSR2, C4ELS5.1, E3, E72, E75, E163 and NP110SM.

In some embodiments the invention provide a progenitor cell line with a pattern of gene expression of the cell line C4ELSR2 that when clonally expanded from cultures of differentiating hES cells until a single cells has proliferated to the point of confluence in a 50 mm tissue culture dish (considered passage 1) and then expanded to passage 12 and induced into quiescence for 5 days as described herein, the cells displayed the gene expression markers: EBF2 (accession number NM_{—}022659.2, Illumina ID 1030482), EYA4 (accession number NM_{—}004100.3, Illumina ID 1260180), but unlike the line C4ELS5.1, the line C4ELSR2 abundantly expressed the site-specific marker ZIC2 (accession number NM_{—}007129.2, Illumina Probe ID 510368). The C4ELSR2 cells did not express HOXA10 (accession number NM_{—}153715.2, Illumina ID 3290427), when propagated in the relatively undifferentiated progenitor state.

In some embodiments the invention provide a progenitor cell line with a pattern of gene expression of the cell line C4ELS5.1 that when clonally expanded from cultures of differentiating hES cells until a single cells has proliferated to the point of confluence in a 50 mm tissue culture dish (considered passage 1) and then expanded to passage 12 and induced into quiescence for 5 days as described herein, the cells displayed the gene expression markers: TAC1 (accession number NM_{—}013996.1, Illumina ID 6860594), EBF2 (accession number NM_{—}022659.2, Illumina ID 1030482), SCARA5 (accession number NM_{—}173833.4, Illumina ID 1030477), EYA4 (accession number NM_{—}004100.3, Illumina ID 1260180), TBX1 (accession number NM_{—}005992.1, Illumina probe ID 4880730), but did not express HOXA10 (accession number NM_{—}153715.2, Illumina ID 3290427) or MKX (accession number NM_{—}173576.1, Illumina ID 6620017) when propagated in the relatively undifferentiated progenitor state.

In yet other embodiments the invention provides a progenitor cell line with a pattern of gene expression of the cell line E3 that expresses one or more genes chosen from POSTN, KRT34, MKX, HAND2, TBX15, HOXA10, PLXDC2, DHRS9, NNAT, and HOXD11. In certain embodiments the progenitor cell lines of the invention do not express one or more genes chosen from LHX8, FOXF2, AJAP1, DLK1, SLC7A14, NTNG1, and SFRP1. The progenitor cell line may have the potential to differentiate into a chondrocyte or adipocyte, or precursor thereof.

In yet other embodiments the invention provides a progenitor cell line with a pattern of gene expression of the cell line E72 expressing one or more genes chosen from POSTN, KRT34, MKX, HAND2, TBX15, DHRS9, NNAT, HOXA10, and HOXD11. In certain embodiments the progenitor cell lines of the invention do not express one or more genes chosen from FOXF2, AJAP1, PLXDC2, DLK1, HOXB6, HOXB7, HOXC8, SLC7A14, NTNG1, and SFRP1. The progenitor cell line may have the potential to differentiate into a chondrocyte or adipocyte.

In yet other embodiments the invention provide a progenitor cell line with a pattern of gene expression of the cell line E75 that expresses one or more genes chosen from POSTN, KRT34, MKX, HAND2, TBX15, HOXA10, PLXDC2, NNAT, and HOXD11. In certain embodiments the progenitor cell lines of the invention with a pattern of gene expression of the cell line E75 do not express one or more genes chosen from LHX8, FOXF2, AJAP1, DHRS9, DLK1, SLC7A14, NTNG1, and SFRP1. The progenitor cell line may have the potential to differentiate into a chondrocyte or adipocyte.

In still other embodiments the invention provides a progenitor cell line with a pattern of gene expression of the cell line E163 that expresses one or more genes chosen from: POSTN, KRT34, MKX, HAND2, TBX15, DHRS9, HOXA10, PLXDC2, NNAT, and HOXD11. In certain embodiments the progenitor cell lines of the invention with a pattern of gene expression of the cell line E163 do not express one or more genes chosen from LHX8, FOXF2, AJAP1, DLK1 and NTNG1. The progenitor cell line may have the potential to differentiate into a chondrocyte or adipocyte.

In yet other embodiments the invention provides a progenitor cell line with a pattern of gene expression of the cell line NP110SM expressing one or more genes chosen from: DLK1 (accession number NM_{—}003836.4, Illumina ID 6510259), HOXA5 (accession number NM_{—}019102.2, Illumina ID 6620437), SLC7A14 (accession number NM_{—}020949.1, Illumina ID 6100717), NTNG1 (accession number NM_{—}014917.2, Illumina ID 6940053), HEPH (accession number NM_{—}138737.1, Illumina ID 1850349), PGM5 (accession number NM_{—}021965.3, Illumina ID 4480112), IL13RA2 (accession number NM_{—}000640.2, Illumina ID 5420386), SLC1A3 (accession number NM_{—}004172.3, Illumina ID 4210403), and SBSN (accession number NM_{—}198538.1, Illumina ID 4480477). In some embodiments the invention provides a progenitor cell with a pattern of gene expression of the cell line NP110SM that does not express one or more genes chosen from MKX (accession number NM_{—}173576.1, Illumina ID 6620017), NNAT (accession number NM_{—}181689.1, Illumina ID 4010709), HOXD11 (accession number NM_{—}021192.2, Illumina ID 5290142), and DHRS9 (accession number NM_{—}005771.3, Illumina ID 630315). The progenitor cell line has the potential to differentiate into a population of highly purified brown adipocytes that simultaneously express relatively high levels of the BAT gene expression marker UCP1 as well as express relatively high levels the gene expression markers LOC55908 (TD26, betatrophin, C19orf80) (accession number NM_{—}018687.5, Illumina ID 1430689), CIDEC (accession number NM_{—}022094.2, Illumina ID 780309), UCP2 (accession number NM_{—}003355.2, Illumina ID 6580059), ELOVL6 (accession number NM_{—}024090.1, Illumina ID 5670040), (accession number, Illumina ID), CKMT1A (accession number NM_{—}001015001.1, Illumina ID 3420661), and ADIPOQ (accession number NM_{—}004797.2, Illumina ID 4200471), similar to cultured human fetal BAT-derived cells induced to differentiate into brown adipocytes, but unlike said human fetal BAT-derived cells in the preadipocyte or differentiated adipocyte state, the brown adipocytes derived from said hES cell-derived clonal embryonic progenitor cell line designated NP110SM does not express COX7A1 in either the undifferentiated or differentiated states, and when said hES cell-derived clonal embryonic progenitor cell line designated NP110SM is differentiated for 14 days into brown adipocytes, the cells express relatively low or no detectable expression of the gene expression marker CIDEA (accession number NM_{—}001279.2, Illumina ID 10048) unlike cultured human fetal BAT-derived cells which induce relatively high levels of expression of CIDEA when said human fetal BAT-derived cells are induced to differentiate into brown adipocytes.

In certain embodiments, adult-derived cells may be useful in the manufacture of BAT cells for research and therapy. Arterial smooth muscle cells such as coronary smooth muscle cells that are derived from individuals exposed to high levels of circulating ketone bodies such as may be present in individuals with significant long-term alcohol intake are capable of BAT cell differentiation using the methods disclosed herein. In addition, said smooth muscle cells capable of BAT cell differentiation may be transiently or permanently immortalized through the exogenous expression of the catalytic component of telomerase (TERT), thereby allowing the industrial expansion of said progenitors to BAT cells. Similarly, fetal or adult BAT tissue-derived preadipocytes may be transiently or permanently immortalized through the exogenous expression of the catalytic component of telomerase (TERT), thereby allowing the industrial expansion of said progenitors to BAT cells.

Any of the progenitor cell lines described infra may be used in the methods described infra. For example the progenitor cell lines may be contacted with a member of the TGF-β superfamily and induced to differentiate. The progenitor cell lines described infra may be contacted with retinoic acid and induced to differentiate. The progenitor cell lines described infra may be contacted with an agonist or antagonist of PPARγ and induced to differentiate. The progenitor cell lines described infra may be contacted with a thyroid hormone such as T3 or T4 and induced to differentiate. The progenitor cell lines described infra may be contacted with an adrenergic hormone such as epinephrine or norepinephrine and induced to differentiate. The progenitor cell lines described infra may be incubated at temperatures substantially below 37 deg C and induced to differentiate. The progenitor cell line may be cultured in a hydrogel at temperatures substantially below normal body temperature, as described infra, with or without a differentiation agent such as a member of the TGF-β superfamily, retinoic acid, agonist of PPARγ, adrenergic agonist, and thyroid hormone.

To improve the scalability of purified somatic progenitors from hPS cells, we previously reported the generation of a library of >140 diverse clonal human embryonic progenitor (hEP) cell lines as source of purified cell types with site-specific homeobox gene expression. We designated these novel cell lines “embryonic progenitors” because they show the potential to be propagated extensively in vitro and can subsequently differentiate in response to diverse growth factors and inducers. The term therefore refers to cells with an intermediate differentiated state between pluripotent cells and terminally differentiated cell types.

After screening 100 diverse hEP lines for chondrogenic potential, we identified seven lines that showed the induction of the chondrocyte marker COL2A1. One of these lines, 4D20.8, showed expression of site-specific craniofacial mesenchyme markers such as LHX8 and BARX1. We demonstrated long-term scalability of 4D20.8 in the undifferentiated state, and an ability to regenerate bone and cartilage when engrafted in articular defects in rat models. Subsequently the cell line 4D20.8 was compared with six other diverse osteochondral progenitor lines each of which showed site-specific homeobox gene expression markers as well as diverse phenotypes when differentiated in the presence of one or more TFG-beta superfamily members, such as, TGFβ3, BMP2, 4, 6, and 7, and GDF5.

In certain embodiments disclosed herein, the comparative site-specific gene expression of clonal embryonic progenitor cell lines capable of differentiating into site-specific adipocytes with patterns of gene expression useful in generating brown fat cells is provided and along with the disclosure of their diverse responses when differentiated in the presence of one or more TGF-beta superfamily members, such as, TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal.

In still other embodiments the invention provides a cell culture comprising the progenitor cell lines C4ELSR2, C4ELS5.1, E3, E72, E75, E163 and NP110SM or cell lines with a pattern of gene expression of C4ELSR2, C4ELS5.1, E3, E72, E75, E163 and NP110SM as described herein, cultured in micromass or cultured in a hydrogel. The cell culture may comprise one or more TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal.

Therefore, the present invention describes a composition comprising a first and a second cell population, wherein the first cell population comprises the relatively undifferentiated clonal, pooled clonal, or oligoclonal embryonic progenitor cells from which the second population is derived, and the second population comprises the in vitro differentiated progeny of the first cell population, wherein the cells of the second cell population express FABP4 and either UCP1, C19orf80, or ADIPOQ at levels comparable to cultured fBAT cells.

### Clonal Embryonic Vascular Endothelial Cells Expressing ITLN1 or ITLN2

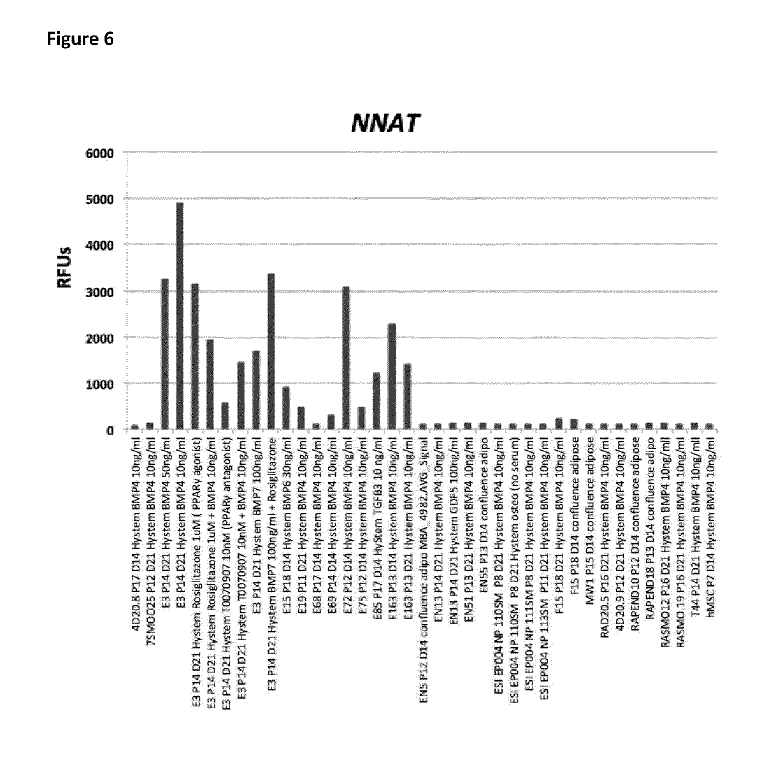

In another embodiment, pluripotent stem cells such as hES or iPS cells are differentiated in vitro to generate vascular endothelial cells that express omentin 1 (ITLN1) or intelectin-2 (ITLN2) and used in combination with the SVF, hydrogels, or the brown fat progenitors of the present invention. Said ITLN1 or ITLN2-expressing endothelial cells are generated in the presence of Activin-A and WNT-3A followed by FGF-4 and BMP-2 and then cloned as monoclonal cell lineages on Matrigel, gelatin, or similar supportive culture support in the presence of media capable of supporting the growth of vascular endothelial cells. More specifically, hES or iPS cells are cultured as colonies on fibroblast feeder cells that are allowed to overgrow and differentiate in situ for 13 days in ES cell culture medium such as Invitrogen KO-DMEM with KO-serum replacement. Then, on differentiation day 0 (FIG. 6), media is changed to a basal differentiation media comprising KO-DMEM/RPMI-1640 (5/1 v/v) and said basal differentiation media is supplemented with 100 ng/mL Activin A and 25 ng/mL Wnt3A. On the beginning of day 2 (designated Day 1 in FIG. 6), and for the following two days the media is replaced with the said basal differentiation medium supplemented only with 100 ng/mL Activin A. Then on the beginning of day 4 (designated Day 3 on FIG. 6), the media is replaced with the said basal differentiation media supplemented with 30 ng/mL FGF4 and 20 ng/mL BMP2. At the beginning of Day 8 (designated Day 7 in FIG. 6), cells are rinsed twice in PBS and disaggregated with Accutase, and plated on Matrigel-coated plates in medium capable of supporting the proliferation of vascular endothelial cells supplemented with a TGFβ signaling inhibitor such as SB431542. A nonlimiting example of said endothelial media MCDB 131 supplemented with 5.0 ng/mL VEGF-A, 5.0 ng/mL FGF-2, 0.75 IU/mL heparin, 2% FBS (such as Promocell endothelial MV2 media with supplements at concentrations normally recommended by the manufacturer and sold as a complete kit (Cat# C-22022) or as cell basal medium (Cat# C-22221) and growth supplement (Cat# C-39221) and a TGFβ signaling inhibitor such as SB431542. Cells are expanded as working stocks of candidate cultures that can be expanded and cryopreserved for the purposes of deriving continuous clonal cell lines. The candidate cultures are plated at approximately 500 and 2,000 cells in 15 cm tissue culture dishes coated with Matrigel or suitable substrate for the culture of endothelial cells, and allowed to grow to visible cell colonies which are subsequently isolated by various means known in the art such as the use of cloning cylinders, and serially propagated as cell lines which are then expanded in the same media and matrix, and cryopreserved for future use.

Uses of said cells, in particular, those that have been produced in a manner such that the cells may be permanently engrafted in the host without rejection such as to produce Universal Donor Cells as described herein, including but not limited to those produced from iPS cells, that express vascular endothelial markers such as PECAM1, CDH5 (VE-Cadherin), and vWF include transplantation to increase blood flow in transplanted adipose tissue and to express ITLN1 (Omentin) or ITLN2 for therapeutic effect. Particularly useful are clonal, pooled clonal, oligoclonal, or pooled oligoclonal endothelial cell lines that express relatively high levels of ITLN1 (Omentin) or ITLN2 and are useful in imparting increased sensitivity to insulin in Type II diabetes, aged, or Syndrome X patients. Said ITLN1-expressing endothelial cell lines are may be co-injected with hydrogels, SVF and the cells of the present invention to further promote vascularization, reduce inflammatory pathways, increase insulin sensitivity in said patients. The dosage of said cells will vary from patient to patient but can be easily be determined by measuring the serum or plasma levels of Omentin in the patient. As has been reported (Zhong et al, Acta Pharmacol Sin 32: 873-878) serum omentin levels approximate 254 ng/ml+/−72.9 ng/ml in normal patients and are observed to be 113 ng/ml in patients with acute coronary syndrome, and 155 ng/ml in patients with stable angina pectoris. Plasma levels in normal patients have also been reported to be 370 ng/mL (de Souza Batista et al, Diabetes 56: 1655-1661), differences that may be attributable to differences in assay technique. Dosages will vary based on the site of injection and disease status of the patient, but will typically be 1×10⁶ to 1×10⁹ cells/patient, formulated in a suitable buffer or matrix such as hydrogels composed of crosslinked hyaluronic acid and gelatin such as HyStem-C (BioTime, Alameda, Calif.).

### Clonal Embryonic Progenitor Line Nomenclature:

Many of the human embryonic progenitor cell lines used in the work described infra have been previously described including the lines C4ELS5.1, E3, E72, E75, E163 and cells with a similar pattern of gene expression (See, e.g., US Patent Publication Nos. 20120171171 and 20100184033 both of which are incorporated by reference in their entirety). In addition, cells that express EYA4 capable of differentiating into cellular components of BAT have also been described (see WO2011/150105 entitled “Improved Methods of Screening Embryonic Progenitor Cell Lines,”) as well as (U.S. patent application Ser. No. 13/683,241, entitled “Methods of Screening Embryonic Progenitor Cell Lines”).

The clonal embryonic progenitor cell line NP110SM and cells with a similar pattern of gene expression have not been previously described. Nomenclature of the lines includes their alternative designations along with synonyms that represent minor modifications that result from the manipulation of the names resulting from bioinformatics analysis, including the substitution of “-” for “.” and vice versa, the inclusion of an “x” before cell line names beginning with an arabic number, and suffixes such as “bio1” or “bio2” that indicate biological replicates of the same line which are examples of cases where a frozen ampule of the same line was thawed, propagated, and used in a parallel analysis and “Rep1” or “Rep2” which indicate technical replicates wherein RNA isolated from a given cell line is utilized a second time for a repeat analysis without thawing or otherwise beginning with a new culture of cells. Passage number (which is the number of times the cells have been trypsinized and replated) for the cell lines is usually designated by the letter “P” followed by an arabic number, and in contrast, the population doubling number (which refers to the number of estimated doublings the cell lines have undergone in clonal expansion from one cell) is designated by the letters “PD” followed by an arabic number. The number of PDs in a passage varied from experiment to experiment but generally each trypsinization and replating was at a 1:3 to 1:4 ratio (corresponding to an increase of PDs of 1.5 and 2 respectively). In the expansion of clones, the original colonies were removed from tissue culture plates with cloning cylinders, and transferred to 24-well plates, then 12-well, and 6-well as described above. First confluent 24 well is designated P1, the first confluent 12 well culture is P2, the first 6-well culture is P3, then the six well culture was then split into a second 6 well plate (P4) and a T25 (P4). The second 6 well at P4 is utilized for RNA extraction (see U.S. Patent Publication No. 20100184033 incorporated herein by reference in its entirety) and represents about 18-21 PD of clonal expansion. Typical estimated subsequent passages and PDs are the following split to a T75 flask (19.5-22.5 PD), the P6 passage of the cells to a T225 flask (21-24 PD), then P7 being the transfer of the cells to a roller bottle (850 cm², 23-26 PD), and P8 the split into 4 rollers (25-28 PD). The ranges shown above in parenthesis represent estimated ranges in cell counts due to cell sizes, attachment efficiency, and counting error.

### Propagation of Clonal, Pooled Clonal, Oligoclonal, and Pooled Oligoclonal Cell Lines.

Aspects of the invention provide methods for identifying and differentiating embryonic progenitor cell lines that are derived from a single cell (clonal) or cell lines that are “pooled clonal” meaning that cell lines cloned have indistinguishable markers, such as gene expression markers, and are combined to produce a single cell culture often for the purpose of increasing the number of cells in a culture, or are oligoclonal wherein a line is produced from a small number, typically 2-1,000 similar cells and expanded as a cell line, or “pooled oligoclonal” lines which are lines produced by combining two or more oligoclonal cell lines that have indistinguishable markers such as patterns of gene expression. Said clonal, pooled clonal, oligoclonal, or pooled oligoclonal cell lines are then propagated in vitro through removal of the cells from the substrate to which they are affixed, and the re-plating of the cells at a reduced density of typically ⅓ to ¼ of the original number of cells, to facilitate further proliferation. Examples of said cell lines and their associated cell culture media is disclosed in U.S. patent application Ser. No. 12/504,630 filed on Jul. 16, 2009 and titled “Methods to Accelerate the Isolation of Novel Cell Strains from Pluripotent Stem Cells and Cells Obtained Thereby”; and West et al., 2008, Regenerative Medicine vol. 3(3) pp. 287-308. The compositions and methods of the present invention relate to said cell lines cultured as described but for greater than 21 doublings of clonal expansion.

### Limb Bud Mesenchyme

Unlike the human species, some invertebrate and vertebrate species show a profound capacity to regenerate any tissue damage that does not directly kill the organism. The most commonly-studied vertebrate organisms used in these studies are the Axolotls (Ambystoma mexicanum). While many tissues may be used in regeneration research in Axolotls, the most common studies involve the amputation of the limb and study of the formation of a blastema that recapitulates development in regenerating the entire functional limb. It is commonly believed that the blastema, being composed of relatively undifferentiated mesenchymal cells in a differentiated state functionally equivalent to primitive embryonic limb bud mesenchyme (ELBM) cells, is the source of these repair processes. These ELBM cells carry a pattern of site-specific homeobox gene such as HOX gene expression that facilitate the cells then forming exactly the tissues that were removed. Methods to understand this process in the human species and to apply these insights into novel methods of tissue regeneration would have widespread clinical applications to not only the tissue engineering but even regeneration in situ for applications including but not limited to limbs lost from ischemic disease, amputation, trauma, or birth defects. In the certain embodiments of the instant invention present application, we describe clonally-purified, stable, and scalable human embryonic progenitor mesenchyme cell lines isolated from hES cells, expressing lateral plate mesoderm embryonic limb progenitor markers such as HOXA10 as well as markers that discriminate between forelimb and hindlimb such as the absence of the hindlimb marker PITX1 in forelimb progenitors, that are multipotent and capable of responding to exogenously-administered extracellular factors and conditions by differentiating into the site-specific subcutaneous fat cells known as brown adipose tissue (BAT) cells. ELBM cell lines are distinct from adult or fetal-derived tissues incapable of responding to these morphogenetic signals in generating the diverse derivatives of the developing limbs. Examples of adult-derived stem cells incapable of displaying a complete regenerative phenotype are bone marrow-derived MSCs, skin-derived MSCs, adipose-derived MSCs or adipocyte stromal fraction cells, placenta and endometrium-derived MSCs, and umbilical cord-derived MSCs. Instead, the cell lines of the present invention are purified clonal, oligoclonal, pooled clonal or pooled oligoclonal lines of embryonic progenitor cells displaying a prenatal pattern of gene expression expressed in the embryonic phases of normal human development (i.e. 2-8 weeks post fertilization), such as dermal progenitors with a prenatal pattern of gene expression, displaying a capacity of scarless wound repair. Methods and uses of said cells with a prenatal pattern of gene expression including HOXA10 expression limb bud mesenchyme are described in the following: U.S. Patent Publication 20080070303, entitled “Methods to accelerate the isolation of novel cell strains from pluripotent stem cells and cells obtained thereby”; U.S. Patent Application Serial No. 20080070303 and PCT Application PCT/US2006/013519, filed on Apr. 11, 2006, entitled “NOVEL USES OF CELLS WITH PRENATAL PATTERNS OF GENE EXPRESSION”). Since regeneration of site-specific tissues such as the unique and relatively rare BAT cells associated with the interscapular regions of the upper dorsal aspect of the back of mammals, methods to manufacture large numbers of homogeneous cells in the embryonic progenitor state and with site-specific homeobox gene expression would be useful.

By way of non-limiting example, homogeneous populations of upper limb bud mesenchyme that still retains a prenatal pattern of gene expression, such as hES-derived monoclonal embryonic progenitor cell lines expressing the limb mesenchyme marker HOXA10 but lacking the expression of the lower limb marker PITX1, could be used to generate mesenchyme capable of generating brown fat progenitors or fully-differentiated BAT cells. These cells may be formulated in isotonic solutions of disaggregated cells in a relatively undifferentiated (progenitor state), or as more differentiated cells, with or without isolated adipose stromal fraction cells to provide a diversity of autologous vascular endothelial cells, perivascular cells, and WAT cell progenitors. Alternatively, the aforementioned cells may be formulated in hydrogels such as HyStem-C (BioTime, Inc. Alameda, Calif.) (BioTime, Inc. Alameda, Calif.), wherein the matrix comprises a thiol-modified gelatin and thiolated hyaluronan crosslinked in vivo or in vitro with (polyethylene glycol diacrylate (PEGDA). In other embodiments other known matrices may be used or the cells may be formulated in solution without said matrices. The cells formulated as described infra, may then be used in any of the applications described infra, including in research on stem cell biology, embryology, and tissue regeneration, or in therapy, such as to increase the number of BAT cells to reduce total body fat, increase insulin sensitivity, and decrease the risk of cardiovascular disease.

Methods of Isolating Clonal Embryonic Progenitor Cell Lines with a Pattern of Gene Expression and Differentiation Potential of the Line NP 110SM

Human pluripotent stem cells, such as human ES cells are maintained on mouse embryonic fibroblast feeder cells in hES cell culture medium consisting of DMEM with high glucose (Invitrogen, Cat#11960-044) supplemented with 20% FCS (Invitrogen, 16000), 1× non-essential amino acids (Invitrogen, Cat#12383-014), 2 mM L-Glutamine (Invitrogen, Cat#25030-081), 1% v/v Insulin Transferrin Selenium supplement (Invitrogen, Cat#41400-045), and 0.1 mM β-mercaptoethanol (Invitrogen, Cat#21985-023), either with or without 50 ng/ml FGF2 (Strathmann, 130-093-842) supplementation. To maintain and expand, hES cells are passaged either by manual micro-dissection or by enzymatic dissociation using 1 mg/ml Collagenase NB6 (Serva, 17458).

### Initial Differentiation of hES Cells for BAT Progenitor Derivation

The cells are initially differentiated in preparation for the generation of candidate cultures which function as stock cultures from which clonal progenitor cells with the gene expression profile of the NP110SM line can be isolated. In the example provide below, the hES cell line hES3 (Envy) are incubated with 1 mg/ml Collagenase for 60 minutes after which the dish was gently tapped to release the hES cell colonies into suspension. These colonies were collected and triturated to generate small clumps which were plated into ultra low attachment plates (CoStar, Corning, Cat#3471) for embryoid body (EB) formation. The EBs were formed in neural differentiation medium consisting of DMEM/F12 with Glutamax I (Invitrogen, Cat#10565-018) and 1×B27 supplement without Vitamin A (Invitrogen, Cat#12587-010) (referred to as “NP(−)” medium henceforth) and supplemented with 500 ng/ml recombinant human Noggin (R & D systems, Cat#3344-NG-050) and 20 ng/ml bFGF (Strathmann, 130-093-842). Over the next 21 days, spent medium was removed every 48 hours and fresh medium supplemented with 500 ng/ml Noggin and 20 ng/ml bFGF was added to the EBs. On day 21, spent medium was removed and fresh medium supplemented with 20 ng/ml bFGF only was added to the EBs. Reagents were sourced from Invitrogen unless otherwise stated. Neural EB formation was apparent in the culture.

### Generation of Stock Candidate Cultures

To generate candidate cultures for clonal isolation, the above-mentioned EBs on day 22 (after one day FGF2-only culture) were dissociated with Accutase (Innovative Cell Technologies, AT-104) for 10 minutes at 37° C. followed by trituration to generate a single cell suspension. The cell suspension in PBS was divided into four tubes and each aliquot was diluted with NP(−) medium (as described above)+20 ng/ml bFGF (designated NP(+) medium herein). Cells were centrifuged at 180 g for 5 minutes and each pellet was seeded into one well of a 6-well tissue culture plate in the NP(+) medium. The medium was changed 24 hours after initial plating and then 3 times a week thereafter. Upon confluence, cells in the 6-well plate were dissociated using TrypLE (Invitrogen, Cat#12563-029) for 5 minutes at 37° C. and replated in progressively larger tissue culture vessels being: T25 flask, T75 flask and T225 flask in the NP(+) medium over a period of several weeks to reach a T225 expansion stage of confluent cells. Candidate cultures of confluent cells in the T225 flask were then dissociated using TrypLE, counted and an aliquot of this single cell suspension was diluted to a concentration of 10,000 cells/ml in the NP(+) media that was used for culture to the T225 stage candidate culture stage. An aliquot of the single cell suspension was then plated at clonal dilution (500-7000 cells per 50 ml that went into the 15 cm dish) in 0.1% Gelatin-coated (Sigma, Cat# G1393) 15 cm dishes in the NP(+) medium. Remaining cells from the candidate cultures were cryopreserved (typically 3×10⁶ to 5×10⁶ cells/vial) using a controlled rate freezer program and freezing media for cryostorage and future use.

Generation of Clonal Embryonic Progenitor Cell Lines from Candidate Cultures

Cloning dishes were prepared by adding 50 ml of the above-mentioned NP(+) medium into Gelatin-coated (0.1%) 15 cm culture dishes. To each dish, a preparation of a single cell suspension from the candidate culture propagated in the NP(+) medium was then manually diluted by adding to the 15 cm culture dishes that volume of cells determined by counting a suspension of cells such that there were a selection of the following dilutions of cells; 500 cells/dish or, 1000 cells/dish or; 1500 cells/dish or; 3000 cells/dish, 5000 cells/dish or 7000 cells/dish to achieve different densities of the single cell suspension and to aid in the isolation of single colonies grown from a single cell. Alternatively, cells can be dispersed as single cells using a automatic cell deposition unit, or said automatic deposition can be used following flow sorting or other affinity purification techniques known in the art such as antibody-based selection, including monoclonal antibody-based immunoselection using flow cytometry or antibodies conjugated to magnetic beads to select cells to select cells enriched for antigens present on NP110SM cells. Such antigens may include Interleukin 13 Receptor, Alpha 2 (IL13RA2), also known as CD213A2, also known as Cancer/Testis Antigen 19.

In the case of manual dilution of cells, three separate dishes with any three of the above mentioned densities were optimised such that discrete, easily isolatable single colonies could be observed for isolation and expansion as embryonic progenitor cell lines. Seeded single cells of an appropriate dilution were distributed evenly in the dish by the sliding the 15 cm dish alternately in a clockwise, followed by counterclockwise, then side to side (left to right) motion, followed by a forward and back motion repeatedly, for about 30 seconds inside the incubator. Dishes were then incubated in a CO₂ incubator (5% CO₂, 20% O₂) and left undisturbed without moving or feeding for 14 days to allow single cells to attach to the culture dish surface and for colonies to grow to sufficient size for isolation. NP(+) media previously conditioned by the NP110SM cells for 24-48 hours can be used to increase the number and rate of proliferation of the resulting colonies.

Dishes were visually inspected and well-separated cell colonies were picked with sterile cloning cylinders (Sigma, Cat# CLS31666, CLS31668 & CLS316610) using 25 ul TrypLE for a 6 mm cylinder, 50 ul TrypLE for an 8 mm cylinder and 100 ul TrypLE for a 10 mm cylinder. Each isolated cell colony was then plated into one well each of 0.1% Gelatin-coated 24 well plates (Nunc, 142475) containing 1 ml of Promocell Smooth Muscle Cell Growth Medium 2 or its equivalent medium (designated SM medium herein). In this instance of the method, isolated embryonic progenitor cells are further cultured in the SM media. Upon confluence, cells in the 24-well plate were dissociated using TrypLE for 5 minutes at 37° C. and replated in progressively larger tissue culture vessels being: one well of a 6-well plate, T25 flask, T75 flask and T225 flask(s) in the SM medium over several weeks (an average of 1-2 weeks between each passage). Confluent cells in the T225 flask(s) were cryopreserved and banked as an isolated Embryonic Progenitor Cell Lines and seeded for immunostaining and RNA isolation, such as for PCR amplification of the transcripts for HOXA5 and IL13RA2 as a first pass screen for cells with a pattern of gene expression like that of the clonal cell line NP110SM.

Methods of Screening Embryonic Progenitor Cells for Potential for Differentiation into Cellular Components of Brown Adipose Tissue

Cells such as clonal pluripotent stem cell-derived embryonic progenitors or pooled populations of said clonal lines, or oligoclonal cultures of said progenitor cells are directly scalable cell cultures simply by serially passaging the cells in the original medium in which they were clonally expanded from a single cell and by disaggregating and replating the cells at a lower density such as a 1:2 or 1:4 split at passaging just before the cells reach confluence, thereby preventing undesired differentiation that may occur at high density. Said cells may then be exposed to differentiation conditions as described herein. By way of nonlimiting example, hES cell-derived clonal embryonic progenitor cell lines can be cultured in HyStem Bead Differentiation Condition as described herein wherein the Differentiation medium is supplemented with BMP4 at a concentration of 10 ng/mL-50 ng/mL for 14-21 days. Preferably the conditions and time of differentiation are constant with the diverse clonal embryonic progenitor cells. Differentiated cells can then be assayed for markers of differentiation by methods known in the art including gene expression reporter constructs, immunocytochemistry, and by the isolation of RNA and analysis of the mRNA transcripts in said samples by PCR or gene expression microarray. Samples that express FABP4 (accession number NM_{—}001442.1, Illumina ID 150373) and CD36 (accession number NM_{—}000072.2, Illumina ID 3310538) are considered to be differentiation into adipocytic lineages. Said cell cultures that express adipocyte markers such as FABP4 and simultaneously express the gene BETATROPHIN (accession number NM_{—}018687.3, Illumina ID 1430689) (also known as C19ORF80, LOC55908, and C19Orf80) can be considered as hits and are therefore candidates for progenitors of brown adipose tissue cells.

### Methods of Differentiating Progenitor Cells

In certain embodiments the invention provides a method of differentiating a progenitor cell in vitro, such as a hEP cell, to a more differentiated state (e.g., such as one or more of the differentiated progeny of progenitor cells described infra), relative to the starting progenitor cell, comprising contacting the progenitor cell with one or more members of the TGFβ super family. In some embodiments the TGFβ superfamily member may be chosen from TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising contacting the progenitor cell with a retinol, such as retinoic acid. The progenitor cell may be any progenitor cell disclosed infra. In one embodiment the progenitor cell is chosen from the cell lines C4ELS5.1, E3, E72, E75, E163, or NP110SM, or a cell line with the pattern of gene expression of C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising contacting the progenitor cell with a thyroid hormone such as T3 or T4. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising contacting the progenitor cell with an adrenergic agonist such as epinephrine, norepinephrine, or the highly selective beta 3-adrenergic agonist, CL316243 (J. D. Bloom, M. D. Dutia, B. D. Johnson, A. Wissner, M. G. Burns, E. E. Largis, J. A. Dolan, and T. H. Claus., J. Med. Chem. 35: 3081, 1992). The progenitor cell may be any progenitor cell disclosed infra. In one embodiment the progenitor cell is chosen from the cell lines C4ELS5.1, E3, E72, E75, E163, or NP110SM, or a cell line with the pattern of gene expression of C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising contacting the progenitor cell with physiologically-active concentrations of the growth factor FGF21. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising incubating the progenitor cell at temperatures substantially below that of normal body temperature. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In other embodiments the invention provides a method of differentiating a progenitor cell in vitro, such as a hEP cell, to a more differentiated state relative to the starting progenitor cell comprising contacting the progenitor cell with PPARγ agonists such as rosiglitazone. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In other embodiments the invention provides methods to extend the lifespan of fetal or adult-derived BAT cells or arterial smooth muscle cells such as coronary artery smooth muscle cells from individuals exposed to long-term alcohol consumption and resulting long-term exposure to relatively high levels of ketone bodies through the exogenous expression of the catalytic component of telomerase (TERT), wherein the cells can be expanded on an industrial scale and genetically modified to escape immune surveillance.

In other embodiments the invention provides a method of differentiating a progenitor cell in vitro, such as a hEP cell, to a more differentiated state relative to the starting progenitor cell comprising contacting the progenitor cell with combinations of: one or more members of the TGFβ superfamily such as TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal, a retinol, such as retinoic acid, a thyroid hormone such as T3 or T4, an adrenergic agonist such as epinephrine, norepinephrine, or the highly selective beta 3-adrenergic agonist, CL316243, and a PPARγ agonist such as rosiglitazone. The progenitor cell may be any progenitor cell disclosed infra. In one embodiment the progenitor cell is chosen from the cell lines C4ELSR2, C4ELS5.1, E3, E72, E75, E163, or NP110SM, or a cell line with the pattern of gene expression of C4ELSR2, C4ELS5.1, E3, E72, E75, E163, or NP110SM as described herein.

In one embodiment of the methods disclosed infra the progenitor cell is comprised of a micromass. In another embodiment the progenitor cell is differentiated by one or more differentiation conditions described herein and is in contact with a hydrogel. In some embodiments the progenitor cell is encapsulated within the hydrogel. The hydrogel may be comprised of hyaluronate. The hyaluronate may be thiolated. The hydrogel may be comprised of gelatin. The gelatin may be thiolated. Preferably, the hydrogel is comprised of thiolated hyaluronate or thiolated carboxymethylhyaluronate in combination with thiolated gelatin or thiolated carboxymethylgelatin. The hydrogel may comprise a crosslinker. The crosslinker may be comprised of an acrylate. In one embodiment the acrylate is PEG diacrylate.

In some embodiments the more differentiated cell expresses one or more genes described infra as being expressed by an in vitro differentiated progeny of a progenitor cell. In some embodiments the in vitro differentiated progeny express one or more genes expressed by a adipocyte, e.g. FABP4 and CD36. In some embodiments the in vitro differentiated progeny express one or more genes expressed by a BAT cell progenitor or a mature BAT cell, e.g. UCP1, ADIPOQ, or C19ORF80 (also known as BETATROPHIN). In some embodiments the differentiated progeny express one or more genes expressed by an adipose cell.

### Progeny of Progenitor Cell Lines

In certain embodiments the invention provides the progeny of a progenitor cell line. The progenitor cell line may be an embryonic progenitor cell line such as a human embryonic progenitor cell line (hEP). The progeny of the progenitor cell line may be the in vitro progeny of the progenitor cell line and may include one or more cells that are more differentiated compared to the parental progenitor cell line. The differentiation state of a cell may be determined by analyzing one or more genes expressed by the progeny cell relative to the parental progenitor cell line and/or accessing a database containing information regarding gene expression of cells at various stages of development, such as the LifeMap database. The progeny of the progenitor cell line may be a cell expressing one or more genes typically expressed by a cell in a developing mammalian embryo, such as a primate (e.g. a human). For example the progeny of the progenitor cell line may express one or more genes corresponding to an osteochondral cell fate chosen from COL2A1, COL9A2, COL10A1, MATN3, MATN4, EPYC, PTH1R, and SPP1, or cells of an adipocyte cell fate chosen from: FABP4, CD36, CIDEA, ADIPOQ, UCP1, C19orf80, NTNG1, and THRSP.

In certain embodiments the invention provides a cell culture comprising the in vitro progeny of a progenitor cell line such as a hEP cell line. In some embodiments the cell culture may comprise one or more growth factors, cytokines and/or mitogens. In certain embodiments the cell culture may comprise one or more members of the TGF-β superfamily. Exemplary members of the TGF-β superfamily include TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal. In certain embodiments the cell culture may comprise cells cultured in the presence of combinations of cytokines, factors, or conditions that induce the differentiation of brown fat cells including: culturing the cells in or without a hydrogel at temperatures substantially below normal body temperature, as described infra, with or without a differentiation agent such as a member of the TGF-β superfamily, retinoic acid, agonist of PPARγ, adrenergic agonist, and thyroid hormone. In certain embodiments the cell culture may comprise cells embedded in a hydrogel. Suitable hydrogels may comprise one or more polymers. The polymers may include any polymer known to form a hydrogel including hyaluronate, gelatin, acrylate and the like. In some embodiments the hydrogel is comprised of thiolated hyaluronate. In some embodiments the hydrogel is comprised of thiolated gelatin. In some embodiments the hydrogel is comprise of acrylate crosslinker such PEG diacrylate.

In some embodiments the invention provides a cell culture comprising the in vitro progeny of a progenitor cell line wherein the in vitro progeny of a progenitor cell line is an adipose cell precursor or a mature adipocyte. In certain embodiments of the invention the in vitro progeny of the progenitor cell line, e.g. an adipose precursor may comprise about 5% of the cells in culture, about 10% of the cells in culture, about 15% of the cells in culture, about 20% of the cells in culture, about 25% cells in culture, about 30% of the cells in culture, about 35% of the cells in culture, about 40% of the cells in culture, about 45% of the cells in culture, about 50% of the cells in culture, about 55% of the cells in culture, about 60% of the cells in culture, about 65% of the cells in culture, about 70% of the cells in culture, about 75% of the cells in culture, about 80% of the cells in culture, about 85% of the cells in culture, about 90% of the cells in culture, about 95% of the cells in culture, about 99% of the cells in culture.

### Use of HyStem-C to Cryopreserve Cells in Beads and to Modify MYH11 and FABP4 Gene Expression in Human Cells.

HyStem-C (BioTime, Inc. Alameda, Calif.) is a matrix composed of thiol-modified gelatin and thiolated hyaluronan crosslinked in vivo or in vitro with (polyethylene glycol diacrylate (PEGDA). We observed that clonal human embryonic progenitor cell lines such as those described in the present invention, could be frozen and thawed within beads of polymerized HyStem-C (BioTime, Inc. Alameda, Calif.) such as 25 μl aliquots of 2.0×10⁷ cells/mL (in FBS that is 10% DMSO) in 1% w/v HyStem-C (BioTime, Inc. Alameda, Calif.) (500,000 cells/bead). This facilitates the accumulation of large numbers of beads with large numbers of diverse hEP cell types that can be simultaneously thawed and assayed such as in high throughput robotic systems wherein the beads are exposed to diverse differentiation conditions and their differentiation assayed by gene expression microarray or other means known in the art. It also makes possible the thawing of large numbers of cryopreserved beads and the incubation of combinations of beads with diverse types of embedded cells and subsequent analysis of changes of differentiated state such as gene expression microarray or other means known in the art.

In addition, the incubation of hEP cell lines in HyStem-C (BioTime, Inc. Alameda, Calif.) allowed the accumulation of a large amount of data on the biological influence of HyStem-C (BioTime, Inc. Alameda, Calif.) on diverse cell types. With Illumina gene expression microarray data from more than 3,000 differentiation experiments, we searched for genes frequently up- and down-regulated in HyStem-C (BioTime, Inc. Alameda, Calif.) beads and compared those profiles to those obtained under micromass conditions. For example, we observed that cells cultured in HyStem-4D (Biotime, Inc. Alameda, Calif.) beads with BMP4 frequently exhibited a marked decrease in myofibroblast markers such as MYH11, and increased expression of adipocyte markers such as FABP4 and anti-inflammatory markers such as TIMP4. The cell line E15, which in other conditions was shown to have chondrogenic potential and the line W10 strongly induced MYH11 in micromass conditions supplemented with 10 ng/mL BMP4, but this induction was essentially ablated in HyStem-C (BioTime, Inc. Alameda, Calif.) culture supplemented with BMP4. Instead, in HyStem-C (BioTime, Inc. Alameda, Calif.) beads, the line markedly upregulated expression of DCN, a marker of meninges. This physiological effect on myofibroblastic differentiation seen in many lines cultured in HyStem-C (BioTime, Inc. Alameda, Calif.) beads (i.e., the strong reduction in MYH11 expression) has therapeutic implications in vivo, such as in inhibiting fibrosis or adhesions. It also is of benefit in surgical settings where cells could be transplanted to regenerate tissue function while inhibiting adhesions and related fibrotic process at the surgical site.

As previously described (see, U.S. patent application Ser. No. 14/048,910, incorporated by reference) diverse clonal embryonic progenitor cell lines show correspondingly diverse differentiation responses to growth factors such as members of the TGF-beta superfamily. In some cases, including but not limited to the culture of the cells in HyStem-C (BioTime, Inc. Alameda, Calif.) beads in the presence of BMP4, some cell lines strongly express markers of adipocytes such as FABP4 and CD36. Because the clonal progenitor cell lines capable of adipocyte differentiation represent mesenchymal anlagen of diverse anatomical origin, the corresponding adipocytes may represent fat-forming cells with diverse phenotypes. Some of these diverse phenotypes offer novel therapeutic opportunities as described herein.

We disclosed that one subset of therapeutically-useful adipocytes are those expressing the adipokine C19orf80 (also known as betatrophin or ANGPTL8, encoded by the human gene C19orf80, accession number NM_{—}018687.3). These cells express upper limb markers such as HOXA10 and HOXD11, but lack distal HOX genes such as HOXC9, HOXC10, or HOXC11. The cell lines of the present invention that display this pattern of gene expression include E72, E75 and E163. The cell line E72 expresses HOXA10 (accession number NM_{—}153715.2, Illumina ID 3290427), POSTN (accession number NM_{—}006475.1, Illumina ID 510246), KRT34 (accession number NM_{—}021013.3, Illumina ID 3710168), MKX (accession number NM_{—}173576.1, Illumina ID 6620017), HAND2 (accession number NM_{—}021973.2, Illumina probe ID 4640563), the relatively rarely-expressed HOX gene HOXD11 (accession number NM_{—}021192.2, Illumina probe ID 5290142) implicated in forelimb development, and TBX15 (accession number NM_{—}152380.2, Illumina probe ID 6060113), but does not express LHX8 (accession number NM_{—}001001933.1, Illumina ID 2900343), FOXF2 (accession number NM_{—}001452.1, Illumina ID 1660470), AJAP1 (accession number NM_{—}018836.3, Illumina ID 1300647), PLXDC2 (accession number NM_{—}032812.7, Illumina ID 5900497), or DLK1 (accession number NM_{—}003836.4, Illumina ID 6510259).

The line E72 did not express relatively distal HOX genes such as HOXB7 (accession number NM_{—}004502.2, Illumina probe ID 2470328), and HOXC8 (accession number NM_{—}022658.3, Illumina probe ID 4640059) expressed by cultured MSCs from the iliac crest, or the HOX genes HOXC9, HOXC10, or HOXC11 expressed in hindlimb, but not forelimb bud mesenchyme. The cell lines E75 and E163 expressed the same markers as E72, but unlike the line E72 which did not express PLXDC2 (accession number NM_{—}032812.7, Illumina probe ID 5900497), the lines E75 and E163 did express PLXDC2.

The cell lines E72, E75, and E163, or cells with a similar pattern of gene expression, are capable of differentiating into C19orf80-expressing adipocytes when exposed to adipogenic differentiation conditions such as differentiation in HyStem-C (BioTime, Inc. Alameda, Calif.) as described with chondrogenic medium supplemented with 10 ng/mL BMP4 for 14-21 days, but without TGFβ3. Bone marrow-derived mesenchymal stem cells (MSCs), differentiated in HyStem-C (BioTime, Inc. Alameda, Calif.) beads in the presence of 50 ng/mL of BMP2, or 10 ng/mL of BMP4, or 100 ng/mL of BMP7 for 14 days caused the differentiation of the cells into adipocytes as evidenced by their expression of FABP4, whereas in the undifferentiated state, the MSCs did not express detectable FABP4. Similarly, the lines E72, E75, E163, as well as clonal hES-derived embryonic progenitors corresponding to other anatomic locations, also differentiated into FABP4-expressing cells. However, only MSCs, E72, E75, and E163 cells induced C19orf80/betatrophin expression upon differentiation. Interestingly, the lines described capable of differentiating into C19orf80-expressing adipocytes also induced the expression of HEPACAM upon differentiation, a marker potentially useful in assays of purity or useful in purifying said cells by methods such as affinity purification.

The HEPACAM+, C19orf80(C19orf80)+ adipocytes of the present invention are useful in producing the secreted protein C19orf80 which in turn is useful in inducing the proliferation of pancreatic beta cells either in vitro or in vivo. Said beta cells may be beta cells cultured in vitro wherein said cells are derived from mammalian pancreas or derived from cultured pluripotent stem cells such as hES or human iPS cells.

The C19orf80-expressing adipocytes including said adipocytes derived by differentiating bone marrow-derived MSCs, or the hES-derived clonal embryonic progenitor cell lines E72, E75, or E163, or adipocytes differentiated from pluripotent stem cell-derived cells with the above-described pattern of gene expression, are also useful in treating type I and type II diabetes. In said therapeutic applications, the C19orf80-expressing adipocytes of the present invention may be injected into the body, by way of nonlimiting example, the cells in a concentration of 2.5×10⁵ cells/ml to 1.0×10⁸ cells/ml in HyStem-C (BioTime, Inc. Alameda, Calif.), preferably 1.0×10⁷ cells/ml, or at these concentrations in other matrices useful in promoting cell engraftment. The site of engraftment may vary, but by way of example, the cells may be injected subcutaneously at the normal site of brown fat cells in humans such as in the interscapular region of the back. The cells may or may not also be genetically-modified, with modifications to increase C19orf80 expression, such as those that down-regulate the insulin receptor gene, or allow the inducible apoptosis of the engrafted cells, or modification to promote the allogeneic histocompatibility of said cells.

In some applications, said C19orf80-expressing adipocytes are mitotically inactivated as described herein to limit their lifespan and lead to a transient expression of C19orf80 to transiently induce the proliferation of pancreatic beta cells.

In aged patients, pancreatic beta cell proliferation in response to the transplanted C19orf80-secreting adipocytes or alternatively, the pancreatic beta cell proliferation in response to administered C19orf80 protein, or simply the pancreatic beta cell proliferation in response hypoinsulinemia, can be facilitated by the extension of telomere length in the beta cells or beta cell precursors by the exogenous expression of the catalytic component of telomerase reverse transcriptase, such as human TERT. The telomerase catalytic component of telomerase may be introduced by varied methods known in the art such as viral gene therapy, including but not limited to adenoviral vectors.

Provided herein are improved methods and compositions comprising hEP cells and their differentiated progeny as well as methods for directing the differentiation of hEP cells to mature brown fat cells useful in research and for treating certain metabolic and vascular disorders.

### Methods and Compositions for Cryo-Preserving Cells

In some embodiments the invention provides methods for cryo-preserving the cells described infra. In other embodiments the invention provides compositions comprising cryopreserved cells, wherein the cell is one or more cells described infra.

In certain embodiments the invention provides a composition comprising a cryopreserved progenitor cell, such as hEP cell described infra. The composition may comprise at least 1 progenitor cell, at least 10, at least 100, at least 1,000, at least 10,000, at least 100,00, at least 1,000,000 viable cryo preserved progenitor cells. The composition may comprise about 1 progenitor cell, about 10, about 100, about 1,000, about 10,000, about 100,000, about 1,000,000 viable cryo preserved progenitor cells. The cryopreserved progenitor cell may further comprise a hydrogel wherein the progenitor cell is seeded within the hydrogel. The cryopreserved progenitor cell may include a suitable media containing one or more cryoprotectants, such as DMSO or FBS to facilitate freezing the cells. In one embodiment the invention provides a hEP cell cryopreserved in a hydrogel comprising hyaluronate. The hydrogel may further comprise gelatin. The hydrogel may further comprise an acrylate such as PEG acrylate. The acrylate may serve as a crosslinker. An example of a suitable media for cryo-preserving the cells in a hydrogel may comprise FBS that is 10% DMSO. The cells may frozen at −80° C.

In other embodiments the invention provides a composition comprising a cryo-preserved in vitro differentiated progeny of a progenitor cell, such as hEP cell described infra. The composition may comprise at least 1, at least 10, at least 100, at least 1,000, at least 10,000, at least 100,00, at least 1,000,000 viable cryo preserved in vitro differentiated progeny of a progenitor cell. The composition may comprise about 1, about 100, about 1,000, about 10,000, about 100,00, about 1,000,000 viable cryo preserved in vitro differentiated progeny of a progenitor cell. The cryopreserved in vitro differentiated progeny of a progenitor cell may further comprise a hydrogel wherein the in vitro differentiated progeny of a progenitor cell is seeded within the hydrogel. The cryopreserved in vitro differentiated progeny of a progenitor cell may include a suitable media containing one or more cryoprotectants, such as DMSO or FBS to facilitate freezing the cells. In one embodiment the invention provides the in vitro differentiated progeny of an hEP cell cryopreserved in a hydrogel comprising hyaluronate. The hydrogel may further comprise gelatin. The hydrogel may further comprise an acrylate such as PEG acrylate. The acrylate may serve as a crosslinker. An example of a suitable media for cryo-preserving the cells in a hydrogel may comprise FBS that is 10% DMSO. The cells may frozen at −80° C.

The cryopreserved compositions may be used in research and therapeutic applications. For example a subject in need of cell therapy may be treated with the cryopreserved composition described infra. The composition may be thawed and administer to a subject in need of treatment. The placement of the cells described infra in the hydrogel may facilitate both cropreserving the cell and enhancing transplantation of the cell into a subject.

In some embodiments the invention provides a method of cryo-preserving a cell comprising 1) contacting the cell with a hydrogel, 2) contacting the cell of 1) with a media comprising fetal bovine serum (FBS) and dimethyl sulfoxide (DMSO) and 3) freezing the cell of 2) at −80° C. thereby cryo-preserving the cell.

In some embodiments the invention provides a method of cryo-preserving a cell comprising 1) contacting the cell with a hydrogel, 2) contacting the cell of 1) with a media comprising fetal bovine serum (FBS) and glycerol and 3) freezing the cell of 2) at −80° C. thereby cryo-preserving the cell.

In some embodiments the method described in the previous paragraph is practiced using one or more of the cells described infra. Thus the cell may be a hEP cell or the in vitro differentiated progeny of a hEP cell. The cell may be contacted with the hydrogel before the hydrogel has had a chance to solidify, e.g. the may be contacted with one or more liquid preparations comprising the hydrogel and after contacting the cell with the one or more liquid preparations comprising the hydrogel the hydrogel may be allowed to polymerize. The hydrogel may comprise hyaluronate, gelatin and a crosslinker such as an acrylate or methacrylate, e.g., PEG acrylate. The hyaluronate may be thiolated. The gelatin may be thiolated. (See U.S. Pat. Nos. 7,928,069; 7,981,871). The hydrogel may be seeded with about 100 cells, about 500 cells, about 1,000 cells, about 10,000 cells, about 100,000 cells, about 1,000,000 cells, about 10,000,000 cells. In some embodiments the hydrogel is seeded with about 10⁵-to about 10⁷ cells.

The media used in the method of cryo-preserving cells described infra may comprise any known media and a suitable cryoprotectant. Examples of suitable cryoprotectants include FBS, DMSO, glycerol, glucose and the like. In one embodiment the media is comprised of FBS that is made 10% DMSO. In another embodiment the media consists of FBS that is made 10% DMSO.

### General Techniques

Laboratory techniques useful in the practice of this invention can be found in standard textbooks and reviews in cell biology, tissue culture, and embryology. Stem cell biology and manipulation is described in Teratocarcinomas and embryonic stem cells: A practical approach, by E. J. Robertson ed., IRL Press Ltd. 1987; Guide to Techniques in Mouse Development, by P. M. Wasserman et al. eds., Academic Press 1993; and Embryonic Stem Cell Differentiation in Vitro, by M. V. Wiles, Meth. Enzymol. 225:900 1993.

Methods in molecular genetics and genetic engineering are described in Molecular Cloning: A Laboratory Manual, 2.sup.nd Ed., by Sambrook et al. 1989; Oligonucleotide Synthesis, by M. J. Gait ed. 1984; Animal Cell Culture, by R. I. Freshney ed. 1987; the series Methods in Enzymology, by Academic Press; Gene Transfer Vectors for Mammalian Cells, by J. M. Miller & M. P. Calos eds. 1987; Current Protocols in Molecular Biology and Short Protocols in Molecular Biology, 3.sup.rd Edition, by F. M. Ausubel et al. eds. 1987 & 1995; and Recombinant DNA Methodology II, by R. Wu ed., Academic Press 1995. Reagents, cloning vectors, and kits for genetic manipulation referred to in this disclosure are available from commercial vendors such as BioRad, Stratagene, Invitrogen, and ClonTech. General techniques used in raising antibodies, and the design and execution of immunoassays and immunohistochemistry, are found in the Handbook of Experimental Immunology, by D. M. Weir & C. C. Blackwell eds.; Current Protocols in Immunology, by J. E. Coligan et al. eds. 1991; and R. Masseyeff, W. H. Albert, and N. A. Staines eds., Methods of Immunological Analysis, by Weinheim: VCH Verlags GmbH 1993.

### Applications

The disclosed methods for the culture of animal cells and tissues are useful in generating brown fat progenitors and differentiated brown fat cells for use in research and therapy. Research uses include the use of the cells in drug screening for agents useful in treating metabolic disorders and therapeutic uses include the use of the cells or progeny thereof in mammalian and human cell therapy, such as, but not limited to, generating human cells useful in treating metabolic and vascular disorders in humans and nonhuman animals.

The methods used in the present invention wherein the original pluripotent stem cells are used as master cell banks for the indefinite derivation on an industrial scale of differentiated cell types has commercial advantages in quality control and reproducibility. Of particular utility is the present invention wherein the master cell bank may be genetically modified to allow the resulting somatic cells to escape immune surveillance, and where an intermediate still relatively undifferentiated clonal embryonic progenitor cell type with relatively long telomere length is scaled up as the point of industrial scalability. Also of particular utility are the formulations described herein where the cells of the present invention are differentiated with factors that induce BAT cell differentiation in a hydrogel that has been demonstrated to be safely administered subcutaneously in humans, thereby providing a formulation to produce three-dimensional adipose tissue in vivo.

### Drug Screening

The cells of this invention can be used to screen for factors (such as solvents, small molecule drugs, peptides, polynucleotides) or environmental conditions (such as culture conditions or manipulation) that affect the characteristics of both brown fat preadipocyte precursors and mature brown fat cells.

In one example, pluripotent stem cells (undifferentiated or initiated into the differentiation paradigm) are used to screen factors that promote maturation into brown fat cells, or promote proliferation and maintenance of brown fat cells in long-term culture. For example, candidate maturation factors or growth factors are tested by adding them to cells in different wells, and then determining any phenotypic change that results, according to desirable criteria for further culture and use of the cells. This can lead to improved derivation and culture methods not only for pluripotent stem cell-derived brown fat cells, but for brown fat cell progenitors isolated from fetal or adult tissue.

Another example is the use of brown fat cell progenitors or differentiated brown fat cells of the present invention are used to measure the effect of small molecule drugs that have the potential to affect brown fat cell activity in their role of metabolizing lipoproteins, secreting adipokines, or heat regulation. To this end, the cells can be combined with test compounds in vitro, and the effect of the compound on gene expression or protein synthesis can be determined. The screening can also be done in vivo by measuring the effect of the compound on the behavior of the cells in an animal model.

Other screening methods of this invention relate to the testing of pharmaceutical compounds for a potential effect on brown fat cell growth, development, or toxicity. This type of screening is appropriate not only when the compound is designed to have a pharmacological effect on brown fat cells themselves, but also to test for brown fat cell-related side-effects of compounds designed for a primary pharmacological effect elsewhere.

The reader is referred generally to the standard textbook “In vitro Methods in Pharmaceutical Research”, Academic Press, 1997, and U.S. Pat. No. 5,030,015. Assessment of the activity of candidate pharmaceutical compounds generally involves combining the differentiated cells of this invention with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change.

Cytotoxicity can be determined in the first instance by the effect on cell viability, survival, morphology, and the expression of certain markers and receptors. Effects of a drug on chromosomal DNA can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to A. Vickers (pp 375-410 in “In vitro Methods in Pharmaceutical Research,” Academic Press, 1997) for further elaboration.

In certain embodiments of the invention, the differentiated progeny of hEP cells described infra, may be used as “feeder cells” to support the growth of other cell types, including pluripotent stem cells. The use of the differentiated progeny of hEP cells of the present invention as feeder cells alleviates the potential risk of transmitting pathogens from feeder cells derived from other mammalian sources to the target cells. The feeder cells may be inactivated, for example, by gamma ray irradiation or by treatment with mitomycin C, to limit replication and then co-cultured with the pluripotent stem cells.

In certain embodiments of the invention, the extracellular matrix (ECM) of the differentiated progeny of hEP cell disclosed infra, may be used to support less differentiated cells (see Stojkovic et al., Stem Cells (2005) 23(3):306-14). Certain cell types that normally require a feeder layer can be supported in feeder-free culture on a matrix (Rosler et al., Dev Dyn. (2004) 229(2):259-74). The matrix can be deposited by pre-culturing and lysing a matrix-forming cell line (see WO 99/20741), such as the STO mouse fibroblast line (ATCC Accession No. CRL-1503), or human placental fibroblasts.

In certain embodiments of the invention, the conditioned media of differentiated progeny of hEP cells may be collected, pooled, filtered and stored as conditioned medium. This conditioned medium may be formulated and used for research and therapy. The use of conditioned medium of cell cultures described infra may be advantageous in reducing the potential risk of exposing cultured cells to non-human animal pathogens derived from other mammalian sources (i.e. xenogeneic free).

In another embodiment of the invention, single cell-derived and oligoclonal cell-derived cells and their differentiated progeny described infra may be used as a means to identify and characterize genes that are transcriptionally activated or repressed as the cells undergo differentiation. For example, libraries of gene trap single cell-derived or oligoclonal cell-derived cells and/or their differentiated progeny may be made by methods of this invention, and assayed to detect changes in the level of expression of the gene trap markers as the cells differentiate in vitro and in vivo. The methods for making gene trap cells and for detecting changes in the expression of the gene trap markers as the cells differentiate are reviewed in Durick et al. (Genome Res. (1999) 9:1019-25). The vectors and methods useful for making gene trap cells and for detecting changes in the expression of the gene trap markers as the cells differentiate are also described in U.S. Pat. No. 5,922,601 (Baetscher et al.), U.S. Pat. No. 6,248,934 (Tessier-Lavigne) and in U.S. patent publication No. 2004/0219563 (West et al.). Methods for genetically modifying cells, inducing their differentiation in vitro, and using them to generate chimeric or nuclear-transfer cloned embryos and cloned mice are developed and known in the art. To facilitate the identification of genes and the characterization of their physiological activities, large libraries of gene trap cells having gene trap DNA markers randomly inserted in their genomes may be prepared. Efficient methods have been developed to screen and detect changes in the level of expression of the gene trap markers as the cells differentiate in vitro or in vivo. In vivo methods for inducing single cell-derived or oligoclonal cell-derived cells or their differentiated progeny to differentiate further include injecting one or more cells into a blastocyst to form a chimeric embryo that is allowed to develop; fusing a stem cell with an enucleated oocyte to form a nuclear transfer unit (NTU), and culturing the NTU under conditions that result in generation of an embryo that is allowed to develop; and implanting one or more clonogenic differentiated cells into an immune-compromised or a histocompatible host animal (e.g., a SCID mouse, or a syngeneic nuclear donor) and allowing teratomas comprising differentiated cells to form. In vitro methods for inducing single cell-derived or oligoclonal cell-derived cells to differentiate further include culturing the cells in a monolayer, in suspension, or in three-dimensional matrices, alone or in co-culture with cells of a different type, and exposing them to one of many combinations of chemical, biological, and physical agents, including co-culture with one or more different types of cells, that are known to capable of induce or allow differentiation.

In another embodiment of the invention, cell types that do not proliferate well under any known cell culture conditions may be induced to proliferate such that they can be isolated clonally or oligoclonally according to the methods of this invention through the regulated expression of factors that overcome inhibition of the cell cycle, such as regulated expression of SV40 virus large T-antigen (Tag), or regulated E1a and/or E1b, or papillomavirus E6 and/or E7, or CDK4 (see, e.g., U.S. patent application Ser. No. 11/604,047 filed on Nov. 21, 2006 and titled “Methods to Accelerate the Isolation of Novel Cell Strains from Pluripotent Stem Cells and Cells Obtained Thereby”, incorporated herein by reference).

In another embodiment of the invention, the factors that override cell cycle arrest may be fused with additional proteins or protein domains and delivered to the cells. For example, factors that override cell cycle arrest may be joined to a protein transduction domain (PTD). Protein transduction domains, covalently or non-covalently linked to factors that override cell cycle arrest, allow the translocation of said factors across the cell membranes so the protein may ultimately reach the nuclear compartments of the cells. PTDs that may be fused with factors that override cell cycle arrest include the PTD of the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, the amino acid sequence conferring membrane translocation activity corresponds to residues 47-57 (Ho et al., 2001, Cancer Research 61: 473-477; Vives et al., 1997, J. Biol. Chem. 272: 16010-16017). These residues alone can confer protein translocation activity.

### BAT Cell Progenitors and BAT Cells in Clinical Therapy

This invention also provides for the use of BAT precursor and fully differentiated BAT cells and their derivatives to retain or restore normal metabolism in a patient in need of such therapy. Any condition leading to impairment of fat, lipoprotein, blood pressure, or glucose metabolism may be considered. Included are conditions commonly associated with metabolic syndrome X. The cells of the invention can also be considered for treatment of Type I diabetes, wherein betatrophin-secreting cells are injected into the pancreas. Also contemplated is the use of the cells of this invention for the management of obesity and coronary disease.

In certain embodiments of the invention, single cell-derived and oligoclonal cell-derived cells and their differentiated progeny as described infra are utilized in the treatment of disorders relating to cell biology, adipocyte differentiation, and lipoprotein metabolism. For example the hEP cells and their differentiated progeny may be used to generate cDNA libraries which in turn could be used to study gene expression in developing tissue, such as fat, including brown fat cells expressing critical adipokines such as betatrophin or adiponectin and for studying the inherited expression levels of IL13RA2 as a risk factor for obesity and Type II diabetes. The hEP cells and their differentiated progeny can be used in drug screening. For example the cell, such as a differentiated progeny of hEP cell could be contacted with a test drug or compound and analyzed for toxicity by examining the cells under a microscope and observing their morphology or by studying their growth or survival in culture. The cells may also be screened for gene expression to determine the effects of the drug, in particular, for inducing the browning of fat by assaying for UCP1, ADIPOQ, or C19ORF80 expression. For example, a comparison could be made between a differentiated progeny of hEP cell that has been contacted with the test drug or compound compared with the same differentiated progeny cell that has not been so contacted.

The differentiated progeny of hEP cells may be used to screen for the effects of growth factors, hormones, cytokines, mitogens and the like to determine the effects of these test compounds on the differentiation status of the differentiated progeny of the hEP cells.

In certain embodiments of the invention, the differentiated progeny of the hEP cells may be introduced into the tissues in which they normally reside in order to exhibit therapeutic utility or alternatively to coax the cells to differentiate further. In certain embodiments of the invention, the differentiated progeny of the hEP cells described infra, are utilized in inducing the differentiation of other pluripotent or multipotent stem cells. Cell-cell induction is a common means of directing differentiation in the early embryo. Cell types useful in the induction may mimic induction well known in the art to occur naturally in normal embryonic development.

Many potentially medically-useful cell types are influenced by inductive signals during normal embryonic development, including spinal cord neurons, cardiac cells, pancreatic beta cells, and definitive hematopoietic cells. Differentiated progeny of hEP cells may be cultured in a variety of in vitro, or in vivo culture conditions to induce the differentiation of other pluripotent stem cells to become desired cell or tissue types. Induction may be carried out in a variety of methods that juxtapose the inducer cell with the target cell. By way of nonlimiting examples, the inducer cells may be plated in tissue culture and treated with mitomycin C or radiation to prevent the cells from replicating further. The target cells are then plated on top of the mitotically-inactivated inducer cells. Alternatively, the differentiated progeny of hEP cells may be cultured on a removable membrane from a larger culture of cells or from an original single cell-derived colony and the target cells may be plated on top of the inducer cells or a separate membrane covered with target cells may be juxtaposed so as to sandwich the two cell layers in direct contact. The resulting bilayer of cells may be cultured in vitro, transplanted into a SPF avian egg, or cultured in conditions to allow growth in three dimensions while being provided vascular support (see, for example, international patent publication number WO/2005/068610, published Jul. 28, 2005, the disclosure of which is hereby incorporated by reference). The inducer cells may also be from a source of differentiated progeny of hEP cells, in which a suicide construct has been introduced such that the inducer cells can be removed at will.

The cells of the present invention are optimally formulated for therapeutic use when combined with a biocompatible matrix such as HyStem-C (Renevia). BAT cells are prepared by growing in cell culture on tissue culture vessel surfaces or beads in a slurry, or alternatively on HyStem-C beads wherein they are frozen for use at the point of care. During surgery, the BAT cells are thawed, mixed with matrix components, and the cell-loaded matrix is injected into the area of desired placement.

BAT cells made pursuant to the instant invention may also be formulated with patient-specific adipose stromal vascular fraction such as that obtained in abdominal liposuction to provide cellular components of normal adipose tissue vasculature including vascular endothelial, and perivascular cells to aid in the vascularization and survival of the graft. In addition, the graft may be augmented with pluripotent stem cell-derived vascular progenitors expressing ITLN1 and ITLN2 such as that previously described (Compositions and methods relating to clonal progenitor cell lines, WO 2013036969 A1). As always, the ultimate responsibility for patient selection, mode of administration, and choice of support structures and surgical options is the responsibility of the managing clinician.

For purposes of commercial distribution, BAT cells of this invention are typically supplied in the form of a pharmaceutical composition, comprising an isotonic excipient prepared under sufficiently sterile conditions for human administration. For general principles in medicinal formulation of cell compositions, the reader is referred to Cell Therapy: Stem Cell Transplantation, Gene Therapy, and Cellular Immunotherapy, by G. Morstyn & W. Sheridan eds, Cambridge University Press, 1996.

The composition may also contain a matrix for keeping the BAT cells in place during the first few months following therapy. Biocompatible matrices such as HyStem (BioTime) allow the mixture of cells with matrix, the injection of said cells and matrix in a liquid form, with polymerization forming in vivo. Besides HyStem, other possible matrixes include bioresorbable polymer fleece matrices (Rudert et al., Cells Tissues Organs 167:95, 2000); hyaluronan derivatives (Grigolo et al., Biomaterials 22:2417, 2001); sponge made from poly(L-lactide-epsilon-caprolactone) (Honda et al., J. Oral Maxillofac. Surg. 58:767, 2000), and collagen-fibrin matrices (Clin. Exp. Rheumatol. 18:13, 2000).

The cells of the present invention may be transplanted to increase insulin sensitivity, to decrease total body fat, or to decrease coronary or stroke disease risk by transplanting the cells at a dosage, by way of nonlimiting example, in humans, cells may be administered in the intercostal region where BAT cells normally reside at birth and in the vicinity of sympathetic innvervation of such BAT cells at a concentration of 2.5×10⁵ cells/ml to 1.0×10⁸ cells/ml in HyStem-C (BioTime, Inc. Alameda, Calif.), preferably 1.0-3.0×10⁷ cells/ml, or at these concentrations in other matrices useful in promoting cell engraftment. The total dosage of said cells administered will vary based on the extent of the loss of BAT tissue and the severity of the disease. For example, patients with morbid obesity may require cells administered at the upper ranges described herein based on the judgement of the patient's physician. Individual doses will vary from 10-100×10⁶ cells per injection (0.3 mL-10.0 ml per injection depending on concentration of cells). Effectiveness of the therapy can be assessed by monitoring serum adiponectin and/or betatrophin by ELISA or other means known in the art before and after treatment, or by PET scanning following administration in vivo of 2-[¹⁸F]fluoro-2-deoxyglucose (FDG) to assess uptake into BAT tissue.

The site of engraftment may vary, but by way of example, the cells may be injected subcutaneously at the normal site of brown fat cells in humans such as in the interscapular region of the back. The cells may or may not also be genetically-modified, with modifications to increase C19orf80 expression, such as those that down-regulate the insulin receptor gene, or allow the inducible apoptosis of the engrafted cells, or modification to promote the allogeneic histocompatibility of said cells.

In the prevention of atherosclerosis, such as coronary artery disease, the BAT cells of the present invention, formulated in HyStem-C at comparable concentrations disclosed herein, may be injected into the perivascular space surrounding arteries at risk for or displaying atherosclerosis. The presence of the BAT cells of the present invention will provide a therapeutic effect to the patient through the unique lipoprotein metabolism displayed by the cells as well as the secretion of adiponectin.

In the management of Type I and Type II diabetes, cells of the present invention, including without limitation, human ES cell-derived clonal embryonic progenitor cell lines expressing a pattern of genes conferring immunotolerance as described herein and a pattern of gene expression comparable to NP110SM and expressing relatively high levels of C19ORF80 in the differentiated state may be injected directly into the pancreas to induce beta cell proliferation. When said cells also overexpress localized immunosuppressive agents such as PD-L1, such cells can also be used to halt the immune-mediated destruction of pancreatic beta cells in Type I diabetes.

The composition or device is optionally packaged in a suitable container with written instructions for a desired purpose, such as the reconstruction of BAT tissue for the management of obesity, diabetes, and coronary disease.

It is understood that certain adaptations of the invention described in this disclosure as a matter of routine optimization for those skilled in the art, and can be implemented without departing from the spirit of the invention, or the scope of the appended claims.

In another embodiment of the invention, the hEP cell line such as an hEP cell line capable of brown fat differentiation may be immortalized or have its cell lifespan extended by the permanent or temporary expression of the catalytic component of telomerase (TERT).

In another embodiment of the invention, the single cell-derived and oligoclonal cell-derived cells or their differentiated progeny may be used to generate ligands using phage display technology (see U.S. application No. 60/685,758, filed May 27, 2005, and PCT US2006/020552). The expression of genes of the cells of this invention may be determined Measurement of the gene expression levels may be performed by any known methods in the art, including but not limited to, microarray gene expression analysis, bead array gene expression analysis and Northern analysis. The gene expression levels may be represented as relative expression normalized to the ADPRT (Accession number NM_{—}001618.2), GAPDH (Accession number NM_{—}002046.2), or other housekeeping genes known in the art. The gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines. Based on the gene expression levels, one would expect the expression of the corresponding proteins by the cells of the invention.

In another embodiment of the invention, the single cell-derived or oligoclonal cell-derived cells or their differentiated progeny described infra may express unique patterns of CD antigen gene expression, which are cell surface antigens. The differential expression of CD antigens on the cell surface may be useful as a tool, for example, for sorting cells using commerically available antibodies, based upon which CD antigens are expressed by the cells. The expression profiles of CD antigens of some cells of this invention are shown in West et al., 2008, Regen Med vol. 3(3) pp. 287-308, incorporated herein by reference, including supplemental information. There are several CD antigens that are expressed in the relative more differentiated cells of this invention, but are not expressed in ES cells (or in some cases at markedly reduced levels). The antigens that fall into this category include: CD73, CD97, CD140B, CD151, CD172A, CD230, CD280, CDw210b. These antigens may be useful in a negative selection strategy to grow ES cells or alternatively to isolate certain cells described infra.

In another embodiment of the invention, the single cell-derived and oligoclonal cell-derived cells or their differentiated progeny, may be injected into mice to raise antibodies to differentiation antigens. Antibodies to differentiation antigens would be useful for both identifying the cells to document the purity of populations for cell therapies, for research in cell differentiation, as well as for documenting the presence and fate of the cells following transplantation. In general, the techniques for raising antibodies are well known in the art.

In another embodiment of the invention, the single cell-derived and oligoclonal cell-derived cells or the differentiated progeny thereof may be used for the purpose of generating increased quantities of diverse cell types with less pluripotentiality than the original stem cell type, but not yet fully differentiated cells. mRNA or miRNA can then be prepared from these cell lines and microarrays of their relative gene expression can be performed as described herein.

In another embodiment of the invention, the single cell-derived and oligoclonal cell-derived cells or their differentiated progeny may be used in animal transplant models, e.g. transplanting escalating doses of the cells with or without other molecules, such as ECM components, to determine whether the cells proliferate after transplantation, where they migrate to, and their long-term differentiated fate in safety studies.

In another embodiment of the invention, the cells of the present invention when induced to differentiate into BAT cell components expressing betatrophin and adiponectin into the medium may be used as a means of manufacturing said proteins for research and therapeutic use using the spent media or using methods described herein for the mild urea extraction of secreted proteins or simply collecting spent media and purifying the proteins to varying levels of purity.

In another embodiment of the invention, the single cell-derived and oligoclonal cell-derived cells generated according to the methods of the present invention are useful for harvesting mRNA, microRNA, and cDNA from either single cells or a small number of cells (i.e., clones) to generate a database of gene expression information. This database allows researchers to identify the identity of cell types by searching for which cell types in the database express or do not express genes at comparable levels of the cell type or cell types under investigation. For example, the relative expression of mRNA may be determined using microarray analysis as is well known in the art. The relative values may be imported into a software such as Microsoft Excel and gene expression values from the different cell lines normalized using various techniques well known in the art such as mean, mode, median, and quantile normalization. Hierarchical clustering with the single linkage method may be performed with the software such as The R Project for Statistical Computing as is well known in the art. An example of such documentation may be found online. A hierarchical clustering analysis can then be performed as is well known in the art. These software programs perform a hierarchical cluster analysis using a group of dissimilarities for the number of objects being clustered. At first, each object is put in its own cluster, then iteratively, each similar cluster is joined until there is one cluster. Distances between clusters are computed by Lance-Williams dissimilarity update formula (Becker, R. A., Chambers, J. M. and Wilks, A. R. (1988) The New S Language. Wadsworth & Brooks/Cole. (S version.); Everitt, B. (1974). Cluster Analysis. London: Heinemann Educ. Books). Typically the vertical axis of the dendograms displays the extent of similarity of the gene expression profiles of the cell clones. That is, the farther down they branch apart, the more similar they are. The verticle axis is a set of n−1 non-decreasing real values. The clustering height is the value of the criterion associated with the clustering method for the particular agglomeration. In order to determine if a new cell line is identical to existing cell lines, two types of replicates are performed: biological and technical replicates. Biological replicates require that new cell lines be grown, mRNA harvested, and then the analysis compared. Technical replicates, on the other hand, analyze the same RNA twice. A line cutoff is then drawn just above where the replicates branch such that cells branching below the cutoff line are considered the same cell type. Another source of data for the database described above may be microRNA profiles of the single cell-derived and oligoclonal cell-derived cells or their differentiated progeny described infra. MicroRNAs (miRNA) are endogenous RNAs of ˜22 nucleotides that play important regulatory roles in animals & plants by targeting mRNAs for cleavage or translational repression. More than 700 miRNAs have been identified across species. Their expression levels vary among species and tissues. Low abundant miRNAs have been difficult to detect based on current technologies such as cloning, Northern hybridization, and the modified Invader® assay. In the present invention, an alternative approach using a new real-time quantitation method termed looped-primer RT-PCR was used for accurate and sensitive detection of miRNAs as well as other non-coding RNA (ncRNA) molecules present in human embryonic stem cells and in cell lines differentiated from human embryonic stem cells.

In another embodiment of the invention, gene expression analysis may be used to identify the developmental pathways and cell types for in vitro differentiated hES cells. Gene expression analysis of single cells or a small number of cells from human or nonhuman embryonic or fetal tissues provides another means to generate a database of unique gene expression profiles for distinct populations of cells at different stages of differentiation. Gene expression analysis on single cells isolated from specific tissues may be performed as previously described by Kurimoto et al., Nucleic Acids Research (2006) Vol. 34, No. 5, e42. Thus, cellular miRNA profiles on their own or in conjunction with gene expression profiles, immunocytochemistry, and proteomics provide molecular signatures that can be used to identify the tissue and developmental stage of differentiating cell lines. This technique illustrates that the database may be used to accurately identify cell types and distinguish them from other cell types.

The cells of the present invention are also useful in providing a subset of gene expression markers that are expressed at relatively high levels in some cell lines while not be expressed at all in other cell lines as opposed to genes expressed in all cell lines but at different levels of expression. This subset of “all-or none” markers can be easily identified by comparing the levels of expression as measured for instance through the use of oligonucleotide probes or other means know in the art, and comparing the level of a gene's expression in one line compared to all the other lines of the present invention. Those genes that are expressed at relatively high levels in a subset of lines, and not at all in other lines, are used to generate a short list of gene expression markers. When applied to the cells and gene expression data described herein, where negative expression in Illumina 1 is <120 RFU and positive expression is >140 RFU.

### Oil Red-O Staining

Oil Red-O staining is used to identify adipogenic differentiation. Oil Red-O was purchased from Sigma-Aldrich Cat#O1391-500ML. Cells, cells attached to cell culture vessels, or cell/matrix constructs such as HyStem beads containing cells are fixed with 4% paraformaldehyde for 30 minutes. Cells or the above-mentioned constructs are then rinsed with distilled water and stained for 10 minutes at room temperature with filtered working solution of Oil Red-O solution (3 parts 0.5% stock aqueous Oil Red-O diluted with 2 Parts H₂O), then filtered with Whatman paper. Stock solution of Oil Red-O is 0.5% (w/v) Oil Red-O in isopropanol. The cells or constructs are then rinsed with H₂O at least 4 times before photography to document the percentage of cells displaying prominent cytoplasmic red lipid droplets.

Methods for Analyzing Gene Expression in Embryonic Progenitor Cells and their Differentiated Progeny

In some embodiments of the invention, described infra, the following methods may be useful in the analysis of gene expression in embryonic progenitor cells and their differentiated progeny, e.g their in vitro differentiated progeny.

### Isolation of RNA

RNA is prepared from cell lysates using the Rneasy mini kits (Qiagen) according to the manufacturer's instructions. Briefly, cell cultures are rinsed in PBS, then lysed in a minimal volume of the RLT lysis buffer. After incubation on ice, the cell debris is removed by centrifugation and the lysate is mixed with RLT buffer, after which ethanol is added to the mixture. The combined mixture is then loaded onto the Rneasy spin column and centrifuged; the loaded column is then washed and the purified RNA is released from the column with a minimal volume of DEPC-treated water (typically 100 μl or less). The concentration of RNA in the final eluate is determined by absorbance at 260 nm.

cDNA Synthesis

cDNA synthesis is performed using the SuperScript First Strand cDNA kit (InVitrogen; Carlsbad, Calif.). Briefly, 1 μg of purified RNA is heat denatured in the presence of random hexamers. After cooling, the first strand reaction is completed using SuperSript reverse transcriptase enzyme and associated reagents from the kit. The resulting product is further purified using QIAquick PCR Purification kits (Qiagen) according to the manufacturer's instructions. Briefly, PB buffer is added to the first strand cDNA reaction products, then the mixture is loaded onto the QIAquick spin column and centrifuged. The column is washed with PE buffer and the purified cDNA is eluted from the column using 50 μl of water.

Quantitative Real-Time PCR (qRT-PCR) Analysis

Samples for testing (template) were prepared in standard Optical 96-well reaction plates (Applied Biosystems Carlsbad, Calif., PN 4306737) consisting of 30 ng of RNA equivalent of cDNA, 0.8 uM per gene-specific custom oligonucleotide primer set (Life Technologies, Carlsbad, Calif. or Eurofins Genomics, Huntsville, Ala.), ultra-pure distilled water (Life Technologies Cat. #10977015), diluted 1:1 with 12.5 ul of Power SYBR Green PCR Master Mix (Applied Biosystems Carlsbad, Calif., Cat. #4367659) incorporating AmpliTaq Gold DNA polymerase in a total reaction volume of 25 ul. Real-Time qPCR was run using Applied Biosystems 7500 Real-Time PCR System employing SDS2.0.5 software. Amplification conditions were set at 50° C. for 2 min (stage 1), 95° C. for 10 min (stage 2), 40 cycles of 95° C. for 15 sec then 60° C. for 1 min (stage 3), with a dissociation stage (stage 4) at 95° C. for 15 sec, 60° C. for 1 min, and 95° C. for 15 sec. Ct values of amplicons were normalized to the average Ct value of GAPDH.

qPCR Primers

qPCR primer pairs are synthesized for each target gene. Briefly, primer pairs for a target gene are designed to amplify only the target mRNA sequence and optimally have annealing temperatures for their target sequences that lie in the range of 65-80° C. and unique amplification products in the size range of 80-500 bp. Primer pairs are supplied at working concentrations (10 uM) to BioTrove, Inc. (Woburn, Mass.) for production of a custom qPCR Open Array plate. OpenArray plates are designed to accommodate 56-336 primer pairs and the final manufactured plate with dried down primer pairs is provided to the service provider. Purified cDNA reaction products and SYBR green master mix are loaded into individual wells of the OpenArray plate using OpenArray autolader device (BioTrove). The plate is sealed and the qPCR and loaded into the NT Imager/Cycler device (BioTrove) for amplification. Ct values for each sample are calculated using the OpenArray application software.

Primers used:
GAPDH (NM_02046.4) f. GGCCTCCAAGGAGTAAGACC
r. AGGGGTCTACATGGCAACTG (147 bp)
UCP1 (NM_021833.4) f. AGGCGTGAAGAGCAAGGGAAA
r. CCCCATCTTCACTCAGAGACTG (89 bp)
FABP4 (NM_001442.2) f. GACCTGGACTGAAGTTCGCA-
r. ACTTGCTTGCTAAATCAGGGA (94 bp)

LOC55908 (TD26, betatrophin, C19orf80) (NM_{—}018687.5) f. CTACGGGACAGCGTGCAGC r. CAGCATGATTGGTCCTCAGTTCC (257 bp)—This particular primer pair is referred to as 1422

LOC55908 (TD26, betatrophin, C19orf80) (NM_{—}018687.5) f. GCTGACAAAGGCCAGGAACAGC r. ACCTCCCCCAGCACCTCAGC (180 bp)—This particular primer pair is referred to as 1424

LOC55908 (TD26, betatrophin, C19orf80) (NM_{—}018687.5) f. GCAAGCCTGTTGGAGACTCAG r. CTGTCCCGTAGCACCTTCT (110 bp)—This particular primer pair is referred to as 1085

### Secreted Protein Isolation Protocol 1—Conditioned Medium

Cells were grown in either their normal propagation medium (West et al., 2008, Regen Med vol. 3(3) pp. 287-308) or the differentiation conditions described herein. To obtain conditioned medium on a smaller scale (typically 1-2 L or less), the cells were grown in monolayer cultures in T150, T175 or T225 flasks (Corning or BD Falcon) in a 37° C. incubator with 10% CO₂ atmosphere. For larger volume medium collections, the cells were typically grown either in 2 L roller bottles, on microcarrier suspensions (porous such as Cytodex varieties from Sigma-Aldrich, St. Louis, Mo., or non-porous such as from SoloHill Engineering, Ann Arbor, Mich.) in spinner flasks or other bioreactors, or in hollow fiber cartridge bioreactors (GE Healthcare, Piscataway, N.J.). Prior to conditioned medium collection, the cultures were rinsed twice with PBS and then incubated for 2 hours at 37° C. in the presence of serum-free medium wherein the medium is the same basal medium as described herein for the propagation or differentiation of the cells, in order to remove fetal serum proteins. The serum-free medium was then removed and replaced with fresh medium, followed by continued as described herein at 37° C. for 24-48 hours.

The culture-conditioned medium was then collected by separation from the cell-bound vessel surface or matrix (e.g., by pouring off directly or after sedimentation) and processed further for secreted protein concentration, enrichment or purification. As deemed appropriate for the collection volume, the culture medium was first centrifuged at 500 to 10,000×g to remove residual cells and cellular debris in 15 or 50 ml centrifuge tubes or 250 ml bottles. It was then passaged through successive 1 μm or 0.45 μm and 0.2 μm filter units (Corning) to remove additional debris, and then concentrated using 10,000 MW cutoff ultrafiltration in a stirred cell or Centricon centrifuge filter (Amicon-Millipore) for smaller volumes, or using a tangential flow ultrafiltration unit (Amicon-Millipore) for larger volumes. The retained protein concentrate was then dialyzed into an appropriate buffer for subsequent purification of specific proteins, and further purified using a combination of isoelectric focusing, size exclusion chromatography, ion exchange chromatography, hydrophobic or reverse phase chromatography, antibody affinity chromatography or other well-known methods appropriate for the specific proteins. During the various steps in the purification process, collection fractions were tested for the presence and quantity of the specific secreted protein by ELISA (e.g., using BMP-2 or BMP-7 ELISA kits from R&D Systems, Minneapolis, Minn.). The purified proteins were then kept in solution or lyophilized and then stored at 4 or minus 20-80° C.

### Secreted Protein Isolation Protocol 2—Urea-Mediated Protein Extraction

In the case of some secreted proteins, interactions with the cell or ECM components may reduce the simple diffusion of factors into the medium as described above in Secreted Protein Isolation Protocol 1. A simple comparison of the yield in the two protocols will suffice to determine which protocol provides the highest yield of the desired factors. In the case of Secreted Protein Isolation Protocol 2, a low concentration of urea is added to facilitate the removal of factors. In the case of the examples provided, all urea extractions were performed two days subsequent to feeding. On the second day, cell monolayers in T-150 cell culture flasks were rinsed twice with CMF-PBS and then incubated for two hours at 37° C. in the presence of serum-free medium. The rinse with CMF-PBS and the incubation in serum-free medium together aid in the removal of fetal serum proteins from the surface of the cells. The serum-free medium was then removed and 10 ml/T150 of freshly made 200 mM urea in CMF-PBS was added. The flasks were then placed on a rocker at 37° C. for 6.0 hours. The urea solution was then removed and immediately frozen at −70° C.

### Extracellular Matrix Isolation Protocol—DOC-Mediated Preparation

Extracellular matrix proteins can be extracted using the method of Hedman et al, 1979 (Isolation of the pericellular matrix of human fibroblast cultures. J. Cell Biol. 81: 83-91). Cell layers are rinsed three times with CMF-PBS buffer at ambient temperature and then washed with 30 mL of 0.5% sodium deoxycholate (DOC), 1 mM phenylmethylsulfonylfluride (PMSF, from 0.4M solution in EtOH), CMF-PBS buffer 3×10 min. on ice while on a rocking platform. The flasks were then washed in the same manner with 2 mM Tris-HCl, pH 8.0 and 1 mM PMSF 3×5 min. The protein remaining attached to the flask was then removed in 2 mL of gel loading buffer with a rubber policeman.

### Screening of Secreted or Extracellular Matrix Proteins for Biological Activity

The cell lines and their differentiated progeny of the present invention are also useful as a means of screening diverse embryonic secretomes for varied biological activities. The cell lines of the present invention cultured at 18-21 doublings of clonal expansion express a wide array of secreted soluble and extracellular matrix genes (see US Patent Application Publication 2010/0184033 entitled “METHODS TO ACCELERATE THE ISOLATION OF NOVEL CELL STRAINS FROM PLURIPOTENT STEM CELLS AND CELLS OBTAINED THEREBY” filed on Jul. 16, 2009, incorporated herein by reference). At 21 or more doublings of clonal expansion, the cells of the present invention differentially express secreted soluble and extracellular matrix genes. These proteins, proteoglycans, cytokines, and growth factors may be harvested from the embryonic progenitor cell lines or their differentiated progeny of the present invention by various techniques known in the art including those described infra. These pools of secreted and extracellular matrix proteins may be further purified or used as mixtures of factors and used in varied in vitro or in vivo assays of biological activity as is known in the art. The secreted proteins could be used as an antigen to generate antibodies such as polyclonal or monoclonal antibodies. The antibodies in turn can be used to isolate the secreted protein. As an example, differentiated progeny expressing adipokine genes such as ADIPOQ or the gene for C19orf80 (betatrophin) could be used to isolate the adipokines from the cells or to generate antibodies specific to them. The adipokines could be used for research or therapy. The antibodies could be used to purify the adipokines from the cells described infra or other cells expressing them.

### Routine Culture of the Cell Lines E3, E72, E75, E163, C4ELSR2, C4ELS5.1, and NP110SM

Cells were thawed, cultured, and routinely dissociated with 0.25% trypsin diluted 1:3 with Ca Mg free PBS to single cells and plated onto gelatin-coated tissue culture plates. The cells lines were maintained in, and all subsequent experiments with the exception of HyStem-bead experiments, were carried out at 37° C. in a humidified atmosphere of 10% CO₂ and 5% O₂. The lines E3, E72, E75, and E163 were cultured in DMEM (Cat. No. 11960-069) and fetal bovine serum (FCS) (Cat. No. SH30070-03) which were purchased from Invitrogen and Hyclone (Logan, Utah, USA), respectively. Medium and supplements were combined according to manufacturer's instructions. The routine culture medium for the propagation of the line E75 in the progenitor (undifferentiated) state was supplemented with 10% FCS, that of E72 and E3 was supplemented with 20% FCS, and that of E163 was supplemented with 5% FCS. The cell line NP110SM was routinely cultured in medium for the propagation of the line in the progenitor (undifferentiated) state in Smooth Muscle Cell Medium 2 (Cat. No. 97064) and growth supplement (Cat. No. 39267) obtained from PromoCell GmbH (Heidelberg, Germany). The lines C4ELS5.1 and C4ELSR2 were cultured on collagen IV in EpiLife LSGS medium (Catalog number M-EPI-500-CA) supplemented with the factors at final concentrations of 2% fetal bovine serum, 3 ng/ml basic fibroblast growth factor, 10 μg/ml heparin, 1.0 μg/ml hydrocortisone, and 10 ng/ml EGF as per manufacturer's conditions.

### Chondrogenic Differentiation in HyStem BMP4/BMP7

HyStem-C (BioTime, Inc. Alameda, Calif.) (BioTime, Alameda, Calif., USA) was reconstituted following the manufacturer's instructions. Briefly, the HyStem component (thiol modified hyaluronan, 10 mg) was dissolved in 1.0 ml degassed deionized water for about 20 minutes to prepare a 1% w/v solution. The Gelin-S component (thiol modified gelatin, 10 mg) was dissolved in 1 ml degassed deionized water to prepare a 1% w/v solution, and PEGDA (PEG diacrylate, 10 mg) was dissolved in 0.5 ml degassed deionized water to prepare a 2% w/v solution. HyStem (1 ml, 1% w/v) was mixed with Gelin-S (1 ml, 1% w/v) immediately before use. Pelleted cells were resuspended in recently prepared HyStem:Gelin-S (1:1 v/v) mix described above. Upon the addition of crosslinker PEGDA, the cell suspension, at a final concentration of 2.0×10⁷ cells/ml, was aliquoted at 25 μl/aliquot four to five times into each well of 6 well plates (Corning 3516) after partial gelation to form attached beads. Following complete gelation (20 minutes), chondrogenic medium was added to each well (i.e about 4 ml/each well of a 6 well plate).

### Chondrogenic Medium:

DMEM (CellGro Cat. No. 15-013-CV, or PromoCell, Heidelberg Germany C-71219), high glucose, Pyruvate, 1 mM (Gibco Cat. 11360), Penicillin:Streptomycin (100 U/ml:100 ug/ml respectively) (Gibco Cat. No. 504284), Glutamax 2 mM (Gibco Cat. No. 35050), Dexamethasone 0.1 uM (Sigma, St. Louis, Mo., Cat. No. D1756-100), L-Proline 0.35 mM (Sigma Cat. No. D49752), 2-phospho-L-Ascorbic Acid 0.17 mM (Sigma, Cat. No. 49792, Fluka), ITS Premix (BD, Franklin Lakes, N.J., sterile Cat. No. 47743-628) final concentration 6.25 ug/ml insulin, 6.25 ug/ml transferrin, 6.25 ng/ml selenious acid, serum albumin 1.25 mg/ml, 5.35 ug/ml linoleic acid and TGFb3 10 ng/ml (R&D systems, Minneapolis Minn., Cat. No. 243-B3-010).

Plates were then placed in a humidified incubator at 37° C., ambient O₂, 10% CO₂, and the cells were fed three times weekly. At the desired time point hydrogel constructs are either fixed and processed immunohistochemical analysis or lysed using RLT (Qiagen, Valencia Calif.) with 1% beta mercaptoethanol for total RNA to analyze transcript expression using qPCR and/or whole genome microarray.

Adipogenesis Protocol 1

| | |
|---|---|
| Reagents | 1. DMEM (GibcoBRL-Cat# 11965-084) |
| | 2. Calf Serum (GibcoBRL-Cat# 16170-078) |
| | 3. Fetal Bovine Serum (GibcoBRL-Cat# 10437-028) |
| | 4. Isobutylmethylxanthine (IBMX; Sigma I-7018) |
| | 5. Dexamethasone (Sigma D-4902) |
| | 6. Insulin (Bovine; Sigma I-5500) |
| | 7. MEM Sodium Pyruvate (100 mM; GibcoBRL Cat# 11360-070) |
| | 8. Pen/Strep/Glutamine (100× P/S/G; GibcoBRL Cat# 10378-016) |
| Preparation | 1. 10% Calf Serum/DMEM: 60 mL Calf Serum; 6 mL 100 mM MEM |
| of solutions | Sodium Pyruvate; 6 mL 100× P/S/G; 500 mL DMEM. |
| | 2. 10% FBS/DMEM: 60 mL Fetal Bovine Serum (Filter Sterilized); 6 |
| | mL 100 mM MEM Sodium Pyruvate; 6 mL 100× P/S/G; 500 mL |
| | DMEM. |
| | 3. IBMX Solution (make fresh): Dissolve IBMX in a solution made of |
| | 0.5 N KOH to a final concentration of 0.0115 g/mL; filter sterilize |
| | through a 0.22 mm syringe filter. |
| | 4. Insulin Stock Solution: 167 μM (1 mg/mL) in 0.02 M HCl; Filter |
| | sterilized through 0.22 mm filter; Can store at −20° C. for long term, |
| | 4° C. short term. |
| | 5. Dexamethasone Stock Solutions: Freezer Stock 10 mM of Dex in |
| | 100% ethanol (store at −20° C.); Working Stock: Dilute Freezer stock |
| | to 1 mM in PBS; Filter sterilize and store at 4° C. |
| | 6. MDI Induction Media (10 mL/10 cm plate; 5 mL/6 cm plate); To |
| | required volume of 10% FBS/DMEM add: 1:100 IBMX; 1:1000 |
| | Insulin; 1:1000 Dexamethasone working stock. |
| | 7. Insulin Media (10 mL/10 cm plate; 5 mL/6 cm plate); To required |
| | volume of 10% FBS/DMEM add: 1:100 Insulin (final concentration |
| | 10.0 ug/mL). |
| | 8. Oil red O stock solution (0.5 g/100 ml isopropanol); Just before |
| | staining: mix 60 ml of stock with 40 ml of H₂O, let it sit for 1 hr at |
| | RT; filter through whatman paper 3MM. |
| Procedures | |
| Clonal embryonic | Cells are plated in their standard growth media (West et al., 2008, |
| preadipocyte | Regenerative Medicine vol. 3(3) pp. 287-308; see Supplementary Table |
| maintenance | I) and incubated 37° C. in 10% CO₂ and preferable in 5% ambient |
| and passage | oxygen. Cells are frequently observed to prevent them from becoming |
| | too confluent (>70%), until differentiation is induced. |
| Adipocyte | 1. Grow embryonic preadipocytes to confluency in their standard |
| Differentiation | growth media (West et al., 2008, Regenerative Medicine vol. |
| Protocol | 3(3) pp. 287-308). |
| | 2. After two days of post confluency (which is counted as day 0), |
| | stimulate the cells with MDI induction media. |
| | 3. After two days of MDI an induction medium (which is called as day |
| | 2) replace the MDI induction media with Insulin Media and |
| | feed every two days. |
| Staining procedure | 1. Aspirate media, add formaldehyde slowly and let sit for 30 min. |
| | 2. Aspirate formaldehyde and add oil red O solution to cover the well, |
| | leave 1 hr at RT. |
| | 3. Remove the stain and wash with distilled water twice. |
| | Photograph. |

Adipogenesis Protocol 2
Cells are grown to confluence in their standard growth medium (West et al., 2008,
Regenerative Medicine vol. 3(3) pp. 287-308), medium is removed and replaced
by serum-free differentiation medium (DMEM/F12 containing 1 μM bovine insulin,
100 nM hydrocortisone, 10 μg of transferrin/mL, 1 nM thyronine, 1 μM rosiglitazone,
33 μM biotin, and 17 μM pantothenic acid) to induce adipocyte differentiation for
3 d. After 3 d of culture, the medium is changed to differentiation medium without
rosiglitazone for another 5 d. The mRNA from cultured cells was extracted at 0, 2, 5, 7
and 14 d of incubation for transcript analysis as described herein.

### Adipogenesis Protocol 3

HyStem-C matrix (BioTime, Alameda, Calif.) is prepared as follows. The HyStem component (10 mg of thiol-modified hyaluronan) is dissolved in 1.0 ml of degassed deionized water for approximately 20 min to prepare a 1% w/v solution. The Gelin-S® component (10 mg of thiol-modified gelatin (BioTime)) is dissolved in 1.0 ml of degassed deionized water to prepare a 1% w/v solution, and polyethylene glycol diacrylate (10 mg of PEGDA) is dissolved in 0.5 ml of degassed deionized water to prepare a 2% w/v solution. Then, HyStem (1 ml, 1% w/v) is mixed with Gelin-S (1 ml, 1% w/v) immediately before use. Pelleted cells of the present invention are resuspended in the recently prepared HyStem:Gelin-S (1:1 v/v) mix described above. Upon the addition of the PEGDA cross-linker, the cell suspension, at a final concentration of 2.0×10⁷ cells/ml is aliquoted (25 μl/aliquot) into six-well plates (Corning® 3516; VWR, PA, USA) after partial gelation. Following complete gelation (20 min), Differentiation Medium is added to each well. Differentiation medium is high glucose DMEM (CellGro Cat. No. 15-013-CV) with Pyruvate, 1 mM (Gibco Cat. 11360), Pen:Strep 100 U/ml:100 ug/ml (Gibco Cat. No. 504284), Glutamax 2 mM (Gibco Cat. No. 35050), Dexamethasone 0.1 uM (Sigma, St. Louis, Mo., Cat. No. D1756-100), L-Proline 0.35 mM (Sigma Cat. No. D49752), 2-phospho-L-Ascorbic Acid 0.17 mM (Sigma, Cat. No. 49792, Fluka), and ITS Premix (BD, Franklin Lakes, N.J., sterile Cat. No. 47743-628) with a final concentration 6.25 ug/ml insulin, 6.25 ug/ml transferrin, 6.25 ng/ml selenious acid, serum albumin 1.25 mg/ml, 5.35 ug/ml linoleic acid. The Differentiation medium is supplemented with 50 ng/ml BMP4, 1.0 μM rosiglitazone, and 2.0 nM triiodothyronine (T3). Plates were then placed in a humidified incubator at 37° C. with ambient O₂ and 10% CO₂, and the cells were fed three-times weekly. For the last 4 hours prior to use, 10 μM CL316243 was added to the culture medium. At the desired time point, hydrogel constructs were either fixed and processed for immunohistochemical analysis or lysed using RLT (Qiagen, CA, USA) with 1% beta-mercaptoethanol for total RNA to analyze transcript expression using quantitative real-time PCR (qRT-PCR) and/or whole-genome microarray, or cryopreserved for therapeutic use.

### Confluence Adipocyte Cell Differentiation Condition:

Cells were cultured in normal propagation media until reaching confluence, then shifted to different medium for 14 days with DMEM low glucose medium, 10% FBS, Penicillin/Streptomycin, GLX, ITS, Dexamethasone 1 uM, IBMX 0.5 mM, Indomethacin 60 uM. The ITS concentrations used was 6.25 ug/ml insulin, 6.25 ug/ml transferrin, 6.25 ng/ml selenious acid, serum albumin 1.25 mg/ml, 5.35 ug/ml linoleum acid. At the designated periods of time, RNA was extracted using Qiagen RNeasy kits (Qiagen, Valencia, Calif., USA cat. #74104) according to the manufacturer's instructions. The RNA yield was maximized using Qiagen's QiaShredder (Qiagen, Valencia, Calif., USA cat. #79654) to homogenize samples following the lysis of the micromasses with RLT buffer prior to RNA extraction.

### Differentiation Medium:

Differentiation medium is high glucose DMEM (CellGro Cat. No. 15-013-CV) with Pyruvate, 1 mM (Gibco Cat. 11360), Pen:Strep 100 U/ml:100 ug/ml (Gibco Cat. No. 504284), Glutamax 2 mM (Gibco Cat. No. 35050), Dexamethasone 0.1 uM (Sigma, St. Louis, Mo., Cat. No. D1756-100), L-Proline 0.35 mM (Sigma Cat. No. D49752), 2-phospho-L-Ascorbic Acid 0.17 mM (Sigma, Cat. No. 49792, Fluka), and ITS Premix (BD, Franklin Lakes, N.J., sterile Cat. No. 47743-628) with a final concentration 6.25 ug/ml insulin, 6.25 ug/ml transferrin, 6.25 ng/ml selenious acid, serum albumin 1.25 mg/ml, 5.35 ug/ml linoleic acid. In various differentiation conditions, the Differentiation medium is supplemented with various growth factors or other differentiation factors as described herein.

### Micromass Differentiation Conditions:

The present invention utilizes micromass differentiations to generate high-density cultures, thereby predisposing hEP cells to differentiation in the presence of exogenous factors. Differentiation by micromass culture was performed according to the manufacturer's (BioTime) instructions (Chondrogenesis Differentiation Kit ES-K42). Briefly, cells were cultured in the undifferentiated state, trypsinized (0.25% w/v trypsin/EDTA (Invitrogen)), diluted 1:3 with PBS (Ca and Mg free), and resuspended at a cell density of 2.0×10⁷ cells/ml in their respective growth media. Twenty-five or more micromass aliquots (200,000 cells/10 n1 aliquot) were seeded onto Corning Tissue Culture-treated polystyrene plates or dishes. The seeded micromasses were placed in a humidified incubator at 37° C. with 5% O₂ and 10% CO₂ for 90 min to 2 h for attachment. The growth medium for each respective cell line was added, aspirated the following morning and the cells were rinsed with PBS (Ca and Mg free). Then, the media were replaced with factor-containing Differentiation Medium as described herein. Cells were maintained in a humidified incubator at 37° C. with 5% O₂ and 10% CO₂ in factor-containing Differentiation Medium, which was replaced with freshly prepared medium every 2-3 days. At the designated periods of time, RNA was extracted using Qiagen RNeasy® kits (Qiagen, cat. #74104) according to the manufacturer's instructions. The RNA yield was maximized using Qiagen's QiaShredder™ (Qiagen, cat. #79654) to homogenize samples following the lysis of the micromasses with RLT buffer prior to RNA extraction.

### HyStem Bead Differentiation Condition:

HyStem-C (BioTime, Inc. Alameda, Calif.) is a matrix composed of thiol-modified gelatin and thiolated hyaluronan crosslinked in vivo or in vitro with (polyethylene glycol diacrylate (PEGDA). We observed that clonal human embryonic progenitor cell lines such as those described in the present invention, could be efficiently differentiated in high-density cultures of said crosslinked collagen and hyaluronate in the following manner. HyStem-C® (BioTime, CA, USA) is reconstituted following the manufacturer's instructions. Briefly, the HyStem component (10 mg of thiol-modified hyaluronan) is dissolved in 1.0 ml of degassed deionized water for approximately 20 min to prepare a 1% w/v solution. The Gelin-S® component (10 mg of thiol-modified gelatin (BioTime)) is dissolved in 1.0 ml of degassed deionized water to prepare a 1% w/v solution, and polyethylene glycol diacrylate (10 mg of PEGDA) is dissolved in 0.5 ml of degassed deionized water to prepare a 2% w/v solution. Then, HyStem (1 ml, 1% w/v) is mixed with Gelin-S (1 ml, 1% w/v) immediately before use. Pelleted cells are resuspended in the recently prepared HyStem:Gelin-S (1:1 v/v) mix described above. Upon the addition of the PEGDA cross-linker, the cell suspension, at a final concentration of 2.0×10⁷ cells/ml is aliquoted (25 μl/aliquot) into six-well plates (Corning® 3516; VWR, PA, USA) after partial gelation. Following complete gelation (20 min), Differentiation Medium is added to each well. Plates were then placed in a humidified incubator at 37° C. with ambient O₂ and 10% CO₂, and the cells were fed three-times weekly. At the desired time point, hydrogel constructs were either fixed and processed for immunohistochemical analysis or lysed using RLT (Qiagen, CA, USA) with 1% beta-mercaptoethanol for total RNA to analyze transcript expression using quantitative real-time PCR (qRT-PCR) and/or whole-genome microarray.

The use of HyStem beads as a means of differentiation facilitates the accumulation of large numbers of beads with large numbers of diverse hEP cell types that can be simultaneously thawed and assayed such as in high throughput robotic systems wherein the beads are exposed to diverse differentiation conditions and their differentiation assayed by gene expression microarray or other means known in the art. It also makes possible the thawing of large numbers of cryopreserved beads and the incubation of combinations of beads with diverse types of embedded cells and subsequent analysis of changes of differentiated state such as gene expression microarray or other means known in the art.

### March Adipo 2 Adipocyte Differentiation Condition:

Cells are differentiated in HyStem Differentiation Conditions in Differentiation Medium supplemented with 100 ng/ml BMP7 and 1.0 uM Rosiglitazone for 14 days where the temperature of the incubator for the last 24 hours is set at 28 deg. C.

### March Adipo 4 Adipocyte Differentiation Condition:

Cells are differentiated in HyStem Differentiation Conditions in Differentiation Medium supplemented with 100 ng/ml BMP7 and 5.0 uM Rosiglitazone for 14 days.

### March Adipo 6 Adipocyte Differentiation Condition:

Cells are differentiated in HyStem Differentiation Conditions in Differentiation Medium supplemented with 50 ng/ml BMP4 and 5.0 uM Rosiglitazone for 14 days.

### March Adipo 7 Adipocyte Differentiation Condition:

Cells are differentiated in HyStem Differentiation Conditions in Differentiation Medium supplemented with 100 ng/ml BMP7 and 1.0 uM Rosiglitazone for 14 days wherein the last 4.0 hours of incubation included the addition of the selective beta 3-adrenergic agonist CL316243.

### HyStem Osteo Cell Differentiation Condition:

Cells were cultured in normal propagation media then were differentiated in Hystem (2.0×10⁷ cells/ml) for 21 days in DMEM (high glucose), supplemented with Pen/Strep (100 U/ml Penicillin, 100 ug/ml Streptomycin), Glutamax (2 mM), Pyruvate (100 mM), Dexamethasone (0.1 uM), L-Proline (0.35 mM), 2-phospho-L-Ascorbic Acid (0.17 mM), ITS (6.25 ug/ml insulin, 6.25 ug/ml transferrin, 6.25 ng/ml selenious acid, serum albumin 1.25 mg/ml, 5.35 ug/ml linoleic acid, beta-glycerophosphate 10 mM+BMP2 50 ng/ml).

### Gene Expression Analysis

Total RNA was extracted directly from cells growing in 6-well plates or 10 cm tissue culture dishes using Qiagen RNeasy mini kits according to the manufacturer's instructions. RNA concentrations were measured using a Beckman DU530 or Nanodrop spectrophotometer and RNA quality was determined by denaturing agarose gel electrophoresis or using an Agilent 2100 bioanalyzer. Whole-genome expression analysis was carried out using Illumina Human Ref-8v3 or Human HT-12 v4 BeadArrays, and RNA levels for certain genes were confirmed by qRT-PCR. For the Illumina BeadArrays, total RNA was linearly amplified and biotin-labeled using Illumina TotalPrep kits (Life Technologies, Temecula, Calif., USA), and cRNA was quality-controlled using an Agilent 2100 Bioanalyzer. The cRNA was hybridized to Illumina BeadChips, processed, and read using a BeadStation array reader according to the manufacturer's instructions (Illumina, San Diego, Calif., USA). Values of less than 120 relative fluorescence units (RFUs) were considered as nonspecific background signal.

Comparative mRNA Expression in Undifferentiated hEP Cell Lines

A previously reported screen of 100 diverse hES-derived clonal hEP cell lines for collagen type II, alpha I (COL2A1) mRNA expression identified seven responsive lines: 4D20.8, 7PEND24, 7SMOO32, E15, MEL2, SK11, and SM30 with site-specific gene expression (Sternberg et al, Regen Med. 2013 March; 8(2):125-44. Seven diverse human embryonic stem cell-derived chondrogenic clonal embryonic progenitor cell lines display site-specific cell fates. To screen for site-specific hEP cell lines capable of adipocyte differentiation, and in particular, to identify hEP cell lines capable of brown fat cell differentiation, diverse hEP cell lines were differentiated in conditions that may induce said BAT cell differentiation such as the culture of said progenitors in the presence of HyStem beads supplemented with BMP4 as described herein and screening for the expression of the adipokine C19ORF80, or conditions such as Adipocyte Differentiation Protocol 3 expected to induce UCP1 expression in cells capable of BAT cell differentiation and mRNA was analyzed by microarray analysis using Illumina Human HT-12 v4 bead array analysis. RFU values were rank invariant normalized and the resulting values compared as described herein. RFU values of 120 RFU or less were considered background RFU values associated with non-specific hybridization for the data presented in the present invention.

### Kits and Media

In certain embodiments the invention provides a kit for differentiating progenitor cell, such as hEP cells described infra.

In one embodiment the kit comprises a media supplemented with one or more exogenously added TGF-β superfamily member. The TGF-β superfamily member may include one or more of the following: TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal. In some embodiments the media is supplemented with a plurality of exogenously added TGF-β superfamily members. In one embodiment the media is supplemented with BMP4 and BMP7. In another embodiment, the progenitor cell line is cultured in combinations of conditions wherein the cells are cultured in a hydrogel at temperatures substantially below normal body temperature, as described infra, with or without a differentiation agent such as a member of the TGF-β superfamily, retinoic acid, agonist of PPARγ, adrenergic agonist, and thyroid hormone.

One or more of the TGFβ superfamily members described in the preceding paragraph may be provided in the media at a concentration of about 1 ng/ml, 5 ng/ml, 10 ng/ml, 15 ng/ml. 20 ng/ml, 25 ng/ml, 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1,000 ng/ml. In some embodiments of the invention the TGFβ superfamily members described in the preceding paragraph may be provided in the media at a concentration of greater than 1,000 ng/ml. The TGFβ superfamily members may be chosen from TGF-beta proteins including TGFβ3, Bone Morphogenetic Proteins (BMPs) including BMP2, 4, 6, and 7, Growth Differentiation Factors (GDFs) including GDF5, Glial-derived Neurotrophic Factors (GDNFs), Activins, Lefty, Mülllerian Inhibiting Substance (MIS), Inhibins, and Nodal.

In some embodiments the kit may comprise a media supplemented with an exogenously added retinol, such as retinoic acid. The exogenously added retinoic acid may be provided at a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM. In some embodiments the concentration of the exogenously added retinoic acid is greater than 5.0 μM.

In some embodiments the kit may further comprise a hydrogel. The hydrogel may be comprised of hyaluronate, gelatin and an acrylate. The hyaluronate may be thiolated. The gelatin may be thiolated. The acrylate may be a PEG acrylate such as PEG diacrylate.

In certain embodiments of the invention the kit may further comprise a cell described infra. Thus, in some embodiments, the kit may further comprise a progenitor cell, such as a hEP cell. The hEP cell may have chondrogenic potential. In other embodiments, the kit may further comprise a differentiated progeny of a progenitor cell, such as an in vitro differentiated progeny of a progenitor cell described infra.

### Biological Deposits

Some cell lines described in this application have been deposited with the American Type Culture Collection (“ATCC”; P.O. Box 1549, Manassas, Va. 20108, USA) under the Budapest Treaty. The cell line E72 was deposited at the ATCC on May 30, 2013 and has ATCC Accession No. PTA-120380. The cell line E75 was deposited at the ATCC on May 30, 2013 and has ATCC Accession No. PTA-120381.

### Example 1

### Analysis of Effects of Differentiation Conditions on the Differentiation Cell Lines E3, E72, E75, E163, and NP110SM to Brown Fat Cells

Comparative Gene Expression when hEP Cells Differentiated into FABP4+ Adipocytes

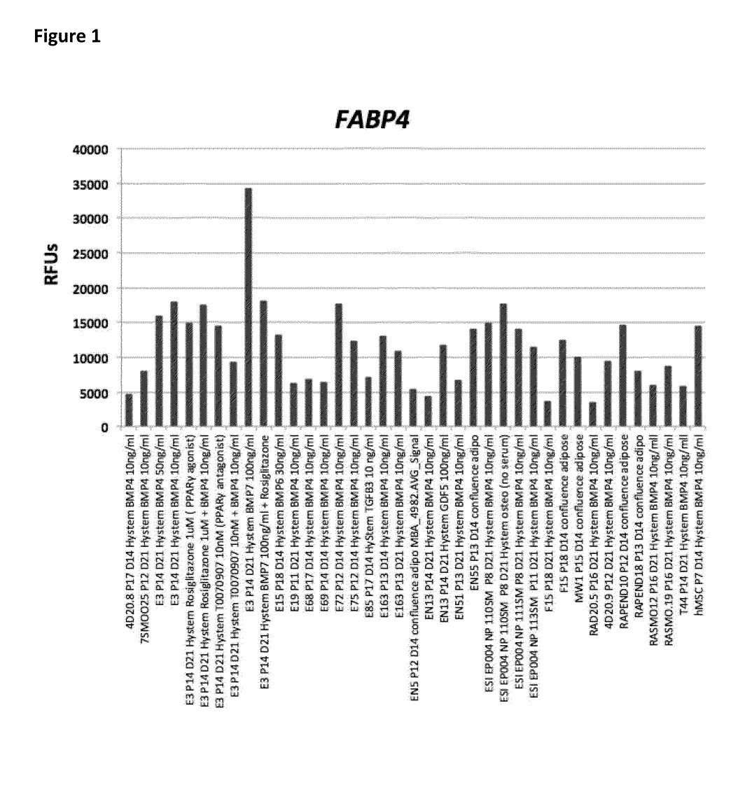

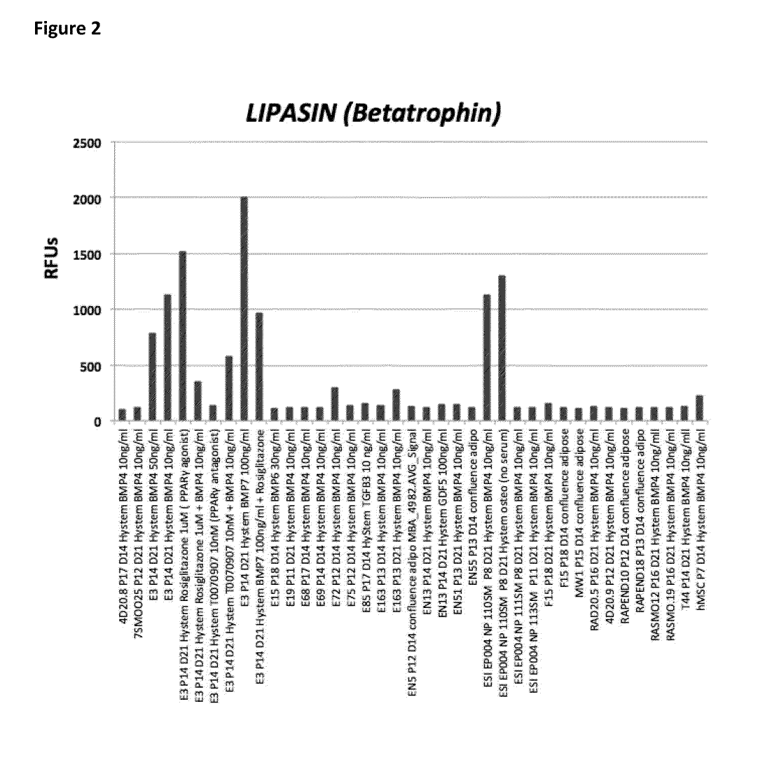

Only a subset of diverse clonal hEP cell lines studied gave rise to FABP4+ adipocytes in the presence of the “HyStem BMP4/BMP7”, “confluence adipo”, and “HyStem Osteo” conditions tested. We observed that selected lines would often respond to one of the three conditions by markedly up-regulating FABP4, but could not respond in the other conditions tested in a manner that could not be predicted prior to performing the experiment. A representative of the subset of lines with robust up-regulation of FABP4 in the three conditions are shown in FIG. 1. Of these FABP4+ differentiated clonal hEP cell lines, a subset including E3, E72, E163, and NP110SM showed an up-regulation of BETATROPHIN (also known as C19ORF80, LOC55908, and C19Orf80) (FIG. 2). Bone marrow MSCs also showed a low but detectable up-regulation of C19ORF80. As shown in FIG. 2, the cell line E3 and NP110SM showed the highest expression of C19ORF80 transcript in HyStem beads supplemented with 10 ng/ml BMP4, or in the case of NP110SM in HyStem beads supplemented with 10 ng/ml BMP4 or HyStem osteo cell differentiation conditions. When the cell line E3 was incubated in HyStem with the PPARγ agonist rosiglitazone (1.0 μM), a similar up-regulation of C19ORF80 was observed. Cells differentiated in the presence of HyStem with 10 ng/ml BMP4 but also with the PPARγ antagonist T0070907 showed decreased C19ORF80 expression compared to HyStem BMP4 alone. The line E3 when differentiated in HyStem supplemented with 100 ng/ml of BMP7 showed the highest expression of C19ORF80, while E3 cultured in 100 ng/ml of BMP7 together with 1.0 μM rosiglitazone showed high levels of C19ORF80 though lower than 100 ng/ml of BMP7 alone.

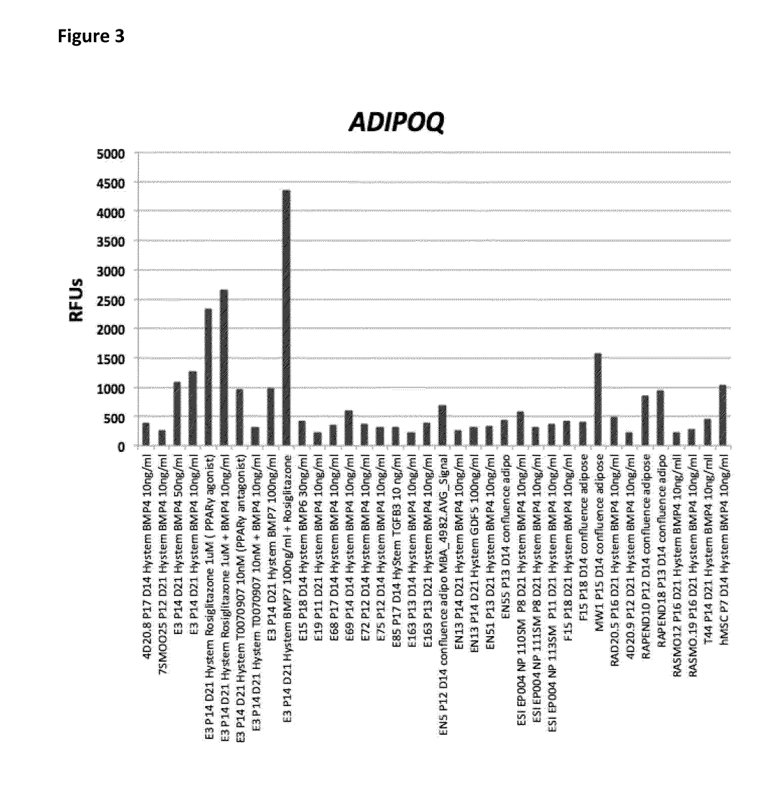

As shown in FIG. 3, transcript for the important adipokine adiponectin (ADIPOQ) was expressed at relatively high levels (>5,000 RFUs) in the cell line E3 when differentiated in HyStem beads with 1.0 μM rosiglitazone and similarly high in HyStem beads with rosiglitazone with 10 ng/ml BMP4 or 100 ng/ml of BMP7.

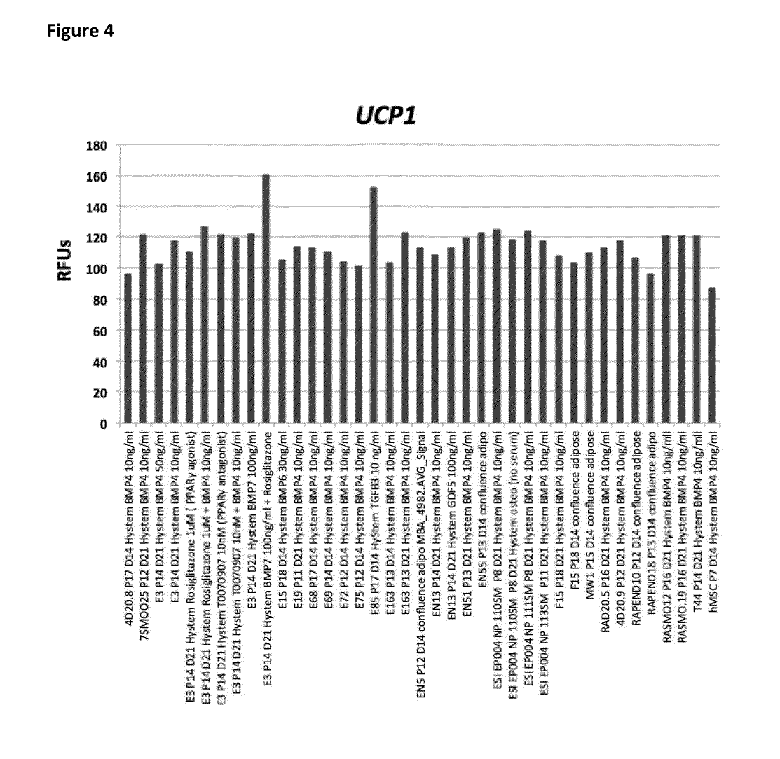

As shown in FIG. 4, transcript for the brown fat cell marker UCP1 was expressed at low but detectable levels of 161 RFUs by microarray analysis (values greater than 160 RFUs being above background) in the cell line E3 when differentiated in HyStem beads with 1.0 μM rosiglitazone in combination with 100 ng/ml of BMP7.

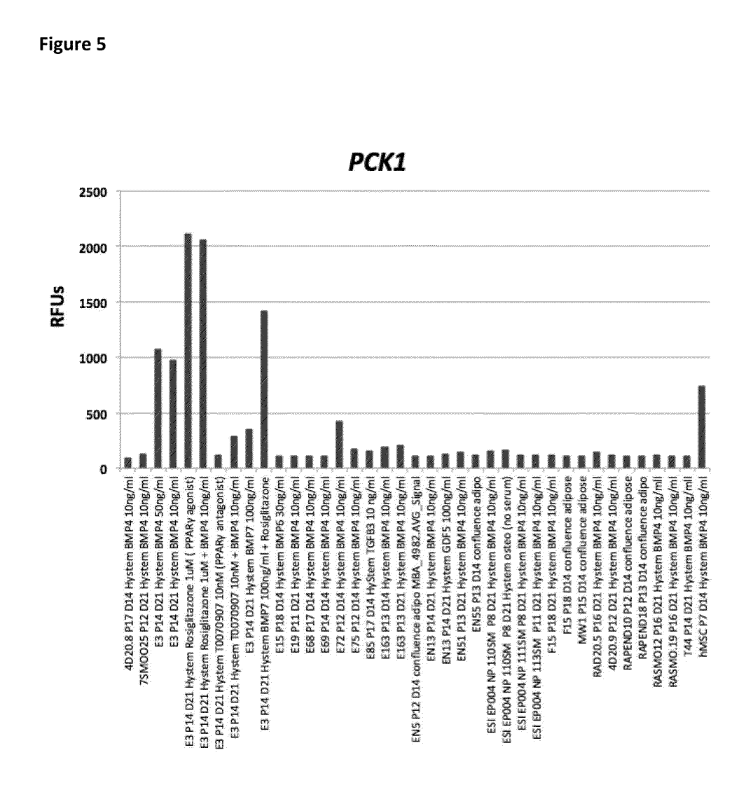

As shown in FIG. 5, the transcript PCK1 was expressed at relatively high levels in the cell line E3 when differentiated in HyStem beads with 10 ng/ml BMP4 or 100 ng/ml of BMP7 or 1.0 μM rosiglitazone in combination with 100 ng/ml of BMP7.

As shown in FIG. 6, the site-specific transcript NNAT was expressed at relatively high levels in the differentiated cell lines E3, E72, E75, E163, but not NP110SM or MSCs when differentiated in conditions that lead to FABP4 expression.

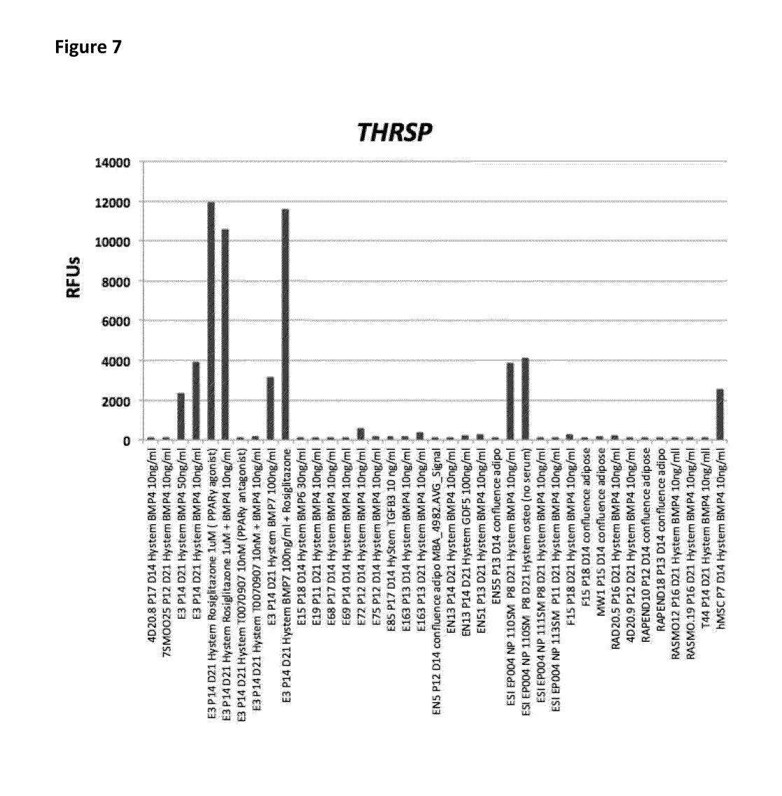

As shown in FIG. 7, the site-specific transcript THRSP was expressed at relatively high levels in the differentiated cell lines E3, E72, E75, E163, NP110SM and MSCs when differentiated in conditions that lead to FABP4 expression. THRSP expression was increased in the presence of 1.0 μM rosiglitazone and decreased to nearly undetectable levels in the presence of the PPARγ antagonist T0070907.

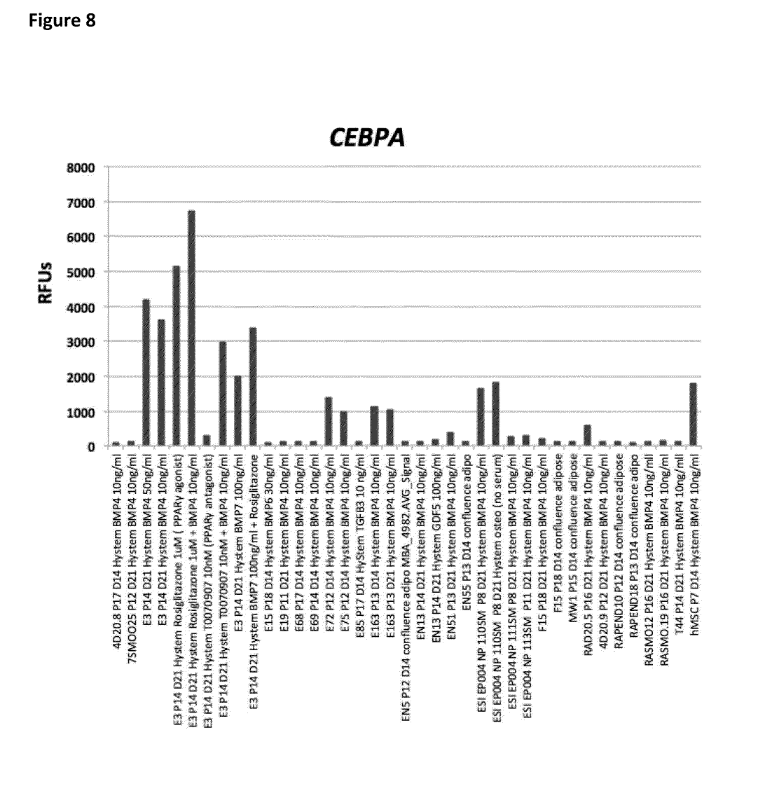

As shown in FIG. 8, the site-specific transcript CEBPA was expressed at relatively high levels specifically in the differentiated cell lines E3, E72, E75, E163, NP110SM and MSCs when differentiated in conditions that lead to FABP4 expression. CEBPA is reported to be a transcriptional activator of the BAT-specific gene UCP1 (SCIENCE SIGNALING 12 Jan. 2010 Vol 3 Issue 104 pe2).

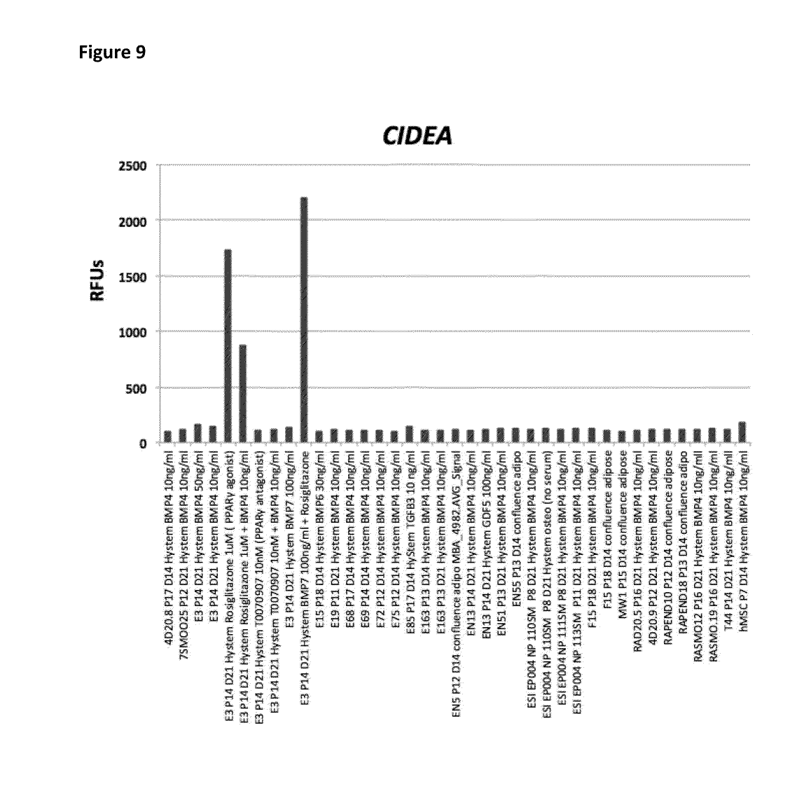

As shown in FIG. 9, the site-specific transcript CIDEA reported to be specifically expressed in BAT cells (SCIENCE SIGNALING 12 Jan. 2010 Vol 3 Issue 104 pe2) was expressed at relatively high levels in the differentiated cell line E3 in the presence of 1.0 μM rosiglitazone.

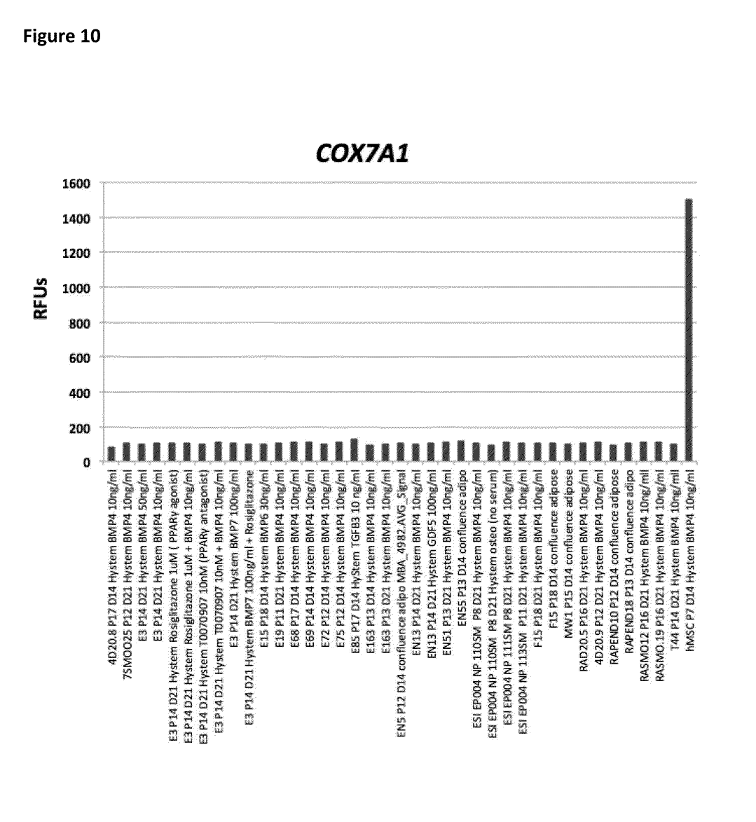

While it has been reported that COX7A1 is a specific marker of BAT cells (SCIENCE SIGNALING 12 Jan. 2010 Vol 3 Issue 104 pe2), COX7A1 was not observed in the undifferentiated or differentiated lines C4ELS5.1, E3, E72, E75, E163, NP110SM, but was expressed in the majority of adult-derived mesenchymal cells including bone marrow-derived MSCs (FIG. 10). Therefore, it can be concluded that the brown adipocyte cells of the present invention are unique and distinguishable from current brown fat cells known in the art.

### Example 2

### UCP1-Expressing Brown Fat Progenitors Derived from EYA4-Expressing Clonal Embryonic Progenitors Generated Using Adipogenesis Protocol 3

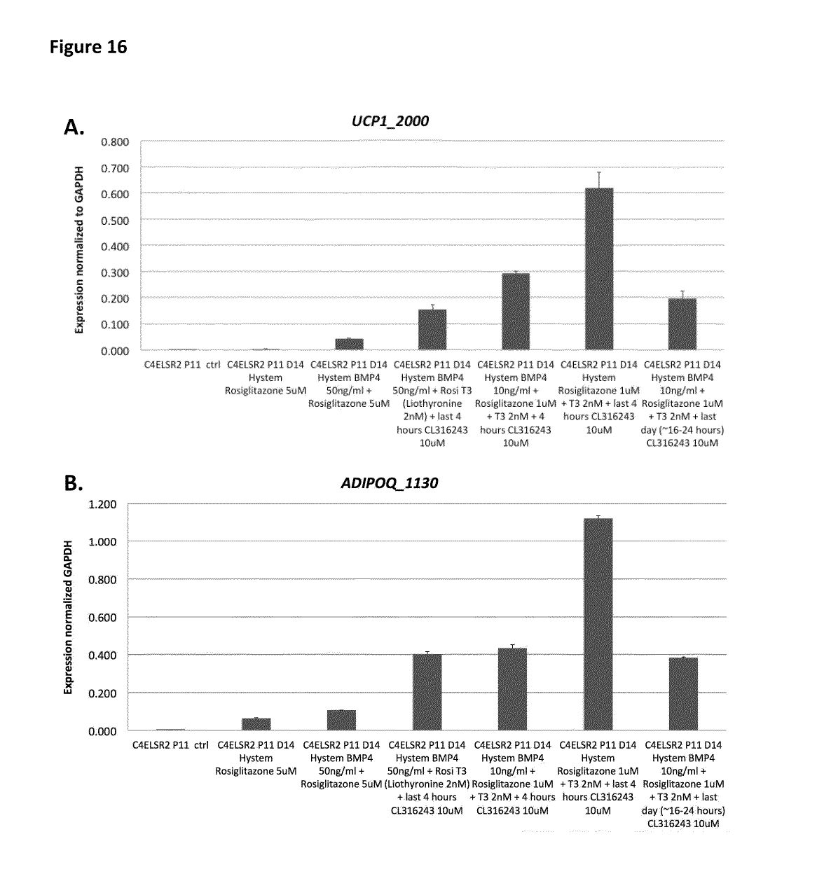

To discover improved differentiation conditions for EYA4-expressing clonal hES cell-derived progenitor lines capable of BAT cell differentiation, a novel candidate clonal embryonic cutaneous adipocyte progenitor cell (ECAPC) designated C4ELSR2 was screened in the diverse BAT cell differentiation conditions described herein. The line C4ELSR2 differs from the previously-disclosed cell type C4ELS5.1 in that the line C4ELSR2 expresses ZIC2 (accession number NM_{—}007129.2, Illumina Probe ID 510368) when analyzed for gene expression in the quiescent progenitor state (designated Crtl in FIG. 16), while the line neither C4ELS5.1 or any other of the clonal progenitor lines of the present invention such as E3, E72, E75, NP110SM, or fBAT cells express ZIC2 in either the differentiated or undifferentiated state.

The EYA4+, ZIC2+ cell line C4ELSR2 did not express adipose markers such as FABP4 in the control quiescent progenitor state, but when induced by factors disclosed in the instant application, the line induced markers of adipocytes such as FABP4 as determined by qPCR (data not shown), and markers of BAT cells such as UCP1 and ADIPOQ as also measured by qPCR (FIG. 16). Levels of UCP1 exceeded 0.1× the levels of GAPDH using the primer sets described herein, and levels of ADIPOQ exceeded 0.2× the levels of GAPDH in all conditions except those cultured in HyStem beads for 14 days in 5.0 μM rosiglitazone alone, or 50 ng/ml of BMP4 together with 5.0 μM rosiglitazone. Conditions with the highest level of efficacy are shown in FIG. 16. The highest levels of UCP1 and ADIPOQ expression were observed in differentiation conditions described as Adipogenesis Protocol 3 supra.

### Example 3

### BAT Cells Manufactured from Universal Donor cGMP Human ES Cell Lines

Clinical grade cGMP-compatible human ES cell lines are genetically modified to constitutively express CTLA4-Ig and PD-L1 (Z. Rong et al, An Effective Approach to Prevent Immune Rejection of Human ESC-Derived Allografts, Cell Stem Cell, 14: 121-130 (2014) incorporated herein by reference. In brief, the human ES cell lines described by J. Crook et al, The Generation of Six Clinical-Grade Human Embryonic Stem Cell Lines, Cell Stem Cell 1, November 2007, are genetically-modified to constitutively express the genes CTLA4-Ig and PD-L1 using a BAC-based targeting vector such as the HPRT BAC clone RP11-671P4 (Invitrogen) and the targeting vector is constructed using recombineering as described (Rong et al, A scalable approach to prevent teratoma formation of human embryonic stem cells, J. Biol. Chem. 287: 32338-32345; Song et al, Modeling disease in human ESCs using an efficient BAC-based homologous recombination system, Cell Stem Cell 6: 80-89.) incorporated herein by reference. The pCAG/CTLA4-Ig/IRES/PD-L1/poly A expression cassette is placed 600 bp downstream of the HPRT1 stop codon and the Loxp-flanked selection cassette pCAG/Neo/IRES/Puro/polyA was placed between the HPRT1 stop codon and its polyA site and Cre-mediated deletion of the selection cassette then yielded the normal expression of HPRT.

The genetic modifications are performed in cGMP conditions and the genome of the hES cells is sequenced to document the insertion site of the exogenous genes and to document the normality of the cells as described (Funk, W. D. Evaluating the genomic and sequence integrity of human ES cell lines; comparison to normal genomes Stem Cell Research (2012) 8, 154-164). Master cell banks and working cell banks are established and the working cell banks are differentiated into the BAT cellular components described herein, including betatrophin and adiponectin-expressing adipocytes as well as UCP1-expressing adipocytes, and vascular endothelial cells in combination as described herein.

### Example 4

### UCP1-Expressing Brown Fat Progenitors Derived from EYA4-Expressing Clonal Embryonic Progenitors Using March Adipo 2 Adipocyte Differentiation Condition, March Adipo 4 Adipocyte Differentiation Condition, March Adipo 6 Adipocyte Differentiation Condition, and March Adipo 7 Adipocyte Differentiation Conditions

To obtain hES-derived clonal progenitor lines of the present invention capable of differentiating into cutaneous adipocytes, designated herein as clonal embryonic cutaneous adipocyte progenitor cells (ECAPCs), and specifically, to obtain cells capable of differentiating into brown fat cells expressing UCP1, the progenitor line designated C4ELS5.1 which in the progenitor state expresses EYA4 was differentiated in parallel with the EYA4-cell line E85 in the differentiation conditions designated herein.

More specifically, the cell line of the present invention designated C4ELS5.1 at passage 14 expressing TAC1 (accession number NM_{—}013996.1, Illumina ID 6860594), EBF2 (accession number NM_{—}022659.2, Illumina ID 1030482), SCARA5 (accession number NM_{—}173833.4, Illumina ID 1030477), EYA4 (accession number NM_{—}004100.3, Illumina ID 1260180), TBX1 (accession number NM_{—}005992.1, Illumina probe ID 4880730), and FOXF2 (accession number NM_{—}001452.1, Illumina probe ID 1660470), but not expressing HOXA10 (accession number NM_{—}153715.2, Illumina ID 3290427), MKX (accession number NM_{—}173576.1, Illumina ID 6620017), or the lateral plate mesoderm marker HOXB6 (Accession number NM_{—}018952.4, Illumina ID 6220189) when propagated in the relatively undifferentiated progenitor state, and the clonal progenitor cell line E85 at passage 18 that did not express TAC1 (accession number NM_{—}013996.1, Illumina ID 6860594) or EYA4 (accession number NM_{—}004100.3, Illumina ID 1260180), but did express MKX (accession number NM_{—}173576.1, Illumina ID 6620017) when propagated in the relatively undifferentiated progenitor state, were differentiated according to March adipo 2 Adipocyte Differentiation Condition, March adipo 4 Adipocyte Differentiation Condition, March adipo 6 Adipocyte Differentiation Condition, and March adipo 7 Adipocyte Differentiation Conditions as disclosed herein for 14 days, and gene expression was analyzed as described herein to detect EYA4 positive embryonic progenitors capable of undergoing differentiation into cutaneous brown adipocytes expressing FABP4 as a non-specific adipocyte marker and UCP1 as a marker of brown adipose tissue cells capable of uncoupling oxidative phosphorylation leading to heat generation and causing weight loss when transplanted in vivo.

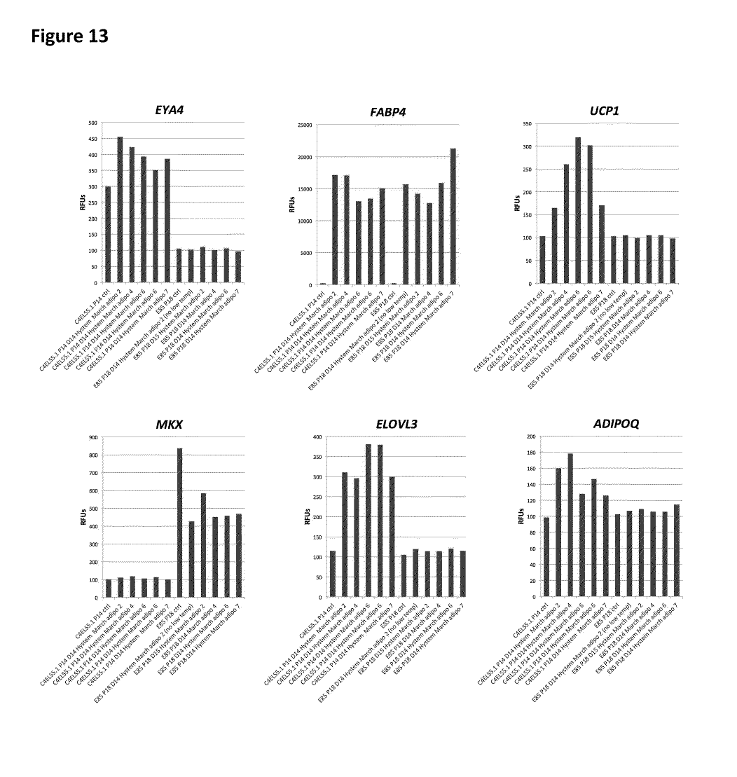

As shown in FIG. 13, the cell line C4ELS5.1 expressed EYA4 in both the control (ctrl, progenitor state), and in the differentiation conditions used in this example, but did not express MKX under ctrl or differentiated conditions, while the line E85 did not express EYA4 in ctrl or differentiated conditions, but did express MKX in all conditions. While both C4ELS5.1 and E85 robustly induced the expression of the adipocyte marker FABP4, only C4ELS5.1 expressed UCP1 (values<120 RFUs in this particular experiment being considered background signal). Only C4ELS5.1 but not E85 induced the expression of the gene ELOVL3, a gene reported to be induced by rosiglitazone in brown fat cells and to be cold-inducible. In addition, the cell line C4ELS5.1 expressed low but detectable levels of expression of ADIPOQ and C19orf80 (betatrophin) in the differentiated but not undifferentiated progenitor cells while the cell line E85 did not express these adipokines at detectable levels. Surprisingly, the genes ZIC1 and COX7A1 reported in the art to be expressed in brown fat cells, were not expressed in either C4ELS5.1 or E85 cells, suggesting that while perhaps a marker of brown fat tissue, they are not expressed in the cells of the present invention at the state of their in vitro development as taught herein.

The cell line C4ELS5.1 but not the cell line E85 was also observed to induce the expression of the following genes in the differentiation conditions used in this example: the thyroid hormone responsive gene THRSP, LMO3, BMP5, CLCA2, PTGER3, EGLN3, FLJ31568, CXCL14, ZNF423, SCNN1A, BCAN, CD1D, LIMCH1, SCGN, LOC649970, INPP5J, DACH1, KCNJ2, and PIB5PA.

C4ELS5.1 and cells with similar patterns of gene expression including TAC1 (accession number NM_{—}013996.1, Illumina ID 6860594), EBF2 (accession number NM_{—}022659.2, Illumina ID 1030482), SCARA5 (accession number NM_{—}173833.4, Illumina ID 1030477), EYA4 (accession number NM_{—}004100.3, Illumina ID 1260180), TBX1 (accession number NM_{—}005992.1, Illumina probe ID 4880730), but not expressing HOXA10 (accession number NM_{—}153715.2, Illumina ID 3290427), MKX (accession number NM_{—}173576.1, Illumina ID 6620017), or the lateral plate mesoderm marker HOXB6 (Accession number NM_{—}018952.4, Illumina ID 6220189) are therefore unknown in the art and therefore useful for the study of adipocyte differentiation, in transplantation for cosmetic surgery, for imparting weight loss, and for alleviating the symptoms of Type II diabetes, hypertension, and cardiovascular disease as described herein. Preferably, the subject is mammalian, more preferably, the subject is human. The cell of the invention can be induced to differentiate in vitro or after implantation into a patient.

In certain embodiments, the line C4ELS5.1 or cells with a similar pattern of gene expression and capable of differentiating into UCP1-expressing cells is provided to a subject in combination with a pharmaceutically acceptable carrier for a therapeutic application to an animal, including but not limited to imparting weight loss, for alleviating the symptoms of Type II diabetes, tissue repair, regeneration, reconstruction or enhancement, and the like. Said cells can, in an alternative embodiment, be administered to a host in a two- or three-dimensional matrix for a desired therapeutic purpose.

### Example 5

### Isolation of a IL13RA2+HOXA5+Clonal Human Embryonic Progenitor Cell Line from Pluripotent Stem Cells and Differentiation into BAT Cells Simultaneously Expressing Levels of UCP1, C19ORF80, and AdipoQ Comparable to Cultured fBAT Cells

The human embryonic stem cell line hES3 expressing constitutive GFP (the cell line commonly designated ENVY (Costa et al, The hESC line Envy expresses high levels of GFP in all differentiated progeny, Nat Methods 2(4):259-260 (2005))) was differentiated as described supra to generate cells with a pattern of gene expression matching NP110SM. In brief, candidate cultures expanded in NP(+) medium as described supra, were plated at clonal densities as described herein. The embryonic progenitor cell lines of the present invention designated ESI EP004NP90SM, ESI-EP004NP91SM, ESI-EP004NP92SM, EP004NP93SM, ESI-EP004NP110SM (also known as NP110SM herein) and ESI-EP004NP111SM, and ESI-EP004NP113SM were isolated colonies that formed in NP(+) medium which were subsequently harvested and plated in SM medium as described supra. These novel cell lines of the present invention at passage 11, 9, 9, 10, 8, 8, and 11 respectively were grown in 0.1% Gelatin-coated 6 well plates in the respective media. Upon confluence, media was replaced with 10% low supplement media (for each media type, dilute complete medium 1:10 with basal (no supplement) medium. The cells were allowed to become quiescent in low serum media for five days with one change of medium after three days. Cells were lysed with 350 ul/well of Qiagen RLT buffer and lysate transferred to 1.5 ml RNAes free Eppendorf tubes and stored at −80° C. until ready to send for gene expression analysis using Illumina gene expression bead arrays as described herein.

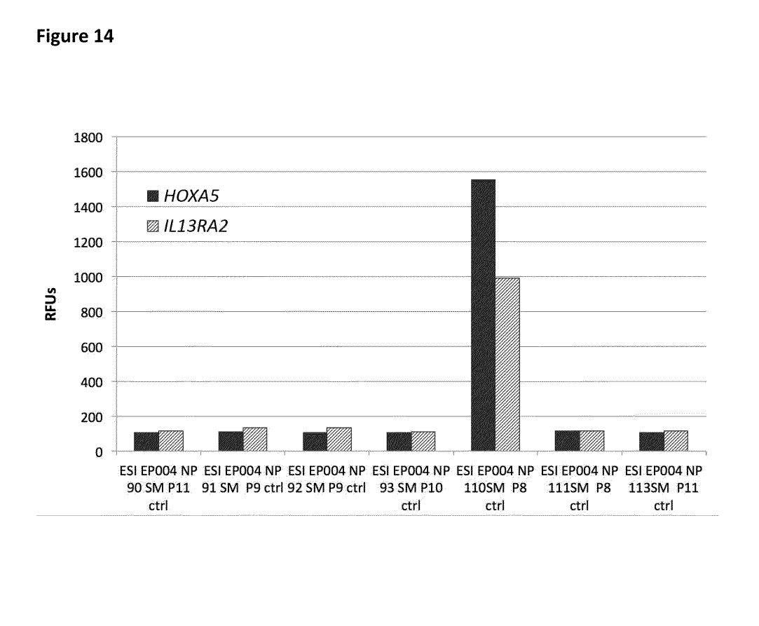

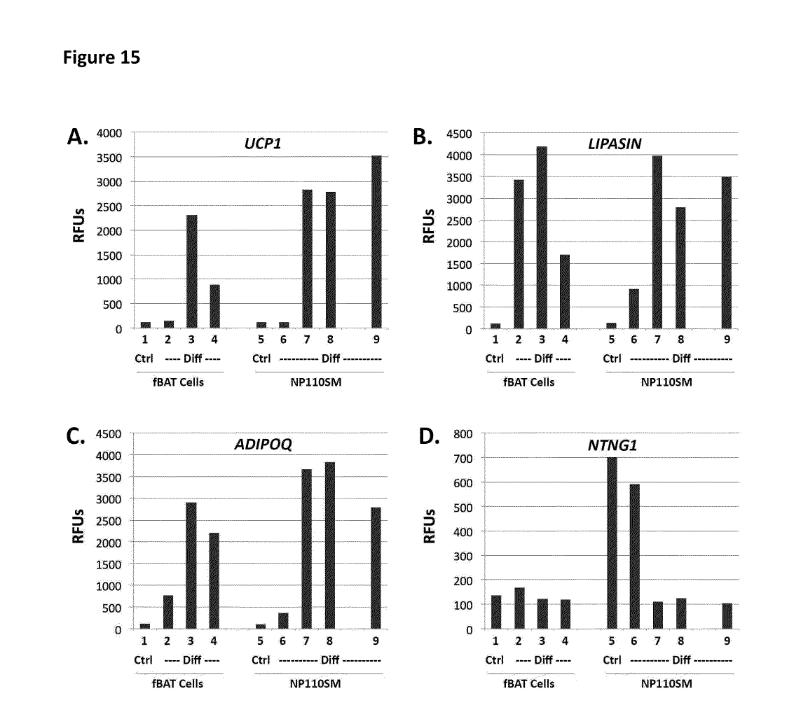

As shown in FIG. 14, the cell line NP110SM expressed both HOXA5 showed 1553 RFUs of expression and IL13RA2 showed 988 RFUs of expression while the other clones showed no or very low expression (120 RFUs and below being considered no expression). The line NP110SM was then exposed to differentiation in the BAT cell differentiation protocols described herein. Briefly, fBAT preadipocyte cells at passage 4-5 (Zenbio, Item Code CA-10, lot number 8314-102113) and NP110SM cells at passage 10 were cultured as described above in this example to induce quiescence for five days and RNA was isolated as described herein for microarray analysis. Then the fBAT and NP110SM cells were differentiated for 14 days in conditions described herein to induce differentiation of the cells into BAT cells expressing UCP1, C19ORF80, and ADIPOQ. As shown in FIG. 15, control fBAT cells and control NP110SM cells (labeled “Ctrl” and samples 1 and 5 respectively) showed low or undetectable levels of UCP1, C19ORF80, and ADIPOQ (RFU values of <120 being considered nonspecific background hybridization). Both fBAT cells and NP110SM cells induced expression of adipocyte markers such as FABP4 (accession number NM_{—}001442.1, Illumina Probe ID 150373), and CD36 (accession number NM_{—}000072.2, Illumina Probe ID 3310538) in all the differentiation conditions shown in FIG. 15 (data not shown). When differentiated for 14 days in HyStem beads supplemented in 50 ng/ml BMP4, both fBAT and NP110SM cells (FIG. 15 samples 2 and 6 respectively) responded by markedly up-regulating C19ORF80, and ADIPOQ expression, while UCP1 was expressed at a low or undetectable level in both cell types. In samples differentiated in HyStem beads for 14 days supplemented with 5.0 μM rosiglitazone, both fBAT and NP110SM cells (FIG. 15 lanes 3 and 7 respectively) markedly up-regulated UCP1, C19ORF80, and ADIPOQ at comparable levels. The combination of 50 ng/ml BMP4 and 5.0 μM rosiglitazone did not appear to further increase the expression of UCP1, C19ORF80, or ADIPOQ in fBAT or NP110SM cells (FIG. 15 lanes 4 and 8 respectively) although the addition of BMP4 appeared to improve the viability of differentiating NP110SM cells as evidenced by increased RNA yields. The addition of 10 M CL316243 together with 50 ng/ml BMP4 and 5.0 μM rosiglitazone appeared to further increase the expression of UCP1 in the NP110SM cells (FIG. 15, lane 9).

It is critical for BAT cells intended to be functionally engrafted in vivo is that the cells will recruit innervation by the sympathetic nervous system. As shown in FIG. 15, panel D, the line NP110SM expressed abundant transcript for Netrin G1, also known as Axon Guidance Molecule. Netrin G1 (NTNG1) belongs to a conserved family of proteins that act as axon guidance cues during vertebrate nervous system development (Nakashiba et al., 2000 (PubMed 10964959). NP110SM expressed the transcript for the molecule at levels much higher than cultured fBAT cells demonstrating the value of clonal embryonic stem cell lines as a means of not only generating potent BAT cells that simultaneously express levels of UCP1, C19ORF80, and ADIPOQ transcript comparable to fBAT cells, but also express levels of transcript for NTNG1 that is higher than fBAT cells, and unlike fBAT cells, the line NP110SM does not express COX7A1 when cultured as progenitors or differentiated up to 21 days in vitro, a marker of cells differentiated at least to fetal or adult stages of development, thereby showing that NP110SM cells have novel properties useful for BAT cell research and cell-based therapy for diseases associated with a loss of BAT cells such as adiposity, hypertension, Type I and Type II diabetes, lipodystrophies, and coronary disease.

What is claimed is:

1. An isolated pluripotent stem cell-derived clonal progenitor cell line capable of differentiating into an adipocyte expressing one or more markers chosen from ADIPOQ, C19orf80 or UCP1, wherein the differentiation is done in the presence of a member of TGFβ superfamily.

2. The isolated progenitor cell of claim 1, wherein the isolated progenitor cell line gives rise to brown fat cells.

3. The differentiated progeny of the isolated clonal progenitor line of claim 1 expressing C19orf80.

4. The differentiated progeny of the isolated clonal progenitor line of claim 1 expressing UCP1.

5. The differentiated progeny of the isolated clonal progenitor line of claim 1 expressing ADIPOQ.

6. A method of differentiating a clonal embryonic progenitor cell derived from human pluripotent stem cells into a cell expressing one or more of the genes UCP1, C19orf80, or ADIPOQ, comprising contacting the clonal embryonic progenitor cell with one or more of members of the TGFβ superfamily and a PPARγ agonist.

7. The method of claim 6, wherein the TGFβ superfamily member is chosen from BMP4 or BMP7.

8. The method of claim 6, wherein the PPARγ agonist is rosiglitazone.

9. The method of claim 6, wherein the progenitor cells are embedded in a mixture of hyaluronic acid and collagen-based matrix.

10. A method of obtaining an adipocyte expressing one or more markers chosen from FABP4, C19orf80, ADIPQ, UCP1, NTNG1, and THRSP, comprising contacting a pluripotent stem-cell derived clonal progenitor cell line expressing HOXA5, and IL13RA2 with one or more members of the TGFβ superfamily and a PPARγ agonist.

11. The method of claim 10 wherein the TGFβ superfamily member is a BMP.

12. The method of claim 10, wherein the BMP is BMP4.

13. The method of claim 10 wherein the PPARγ agonist is a rosiglitazone.

14. The method of claim 10 wherein the progenitor cell line is in contact with a hydrogel.

15. The method of claim 10, wherein the progenitor cell line is embedded within a hydrogel.

16. The method of claim 11, wherein the hydrogel comprises thiolated hyaluronate.

17. The method of claim 11, wherein the hydrogel comprises thiolated gelatin.

18. The method of claim 11, wherein the hydrogel comprises thiolated hyaluronate and thiolated gelatin.
